(19) United States
(12) Patent Application Publication
(10) Pub. No.: US 2023/0233701 A1
(43) Pub. Date: Jul. 27, 2023

(54) NANOBODY (VHH) CONJUGATES AND USES THERE OF

(71) Applicant: Children's Medical Center Corporation, Boston, MA (US)

(72) Inventors: Novalia Pishesha, Boston, MA (US); Hidde L. Ploegh, Boston, MA (US); Thibault Harmand, Brookline, MA (US)

(73) Assignee: Children's Medical Center Corporation, Boston, MA (US)

(21) Appl. No.: 18/007,638

(22) PCT Filed: Jun. 2, 2021
(86) PCT No.: PCT/US2021/035428

§ 371 Date: Dec. 1, 2022

Related U.S. Application Data
(60) Provisional application No. 63/154,455, filed on Feb. 26, 2021.
(60) Provisional application No. 63/033,710, filed on Jun. 2, 2020.

(51) Int. Cl. A61K 47/68; A61K 47/65; A61K 9/00; A61K 39/00

(52) U.S. Cl. CPC A61K 47/6811; A61K 47/65; A61K 47/6849; A61K 9/0019; A61K 39/0008; A61K 2039/55516; A61K 2039/6056

(57) ABSTRACT

Provided herein are compositions comprising VHH conjugates and their uses in treating diseases.

### RELATED APPLICATIONS

This Application claims the benefit under 35 U.S.C. § 119(e) of U.S. Provisional Application Ser. No. 63/033,710 entitled “NANOBODY (VHH) CONJUGATES AND USES THERE OF,” filed on Jun. 2, 2020, and of U.S. Provisional Application Ser. No. 63/154,455 entitled “NANOBODY (VHH) CONJUGATES AND USES THERE OF,” filed on Feb. 26, 2021, the entire contents of each of which are incorporated herein by reference.

### FEDERALLY SPONSORED RESEARCH

This invention was made with government support under P01DK011794 awarded by the National Institutes of Health. The government has certain rights in the invention.

### BACKGROUND

Approximately 10% of the human population suffers from an auto-immune condition, accompanied by mild to life-threatening symptoms. Current treatments for autoimmune diseases include general immunosuppression, which blunts responses across the entire spectrum of antigens. This exposes patients to an increased risk of infection and possibly even malignancies.

### SUMMARY

The present disclosure, in some aspects, provides compositions comprising one or more conjugates comprising a single domain antibody fragments (nanobodies/VHHs) conjugated to an antigen and/or an agent (e.g., an anti-inflammatory agent or a proinflammatory agent), wherein the VHH binds to a surface protein on an antigen presenting cell (APC). In some embodiments, the antigen and the agent (e.g., an anti-inflammatory agent or a proinflammatory agent) are conjugated to the same VHH. In some embodiments, the antigen and the agent (e.g., an anti-inflammatory agent or a proinflammatory agent) are conjugated to two VHHs.

The conjugates described herein engage antigen presenting cells (APCs), which under non-inflammatory conditions can lead to tolerance, whereas engagement of APCs under inflammatory conditions can elicit a strong immune response against foreign antigens. In was found surprisingly herein that, the composition of the present disclosure, when the antigen is a self-antigen and the agent is an anti-inflammatory agent, is significantly more effective in inducing immune tolerance and alleviate the symptoms of an autoimmune disease in a subject, compared to when a VHH-antigen is administered alone. Similarly, the composition of the present disclosure, when the antigen is from a pathogen and when the agent is a proinflammatory agent is significantly more effective in inducing immune response against the antigen and/or the pathogen, compared to when a VHH-antigen is administered alone.

Some aspects of the present disclosure provide compositions comprising:

(i) a conjugate comprising a single domain antibody (VHH) conjugated to an antigen and an anti-inflammatory agent, wherein the VHH binds to a surface protein on an antigen presenting cell (APC); or

(ii) a first conjugate comprising a VHH conjugated to an antigen and a second conjugate comprising a second VHH conjugated to an anti-inflammatory agent, wherein the first VHH and the second VHH bind to one or more surface proteins on an antigen presenting cell (APC). In some embodiments, the surface protein on the APC is selected from the group consisting of MHCII, CD11c, DEC205, DC-SIGN, CLEC9a, CD103, CX3CR1, CD1a, and F4/80. In some embodiments, the targeting moieties may be replaced with a natural or synthetic polypeptide, including but not limited to peptide fragments, single-chain fragment variable (scFv), diabody, Fab, or similar formats.

In some embodiments, the composition comprises a conjugate comprising a VHH to conjugated to an antigen and an anti-inflammatory agent, wherein the VHH binds to MHCII. In some embodiments, the composition comprises a first conjugate comprising a first VHH conjugated to an antigen and a second conjugate comprising a second VHH conjugated to an anti-inflammatory agent, wherein the first VHH and the second VHH both bind to MHCII. In some embodiments, the VHH comprises the amino acid sequences of SEQ ID NO: 1. In further embodiments, a VHH conjugated to an antigen or anti-inflammatory agent may have the format of a DNA or RNA molecule encoding the specified conjugate.

In some embodiments, the VHH binding to MHCII further comprises a sortase recognition sequence at the N-terminus or C-terminus. In some embodiments the sortase recognition sequence comprises the amino acid sequence LPETG (SEQ ID NO: 29). In some embodiments, the sortase recognition sequence comprises the amino acid sequence LPETGG (SEQ ID NO: 43). In some embodiments an anti-inflammatory agent or an antigen is conjugated to the VHH via the sortase recognition sequence. In some embodiments, the anti-inflammatory agent further comprises a hydrolysable or non-hydrolysable linker. In further embodiments, conjugates are produced by means of genetic fusion, other ligation enzymes (e.g., butelase, OaAEP1, subtiligase, etc.), or chemical methods (e.g., N-terminal modification using 2-pyridinecarbaldehyde (2-PCA), etc.).

In some embodiments, the composition comprises a conjugate comprising a single domain antibody (VHH) conjugated to an antigen and an anti-inflammatory agent, wherein the VHH binds to CD11c. In some embodiments, the composition comprises a first conjugate comprising a first VHH conjugated to an antigen and a second conjugate comprising a second VHH conjugated to an anti-inflammatory agent, wherein the first VHH and the second VHH both bind to CD11c. In some embodiments, the VHH comprises the amino acid sequences of SEQ ID NO: 2. In further embodiments, a VHH conjugated to an antigen or anti-inflammatory agent may have the format of a DNA or RNA molecule encoding the specified adduct.

In some embodiments, the VHH binding to CD11c further comprises a sortase recognition sequence at the N-terminus or C-terminus. In some embodiments the sortase recognition sequence comprises the amino acid sequence LPETG (SEQ ID NO: 29). In some embodiments, the sortase recognition sequence comprises the amino acid sequence LPETGG (SEQ ID NO: 43). In some embodiments an anti-inflammatory agent or an antigen is conjugated to the VHH via the sortase recognition sequence. In some embodiments, the anti-inflammatory agent further comprises a hydrolysable or non-hydrolysable linker. In further embodiments, conjugates are produced by means of genetic fusion, other ligation enzymes (e.g., butelase, OaAEP1, subtiligase, etc.), or chemical methods (e.g., N-terminal modification using 2-pyridinecarbaldehyde (2-PCA), etc.).

In some embodiments, the composition comprises a first conjugate comprising a first VHH conjugated to an antigen and a second conjugate comprising a second VHH conjugated to an anti-inflammatory agent, wherein the first VHH and the second VHH bind to different surface proteins on the APC. In some embodiments, the first VHH binds to MHCII and the second VHH binds to CD11c. In some embodiments, the first VHH binds to DEC205 and the second VHH binds to MHCII.

In some embodiments, the anti-inflammatory agent is a steroidal anti-inflammatory agent selected from the group consisting of: dexamethasone, prednisone, prednisolone, triamcinolone, methylprednisolone, and bethamethasone. In some embodiments, the anti-inflammatory agent is a nonsteroidal anti-inflammatory agent selected from the group consisting of: aspirin, celecoxib, diclofenac, ibuprofen, ketoprofen, naproxen, oxaprozin, piroxicam, cyclosporin A, and calcitriol. In some embodiments, the anti-inflammatory agent is an anti-inflammatory cytokine selected from the group consisting of IL-10, IL-35, IL-4, IL-11, IL-13, and TGFβ.

In some embodiments, the antigen comprises a polypeptide, a polysaccharide, a carbohydrate, a lipid, a nucleic acid, or combination thereof. In some embodiments, the antigen is a self-antigen. In some embodiments, the self-antigen is selected from myelin oligodendrocyte glycoprotein, myelin proteolipid protein, citrullinated fibrinogen, insulin, chromogranin A, glutamic acid decarboxylase 65-kilodalton isoform (GAD65), desmoglein 1 (DSG1), desmoglein 3 (DSG3), acetylcholine receptor (AChR), muscle-specific tyrosine kinase (MuSK), ribonucleoproteins. In some embodiments, the antigen comprises a protein used in a protein replacement therapy or a gene therapy. In some embodiments, the antigen is selected from Factor IX, Factor VIII, insulin, and AAV-derived proteins.

Other aspects of the present disclosure provide methods comprising administering to a subject in need thereof the compositions described herein. In some embodiments, the composition administered comprises (i) a conjugate comprising a single domain antibody (VHH) conjugated to an antigen and an anti-inflammatory agent, wherein the VHH binds to a surface protein on an antigen presenting cell (APC); or (ii) a first conjugate comprising a VHH conjugated to an antigen and a second conjugate comprising a second VHH conjugated to an anti-inflammatory agent, wherein the first VHH and the second VHH bind to one or more surface proteins on an antigen presenting cell (APC). In some embodiments, the method is for inducing immune tolerance to an antigen. In some embodiments, the method is for treating an autoimmune disease. In some embodiments, the autoimmune disease is selected from the group consisting of autoimmune encephalomyelitis, multiple sclerosis, type I diabetes, Pemphigus vulgaris, myasthenia gravis, lupus, celiac diseases, and inflammatory bowel disease (IBD). In some embodiments, the administration is intravenous. In some embodiments, the subject is human.

Other aspects of the present disclosure provide compositions comprising:

(i) a conjugate comprising a single domain antibody (VHH) conjugated to an antigen and a pro-inflammatory agent, wherein the VHH binds to a surface protein on an antigen presenting cell (APC); or

(ii) a first conjugate comprising a VHH conjugated to an antigen and a second conjugate comprising a second VHH conjugated to a pro-inflammatory agent, wherein the first VHH and the second VHH bind to one or more surface proteins on an antigen presenting cell (APC). In some embodiments, the surface protein on the APC is selected from the group consisting of MHCII, CD11c, DEC205, DC-SIGN, CLEC9a, CD103, CX3CR1, CD1a, and F4/80. In some embodiments, the targeting moieties may be replaced with a natural or synthetic polypeptide, including but not limited to peptide fragments, single-chain fragment variable (scFv), diabody, Fab, or similar formats.

In some embodiments, the composition comprises a conjugate comprising a single domain antibody (VHH) conjugated to an antigen and a pro-inflammatory agent, wherein the VHH binds to MHCII. In some embodiments, the composition comprises a first conjugate comprising a first VHH conjugated to an antigen and a second conjugate comprising a second VHH conjugated to a pro-inflammatory agent, wherein the first VHH and the second VHH both bind to MHCII. In some embodiments, the VHH comprises the amino acid sequences of SEQ ID NO: 1. In further embodiments, a VHH conjugated to an antigen or pro-inflammatory agent may have the format of a DNA or RNA molecule encoding the specified conjugate.

In some embodiments, the VHH binding to MHCII further comprises a sortase recognition sequence at the N-terminus or C-terminus. In some embodiments the sortase recognition sequence comprises the amino acid sequence LPETG (SEQ ID NO: 29). In some embodiments, the sortase recognition sequence comprises the amino acid sequence LPETGG (SEQ ID NO: 43). In some embodiments a pro-inflammatory agent or an antigen is conjugated to the VHH via the sortase recognition sequence. In some embodiments, the pro-inflammatory agent further comprises a hydrolysable or non-hydrolysable linker. In further embodiments, conjugates are produced by means of genetic fusion, other ligation enzymes (e.g., butelase, OaAEP1, subtiligase, etc.), or chemical methods (e.g., N-terminal modification using 2-pyridinecarbaldehyde (2-PCA), etc.).

In some embodiments, the composition comprises a conjugate comprising a single domain antibody (VHH) conjugated to an antigen and a pro-inflammatory agent, wherein the VHH binds to CD11c. In some embodiments, the composition comprises a first conjugate comprising a first VHH conjugated to an antigen and a second conjugate comprising a second VHH conjugated to a pro-inflammatory agent, wherein the first VHH and the second VHH both bind to CD11c. In some embodiments, the VHH comprises the amino acid sequences of SEQ ID NO: 2. In further embodiments, a VHH conjugated to an antigen or pro-inflammatory agent may have the format of a DNA or RNA molecule encoding the specified conjugate.

In some embodiments, the VHH binding to CD11c further comprises a sortase recognition sequence at the N-terminus or C-terminus. In some embodiments the sortase recognition sequence comprises the amino acid sequence LPETG (SEQ ID NO: 29). In some embodiments, the sortase recognition sequence comprises the amino acid sequence LPETGG (SEQ ID NO: 43). In some embodiments a pro-inflammatory agent or an antigen is conjugated to the VHH via the sortase recognition sequence. In some embodiments, the pro-inflammatory agent further comprises a hydrolysable or non-hydrolysable linker. In further embodiments, conjugates are produced by means of genetic fusion, other ligation enzymes (e.g., butelase, OaAEP1, subtiligase, etc.), or chemical methods (e.g., N-terminal modification using 2-pyridinecarbaldehyde (2-PCA), etc.).

In some embodiments, the composition comprises a first conjugate comprising a first VHH conjugated to an antigen and a second conjugate comprising a second VHH conjugated to a pro-inflammatory agent, wherein the first VHH and the second VHH bind to different surface proteins on the APC. In some embodiments, the first VHH binds to MHCII and the second VHH binds to CD11c. In some embodiments, the first VHH binds to DEC205 and the second VHH binds to MHCII.

In some embodiments, the pro-inflammatory agent is selected from the group consisting of: TLR9 agonist, LPS, HMGB1 proteins, IL2, IL12, and CD40L.

In some embodiments, the antigen comprises a polypeptide, a polysaccharide, a carbohydrate, a lipid, a nucleic acid, or combination thereof. In some embodiments, the antigen is from a microbial pathogen. In some embodiments, the microbial pathogen is a mycobacterium, bacterium, fungus, virus, parasite, or prion. In some embodiments, the antigen comprises a SARS-CoV-2 spike protein.

In some embodiments, the antigen is a tumor antigen.

In some embodiments, the composition is a vaccine composition.

Other aspects of the present disclosure provide methods comprising administering to a subject in need thereof the composition described herein. In some embodiments, the composition comprises (i) a conjugate comprising a single domain antibody (VHH) conjugated to an antigen and a pro-inflammatory agent, wherein the VHH binds to a surface protein on an antigen presenting cell (APC); or (ii) a first conjugate comprising a VHH conjugated to an antigen and a second conjugate comprising a second VHH conjugated to a pro-inflammatory agent, wherein the first VHH and the second VHH bind to one or more surface proteins on an antigen presenting cell (APC). In some embodiments, the method is for inducing immune response to an antigen. In some embodiments, the antigen is from a microbial pathogen and the method is for treating infection caused by a pathogen. In some embodiments, the method is therapeutic or prophylactic. In some embodiments, the antigen is a tumor antigen and the method is for treating cancer.

In some embodiments, the administration is intravenous. In some embodiments, the subject is human.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIGS. 1A to 1J: A single dose of VHH_{MHCII}-MOG₃₅₋₅₅ provides lasting protection against EAE. (FIG. 1A) Schematic for nanobody C-terminal sortase labeling with GGG-carrying antigenic peptides. (FIG. 1B) LC-MS of purified VHH_{MHCII} and VHH_{MHCII}-antigen adducts. (FIG. 1C-E) Mean disease scores of mice that received VHH-peptide prophylactic treatment at 3 (FIG. 1C), 2 (FIG. 1D), and 1 dose(s) (FIG. 1E) as indicated. Disease scores: 1, limp tail; 2, partial hind leg paralysis; 3, complete hind leg paralysis; 4, complete hind and partial front leg paralysis; and 5, moribund. **, p<0.01, two-way analysis of variance (ANOVA) with repeated measures. (FIG. 1F) Flow cytometry of Th1 and Th17 CD4+ lymphocytes in the spinal cord collected at the end point for mice that received 1 dose of VHH-antigen. Frequency of FoxP3+ CD4+ regulatory T cells is also indicated. Data shown as mean+/−SEM. n.s. not significant; *p<0.05, **p<0.01, ***p<0.001, unpaired t test with Holm-Sidak adjustment. Representative (FIG. 1G) H&E and (FIG. 1H) Luxol Fast Blue staining of spinal cord sections from mice having received a single dose of VHH-antigen adduct. Scale bars, 100 μm. (FIG. 1I) Mean disease scores of mice that received VHH-peptide prophylactic treatment at 60, 30, and 7 days prior to induction of EAE. (FIG. 1J) Mean clinical scores of VHH_{MHCII}-MOG₃₅₋₅₅-recipients subjected to multiple challenges with MOG/CFA/PTX and MOG/IFA/PTX. *p<0.05, **p<0.01, two-way analysis of variance (ANOVA) with repeated measures.

FIGS. 2A to 2F: Splenic CD11c+ dendritic cells are responsible for VHH_{MHCII}-MOG₃₅₋₅₅ tolerance induction by enhancing antigen presentation. (FIG. 2A) Biodistribution of VHH_{MHCII} in vivo. VHH_{MHCII}-Alexa 647 was injected intravenously into MHCII-GFP mice. 1.5 hours post-injection, spleen, whole blood and inguinal lymph nodes (iLNs) were collected and analyzed by flow cytometry. (FIG. 2B) Mean clinical scores of mice that received splenocytes and peripheral blood mononuclear cells (PBMCs) from mice treated with VHH_{MHCII}-OVA₃₂₃₋₃₃₉ or with VHH_{MHCII}-MOG₃₅₋₅₅. (FIG. 2C) Mean clinical scores of mice that received prophylactic treatment with VHH_{MHCII}-OVA₃₂₃₋₃₃₉ or VHH_{MHCII}-MOG₃₅₋₅₅ following depletion of the indicated cell subset prior to induction of EAE. (FIG. 2D) Mean disease scores of mice that received the indicated VHH-antigen. (FIG. 2E) LC-MS of purified VHH_{MHCII}-MOG₁₇₋₇₈. (FIG. 2F) Mean disease scores of mice that received VHH-peptide prophylactic treatment. ***p<0.001, two-way analysis of variance (ANOVA) with repeated measures.

FIGS. 3A to 3G: VHH_{MHCII}-MOG₃₅₋₅₅ upregulates co-inhibitory receptors on MOG₃₅₋₅₅-specific CD4 T cells. (FIG. 3A) Congenically marked CD45.1 mice received CellTrace Violet-labeled CD45.2 2D2 CD4 T cells a day prior to infusion of VHH-antigen. The number of 2D2 CD4 T cells in spleen, blood, and inguinal lymph nodes (iLNs) was determined by flow cytometry. (FIG. 3B) Violet trace dilution indicates proliferation of 2D2 T cells. (FIG. 3C) In a separate experiment, on day 3 post infusion, spleens were collected, CD45.2+ CD4+ TCRa3.2+ TCRb11+ cells were sorted according to the number of divisions they underwent and then processed for transcriptomic analyses by RNAseq. Volcano plots of RNA-seq data compare the 2D2 CD4 T cells in mice that received VHH_{MHCII}-MOG₃₅₋₅₅ after 3 divisions (div 3) with 2D2 CD4 T cells recovered from mice that received VHH_{MHCII}-OVA₃₂₃₋₃₃₉. (FIG. 3D) Heat map showing the expression of co-inhibitory receptors on 2D2 CD4+ T cells. (FIG. 3E) CellTrace Violet-dilution reflects proliferation of 2D2 T cells at day 3. VHH_{MHCII}-MOG₃₅₋₅₅ administration leads to a distinct pattern of phenotypic markers on 2D2 CD4 T cells. Representative flow images are shown. The mean fluorescence intensity (MFI) of each marker is plotted as the mean+/−SEM. *p<0.05, **p<0.01, ***p<0.001, unpaired t test with Holm-Sidak adjustment. (FIG. 3F) Mean disease scores of mice that received prophylactic treatment with VHH_{MHCII}-OVA₃₂₃₋₃₃₉ or VHH_{MHCII}-MOG₃₅₋₅₅ for the indicated genetic background; ***p<0.001, two-way analysis of variance (ANOVA) with repeated measures. (FIG. 3G) CD45.1 mice that received CD45.2 2D2 CD4 T cells were challenged and an infusion of VHH-antigen with MOG₃₅₋₅₅ emulsified in CFA on day 10. Spleens, blood, and iLNs were collected 5 days later. 2D2 T cells in mice that had received VHH_{MHCII}-MOG₃₅₋₅₅ failed to respond, unlike 2D2 T cells in mice injected with VHH_{MHCII}-OVA₃₂₃₋₃₃₉. Data are shown as mean+/−SEM; ***p<0.001, unpaired t test with Holm-Sidak adjustment.

FIGS. 4A to 4H: VHH_{MHCII}-antigen-mediated tolerance is antigen specific. (FIG. 4A) blood glucose levels in individual mice treated with VHH-antigen or saline to monitor T1D progression. Mice are considered hyperglycemic when glucose levels are >260 mg/dL. (FIG. 4B) Representative H&E staining of pancreas sections from mice that had received a single dose of VHH-antigen. Scale bars, 100 μm. (FIG. 4C) Mean paw thickness of Balb/c mice treated with VHH-antigen to assess progression of rheumatoid arthritis. (FIG. 4D) Representative Toluidine Blue staining of joint sections from mice that had received a single dose of VHH-antigen. Scale bars, 100 μm. (FIG. 4E) Mice (CD45.1+ CD8+ OTI T cells) received allotypically marked CD45.2+ CD8+ OTI T cells one day prior to injection of VHH_{MHCII}-ORF8₆₀₄₋₆₁₂ or VHH_{MHCII}-OVA₂₅₇₋₂₆₄ (OTI peptide). Mice were challenged with OTI peptide emulsified in CFA on day 10. Spleens, iLNs, and blood were collected 5 days later and analyzed by flow cytometry. (FIG. 4F) Splenocytes were cultured for 3 days in complete RPMI supplemented with OT1 peptide. Supernatant was collected to measure production of IFNγ by ELISA. Antibodies against OB1 peptide (FIG. 4G) and OVA protein (FIG. 4H) were measured by ELISA in sera collected from C57BL/6J recipients that received three consecutive injections of saline, VHH_{MHCII}-OB1, or equimolar amounts of free OVA. Data shown as mean+/−SEM. n.s. not significant; *p<0.05, **p<0.01, ***p<0.001, unpaired t test with Holm-Sidak adjustment.

FIGS. 5A to 5F: Therapeutic efficacy of VHH_{MHCII}-antigen adducts. (FIG. 5A) Mean disease score of mice treated with a single dose of VHH_{MHCII}-MOG₃₅₋₅₅ when the animals reached a disease score of 1 (limp tail). ˜40% ( 7/16) of mice succumbed (t), attributed to cytokine storm. (FIG. 5B) Structure of GGG-DEX and LC-MS of purified VHH_{MHCII}-DEX. (FIG. 5C) Serum levels of TNFα and IL-6 in EAE mice treated with VHH-antigen with or without co-administration of VHH_{MHCII}-DEX. (FIG. 5D-F) Mean and individual disease score for the mouse cohort treated with a dose of VHH-peptide+/−VHH_{MHCII}-DEX on the day the mouse reached a disease score of 1 (FIG. 5D), 2 (FIG. 5E), or 3 (FIG. 5F). ***, p<0.001, two-way analysis of variance (ANOVA) with repeated measures.

FIGS. 6A to 6C: Efficacy of anti-human MHCII VHH (VHH_{hMHCII})-antigen adducts. (FIG. 6A) LC-MS of purified VHH_{hMHCII} constructs. VHH_{hMHCII} recognizes all human HLA-DR products except for DRB3*01. (FIG. 6B) VHH_{hMHCII} efficacy in mouse EAE model. (FIG. 6C) VHH_{hMHCII}-citrullinated fibrinogen (CitFib) adduct. CitFib is a citrullinated fibrinogen peptide having fibrinogen alpha chain amino acids 79-91 with citrullinated R84.

FIGS. 7A to 7E: VHH_{MHCII}-mediated tolerance is primarily provided by CD11c+ APCs (FIG. 7A and FIG. 7B) Flow cytometry analyses of blood, spleen, and iLNs APC subsets targeted by VHH_{MHCII}-Alexa 647 adducts. (FIG. 7C) Mean clinical scores of mice that received splenocytes from mice that received VHH_{MHCII}-MOG₃₅₋₅₅ or VHH_{MHCII}-OVA₃₂₃₋₃₃₉. (FIG. 7D) Mean clinical scores of mice that received VHH_{MHCII}-MOG₃₅₋₅₅ with varieties of their immune cellular subsets are depleted (FIG. 7E) Mean clinical scores of mice that received VHH_{MHCII}-MOG₃₅₋₅₅ or other VHH-MOG₃₅₋₅₅.

FIG. 8: LC-MS of purified VHH_{MHCII} and VHH-antigen constructs. VHH_{MHCII} and VHH-antigen constructs were purified and analyzed by liquid chromatography-mass spectrometry (LC-MS) to verify purity and identity.

FIGS. 9A to 9E: Spinal cord CD4+ lymphocyte infiltration correlates with disease state. Individual clinical score of each that received VHH-peptide prophylactic treatment at 3 (FIG. 9A), 2 (FIG. 9C), and 1 dose(s) (FIG. 9E) as indicated. Clinical scores: 1, limp tail; 2, partial hind leg paralysis; 3, complete hind leg paralysis; 4, complete hind and partial front leg paralysis; and 5, moribund. Flow cytometry analyses of Th1 and Th17 infiltrating CD4+ lymphocytes in the spinal cord at the end point for mice that received 3 (FIG. 9B) and 2 (FIG. 9D) doses of VHH-antigen. Frequency of FoxP3+ CD4+ regulatory T cells are also indicated. Data shown as mean+/−SEM. n.s. not significant; *p<0.05, **p<0.01, ***p<0.001, unpaired t test with Holm-Sidak adjustment.

FIGS. 10A to 10B: Prophylactic treatment with VHH_{MHCII}-MOG₃₅₋₅₅ confers reduced CD4+ lymphocyte infiltration. (FIG. 10A) Individual clinical score of each that received VHH-peptide prophylactic treatment at −60 , −30, and −7 days prior to EAE induction. Clinical scores: 1, limp tail; 2, partial hind leg paralysis; 3, complete hind leg paralysis; 4, complete hind and partial front leg paralysis; and 5, moribund. (FIG. 10B) Flow cytometry analyses of Th1 and Th17 infiltrating CD4+ lymphocytes in the spinal cord at the end point for mice that received 1 dose of VHH-antigen at the indicated time points. Frequency of FoxP3+ CD4+ regulatory T cells are also indicated. Data shown as mean+/−SEM. n.s. not significant; *p<0.05, **p<0.01, ***p<0.001, unpaired t test with Holm-Sidak adjustment.

FIGS. 11A to 11B: Treatment with a single dose of VHH_{MHCII}-MOG₃₅₋₅₅ prevents signs of disease upon subsequent challenge. (FIG. 11A) Flow cytometry analyses of infiltrating CD4+ lymphocytes in the spinal cord at the end point for mice that received 1 dose of VHH-antigen followed by exposure to multiple EAE challenges. Data shown as mean+/−SEM. (FIG. 11B) Representative H&E and Luxol Fast Blue staining of spinal cord sections from these mice. Scale bars, 100 μm.

FIGS. 12A to 12B: In vitro characterization of VHH fluorophores. (FIG. 12A) Coomassie and fluorescent western blots of unmodified and modified VHHs carrying Alexa 647 generated by sortagging, i.e. VHH_{MHCII}-Alexa 647 and VHH_{control}-Alexa 647. (FIG. 12B) Flow cytometry analyses of splenocytes from MHCII-GFP mouse indicate positive correlation of VHH_{MHCII} binding and MHCII expression.

FIG. 13: In vivo biodistribution of VHH_{MHCII}. VHH_{MHCII}-Alexa 647 was intravenously injected into MHCII-GFP mice. 1.5 hours post injection, spleens were removed and analyzed by flow cytometry. The subpopulation of splenic GFP+ Alexa 647+ APCs was further dissected. cDCs (conventional DCs); pDCs (plasmacytoid DCs).

FIG. 14: Only intravenous administration of VHH_{MHCII}-MOG₃₅₋₅₅ provides significant protection against EAE. Determination whether the mode of delivery affects the VHH_{MHCII}-MOG₃₅₋₅₅ mediated protection in EAE. Mean clinical scores of mice that received VHH-peptide prophylactic treatment injected intravenously, intraperitoneally, or subcutaneously. Clinical scores: 1, limp tail; 2, partial hind leg paralysis; 3, complete hind leg paralysis; 4, complete hind and partial front leg paralysis; and 5, moribund. ***, p<0.01, two-way analysis of variance (ANOVA) with repeated measures.

FIGS. 15A to 15C: VHH_{MHCII}-MOG₃₅₋₅₅ treated splenocytes confers the most effective protection against EAE. (FIG. 15A) Individual clinical scores of mice that received splenocytes and peripheral blood mononuclear cells (PBMCs) from mice that have been treated with VHH_{MHCII}-OVA₃₂₃₋₃₃₉ or with VHH_{MHCII}-MOG₃₅₋₅₅. Clinical scores: 1, limp tail; 2, partial hind leg paralysis; 3, complete hind leg paralysis; 4, complete hind and partial front leg paralysis; and 5, moribund. ***p<0.001, two-way analysis of variance (ANOVA) with repeated measures. Composition of transferred spenocytes (FIG. 15B) and PBMCs (FIG. 15C) from experimental set up in (FIG. 15A).

FIGS. 16A to 16B: Depletion of selected cellular subsets indicate cell types that support VHH_{MHCII}-mediated antigen-specific tolerance. (FIG. 16A) Individual clinical scores of mice that received VHH_{MHCII}-OVA₃₂₃₋₃₃₉ or with VHH_{MHCII}-MOG₃₅₋₅₅ prophylactic treatment with the indicated cell subset depletion. To deplete CD8+ T cells, mice were injected with 400 μg intraperitoneally (i.p.) twice weekly beginning 2 weeks prior to VHH-antigen administration and throughout the EAE observation window. Macrophages was depleted by i.p. injection of 300 μg of anti-CSF1R every other day beginning 2 weeks prior to VHH-antigen administration and throughout the EAE observation window. Finally, to deplete DCs, a single dose of 100 ng DTX was administered (i.p.) into CD11c-DTR mice 2 days prior to VHH-antigen administration. (FIG. 16B) Flow cytometry confirmation of the depletions of CD8+ T cells, macrophages and DCs a day prior to VHH-antigen administration.

FIG. 17: VHH adducts that primarily recognize dendritic cells provide an intermediate level of protection against EAE. Individual clinical scores of mice that received the designated VHH-antigen. Clinical scores: 1, limp tail; 2, partial hind leg paralysis; 3, complete hind leg paralysis; 4, complete hind and partial front leg paralysis; and 5, moribund. ***p<0.001, two-way analysis of variance (ANOVA) with repeated measures.

FIG. 18: VHH_{MHCII}-MOG₃₅₋₅₅ confers protection against EAE in Batf3−/− mice Independent of dendritic cells. Mean clinical scores of wild type C57BL6/J or Batf3−/− mice (mice lacking CD8a+ DCs) that received the designated VHH-antigen. Clinical scores: 1, limp tail; 2, partial hind leg paralysis; 3, complete hind leg paralysis; 4, complete hind and partial front leg paralysis; and 5, moribund. ***p<0.001, two-way analysis of variance (ANOVA) with repeated measures.

FIG. 19: Imaging of CD4+ cells after treatment with VHH_{MHCII}-MOG₃₅₋₅₅. Non-invasive positron-emission tomography (PET)-CT imaging of adoptively transferred 2D2 CD4 T cells in Rag1−/− mice. In brief, 2D2 CD4 T cells were adoptively transferred into Rag1−/− mice and a day later VHH-antigen was administered. At day 3 and 10, 89Zr-labeled PEGylated anti-CD4 scFV was injected to track the in vivo distribution of 2D2 CD4 T cells in the whole body of the recipient mice.

FIGS. 20A to 20E: RNAseq analysis of 2D2 CD4 T cell populations after treatment with VHH_{MHCII}-MOG₃₅₋₅₅. (FIG. 20A) CellTrace Violet-labeled 2D2 CD4 T cells were adoptively transferred into congenically marked CD45.1 mice a day prior to infusion of VHH_{MHCII}-OVA₃₂₃₋₃₃₉ or VHH_{MHCII}-MOG₃₅₋₅₅. At day 3 post infusion, spleens were collected and CD45.2+ CD4+ TCRa3.2+ TCRb11+ cells were sorted and as indicated and processed for bulk transcriptomic analyses by RNAseq. (FIG. 20B) Principal-components plots of RNA-seq data shaded by FACS-sorted populations. (FIG. 20C) Heatmap showing some transcriptional features of CD4+ T cells. Gene ontology analyses of the top 500 genes that are upregulated (FIG. 20D) and downregulated (FIG. 20E) in 2D2 CD4 T cells in mice that received VHH_{MHCII}-MOG₃₅₋₅₅ after 3 division (div 3) as compared to 2D2 CD4 T derived from mice that received VHH_{MHCII}-OVA₃₂₃₋₃₃₉.

FIG. 21: Expression of phenotypic markers in 2D2 CD4 T cells after treatment with VHH_{MHCII}-MOG₃₅₋₅₅. CellTrace Violet-labeled 2D2 CD4 T cells were adoptively transferred into congenically marked CD45.1 mice a day prior to infusion of VHH_{MHCII}-MOG₃₅₋₅₅, VHH_{MHCII}-OVA₃₂₃₋₃₃₉, or equimolar MOG₃₅₋₅₅ peptides in the presence of PolyI:C/anti-CD40 as adjuvant. At day 3 post infusion, spleens were collected and analyzed by flow cytometry. CellTrace Violet-dilution indicates the proliferation of 2D2 T cells at day 3. VHH_{MHCII}-MOG₃₅₋₅₅ administration leads to a distinct pattern of phenotypic markers on 2D2 CD4 T cell. Representative flow images are shown and the mean fluorescent intensity (MFI) of each marker is plot as mean+/−SEM. *p<0.05, ***p<0.001, unpaired t test with Holm-Sidak adjustment.

FIGS. 22A to 22D: Regulatory T cells are required for protection against EAE conferred by treatment with VHH_{MHCII}-MOG₃₅₋₅₅. (FIG. 22A) Mean clinical scores of mice that received VHH-peptide prophylactic treatment with or without depletion of regulatory T cells (Tregs). Tregs were depleted in FoxP3-DTR mice by injecting 3 doses of 1 μg DTX i.p. at day −9, −8, −1 prior to therapy and weekly afterwards at 1 μg i.p. until end point. Clinical scores: 1, limp tail; 2, partial hind leg paralysis; 3, complete hind leg paralysis; 4, complete hind and partial front leg paralysis; and 5, moribund. ***, p<0.001, two-way analysis of variance (ANOVA) with repeated measures. (FIG. 22B) Flow cytometry confirmation of the depletions of FoxP3+ Tregs cells, a day prior to VHH-antigen administration. (FIG. 22C) 2D2 CD4 T cells were adoptively transferred into congenically marked CD45.1 mice a day prior to infusion of VHH-antigen. Mice were further challenged with MOG₃₅₋₅₅ emulsified in CFA at day 3. Spleens and iLNs were collected 7 days later. 2D2 T cells in mice infused with VHH_{MHCII}-MOG₃₅₋₅₅ failed to proliferate as effectively as 2D2 T cells in mice that received VHH_{MHCII}-OVA₃₂₉₋₃₃₉. Data are shown as mean+/−SEM. *p<0.05, ***p<0.001, unpaired t test with Holm-Sidak adjustment. (FIG. 22D) Among the 2D2 T cells, there was an increase of FoxP3+ cells. Data are shown as mean+/−SEM. **p<0.01, unpaired t test with Holm-Sidak adjustment.

FIGS. 23A to 23C: Treatment with VHH_{MHCII}-p31 could prevent type-1 diabetes (T1D). (FIG. 23A) Schematic for prophylactic T1D treatment at day 1 post-adoptive transfer of activated BDC2.5 splenocytes. Overall normoglycemic percentage of the data in FIG. 3C. p<0.001, log-rank test. (FIG. 23B) Flow cytometry analyses of infiltrating BDC2.5 CD4+ T cells in the designated organs 14 days after adoptive transfer of BDC2.5 splenocytes. Data shown as mean+/−SEM. n.s. not significant; *p<0.05, ***p<0.001, unpaired t test with Holm-Sidak adjustment. (FIG. 23C) Schematic for semi-therapeutic T1D treatment at day 5 post-adoptive transfer of activated BDC2.5 splenocytes. Blood glucose levels were measured to monitor T1D progression. Mice were considered diabetic when glucose levels were >250 mg/dL.

FIG. 24: The N-terminal glycine of insulin readily serves as a sortase nucleophile. Schematic indicating the N-terminal glycine residue of insulin that can act as a sortase nucleophile, with LC-MS analysis of VHH_{MHCII}-Insulin adducts produced.

FIGS. 25A to 25E: Treatment with VHH_{MHCII}-OVA₃₂₃₋₃₂₉ could reduce RA severity. (FIG. 25A) Individual paw thickness of the mice treated with VHH-antigens to assess RA progression. (FIG. 25B) Representative images of mouse paws at day 3 post heat-aggregated ovalbumin (HAO) challenge. (FIG. 25C) Th1 responses of popliteal lymph nodes-derived splenocytes harvested at end point (day 7 post HAO challenges). Data shown as mean+/−SEM. *p<0.05, unpaired t test with Holm-Sidak adjustment. Anti-Ovalbumin (FIG. 25D) and anti-OVA323-339 (FIG. 25E) antibody responses from the mice described in (FIG. 25A). Data shown as mean+/−SEM. *p<0.05, unpaired t test with Holm-Sidak adjustment.

FIG. 26: Mice administered VHH_{MHCII}-MOG₃₅₋₅₅ concurrent with initial symptoms of EAE display heterogeneous outcomes. Individual clinical score of mice that were treated with a dose of received VHH_{MHCII}-MOG₃₅₋₅₅ on the day the mouse reached clinical score of 1. Clinical scores: 1, limp tail; 2, partial hind leg paralysis; 3, complete hind leg paralysis; 4, complete hind and partial front leg paralysis; and 5, moribund. ˜40% ( 7/16) of mice were found dead (t) attributed to cytokine storm.

FIG. 27: Synthesis of GGG-carrying dexamethasone (DEX). Schematic indicates the steps by which VHH_{MHCII}-dexamethasone adducts are produced.

FIG. 28: Co-treatment with VHH_{MHCII}-DEX reduces spinal cord infiltration of CD4+ T cells. Clinical scores: 1, limp tail; 2, partial hind leg paralysis; 3, complete hind leg paralysis; 4, complete hind and partial front leg paralysis; and 5, moribund. Flow cytometry analyses of Th1 and Th17 infiltrating CD4+ lymphocytes in the spinal cord at the end point for each mouse. Frequency of FoxP3+ CD4+ regulatory T cells are also indicated. Data shown as mean+/−SEM. *p<0.05, **p<0.01, ***p<0.001, unpaired t test with Holm-Sidak adjustment.

FIGS. 29A to 29B: Co-treatment with free dexamethasone requires a substantially higher dose than VHH_{MHCII}-DEX. Individual clinical score of mice that were treated with a dose of received VHH_{MHCII}-MOG₃₅₋₅₅ in the presence 0.5 μg DEX (FIG. 29A) or 100 μg DEX (FIG. 29B) on the day the mouse reached clinical score of 1. Clinical scores: 1, limp tail; 2, partial hind leg paralysis; 3, complete hind leg paralysis; 4, complete hind and partial front leg paralysis; and 5, moribund. 50% ( 2/4) of mice were found dead (t) when they received only 0.5 μg DEX attributed to cytokine storm.

FIGS. 30A to 30C: Schematics of conjugation process via sortagging. Schematics indicate steps for maleimide and copper free click chemistry sortagging approaches.

FIGS. 31A to 31F: VHH_{MHCII}-Spike_{RBD} Immunization induces high titer, durable anti-Spike RBD antibodies that neutralize pseudo-typed VSV SARS-CoV-2. (FIG. 31A) Design of VHH_{MHCII}-Spike_{RBD}. (FIG. 31B) Coomassie gel of VHH_{MHCII}, the Spike_{RBD} and the VHH_{MHCII}-Spike_{RBD} fusion product produced. (FIG. 31C) Immunization scheme: C57BL/6J mice were vaccinated intraperitoneally and bled for sera as indicated: pre-immune serum is collected 3 days prior to immunization. Blood is collected at day 32 and day 150 since the first dose of immunization. (FIG. 31D) Humoral responses in sera of immunized mice were evaluated (n=4/group) by ELISA for anti-Spike RBD IgG. (FIG. 31E) IgM, IgA, IgG1, IgG2b. (FIG. 31F) Neutralization data for VSV, pseudotyped with the SARS-CoV-2 Spike glycoprotein.

FIGS. 32A to 32E: Immunization of mice with a single dose of VHH_{MHCII}-Spike_{RBD} fusion quickly elicits a strong T cell response against the Spike_{RBD}. (FIG. 32A) Immunization scheme: C57BL/6J mice were vaccinated intraperitoneally and spleen was harvested for T cell assays. (FIG. 32B) Schematic of Spike_{RBD} amino acid sequence and peptides generated for ELISPOT analyses (FIG. 32C) ELISPOT analyses of Spike_{RBD}-specific T cells in mice vaccinated with adjuvant only, Spike_{RBD}+adjuvant or VHH_{MHCII}-Spike_{RBD}+adjuvant with Spike_{RBD} was truncated into 15-mer peptides, 10 amino acid overlap and indicated as peptide 1-53. (FIG. 32D) Cytokine secretion of splenocytes at day 3 after cultured with the indicated peptides. (FIG. 32E) Flow cytometry analyses of splenocytes after incubation with or without peptide mixture (peptide 42+47+48+49) for 6 hours.

FIGS. 33A to 33D: Two doses of the VHH_{MHCII}-Spike_{RBD} vaccination suffice to generate persistent and neutralizing antibody titers against multiple variants of SARS-CoV-2. (FIG. 33A) Kinetics of the humoral responses in sera of immunized mice were evaluated (n=4/group) by ELISA for anti-Spike RBD IgG. (FIG. 33B) IgM, IgA, IgG1, IgG2b. (FIG. 33C) Antibody titer of immunized mice against Spike RBD protein with K417T, E484K, N501Y mutations. (FIG. 33D) Neutralization data for VSV, pseudotyped with the SARS-CoV-2 Spike glycoprotein Wuhan+D418G as well as the other variants.

FIGS. 34A to 34D: The VHH_{MHCII}-Spike_{RBD} adduct elicits a strong antibody response regardless of mode of delivery, storage conditions, lyophilization, and suboptimal immunity in aging mouse. (FIG. 34A) Immunization timeline. (FIG. 34B) Anti-Spike RBD IgG, IgM, IgA, IgG1, IgG2b in the sera of mice immunized using different mode of delivery. (FIG. 34C) Anti-Spike RBD IgG, IgM, IgA, IgG1, IgG2b in the sera of mice immunized using different formulation storage conditions. (FIG. 34D) Efficacy of antibody production in aged mice. Antibody titers were evaluated by ELISA (n=4/group).

### DETAILED DESCRIPTION OF CERTAIN EMBODIMENTS

The present disclosure, in some aspects, provides compositions comprising one or more conjugates (also referred to as “adducts” in the examples and figures) comprising a single domain antibody fragment (nanobodies/VHHs) conjugated to an antigen (e.g., an antigen to which immune tolerance is needed, such as a self-antigen or an exogenous enzyme used for therapy) and/or an anti-inflammatory agent, wherein the VHH binds to a surface protein on an antigen presenting cell (APC), methods of using such compositions for inducing immune tolerance to the antigen, and methods of using such compositions for treating autoimmune diseases. In some embodiments, the composition comprises a conjugate comprising a VHH conjugated to an antigen (e.g., an antigen to which immune tolerance is needed) and an anti-inflammatory agent, wherein the VHH binds to a surface protein on an antigen presenting cell (APC). In some embodiments, the composition comprises a first conjugate comprising a first VHH conjugated to an antigen (e.g., an antigen to which immune tolerance is needed) and a second conjugate comprising a second VHH conjugated to an anti-inflammatory agent, wherein the first VHH and the second VHH bind to one or more surface proteins on an antigen presenting cell (APC).

Other aspects of the present disclosure provide compositions comprising one or more conjugates comprising a VHH conjugated to an antigen (e.g., an antigen to which immune response is needed, such as an antigen from a pathogen or a tumor antigen) and/or a proinflammatory agent, wherein the VHH binds to a surface protein on an APC, methods of using such compositions to induce immune response to the antigen, and methods of using such compositions to treat infection (e.g., by a pathogen) and cancer. In some embodiments, the composition comprises a conjugate comprising a VHH conjugated to an antigen (e.g., an antigen to which immune response is needed, such as an antigen from a pathogen or a tumor antigen) and an proinflammatory agent, wherein the VHH binds to a surface protein on an antigen presenting cell (APC). In some embodiments, the composition comprises a first conjugate comprising a first VHH conjugated to an antigen (e.g., an antigen to which immune response is needed, such as an antigen from a pathogen or a tumor antigen) and a second conjugate comprising a second VHH conjugated to an proinflammatory agent, wherein the first VHH and the second VHH bind to one or more surface proteins on an antigen presenting cell (APC).

### VHHs and Conjugates

The conjugates of the present disclosure comprise a single domain antibody (also referred to as nanobody or VHH). A “single domain antibody fragment,” a “nanobody,” or a “VHH,” as used herein, refers to an antibody fragment consisting of a single monomeric variable antibody domain. It is known that Camelids produce heavy chain-only antibodies (e.g., as described in Hamers-Casterman et al., 1992, incorporated herein by reference). The single-domain variable fragments of these heavy chain-only antibodies are termed VHHs or nanobodies. VHHs retain the immunoglobulin fold shared by antibodies, using three hypervariable loops, CDR1, CDR2 and CDR3, to bind to their targets. Many VHHs bind to their targets with affinities similar to conventional full-size antibodies, but possess other properties superior to them. Therefore, VHHs are attractive tools for use in biological research and therapeutics. VHHs are usually between 10 to 15 kDa in size, and can be recombinantly expressed in high yields, both in the cytosol and in the periplasm in E. coli. VHHs can bind to their targets in mammalian cytosol. A VHH fragment (e.g., NANOBODY®) is a recombinant, antigen-specific, single-domain, variable fragment derived from camelid heavy chain antibodies. Although they are small, VHH fragments retain the full antigen-binding capacity of the full antibody. VHHs are small in size, highly soluble and stable, and have greater set of accessible epitopes, compared to traditional antibodies. They are also easy to use as the extracellular target-binding moiety of the chimeric receptor described herein, because no reformatting is required.

In some embodiments, the VHH used in the conjugates described herein binds to a surface protein on an antigen presenting cell (APC). An “antigen presenting cell (APC)” refers to a cell that displays antigen complexed with major histocompatibility complexes (MHCs) on their surfaces, a process known as antigen presentation. T cells may recognize these complexes using their T cell receptors (TCRs). Almost all cell types can present antigens in some way. They are found in a variety of tissue types. As used herein, the term “antigen presenting cells” refers to professional antigen-presenting cells including, without limitation, macrophages, B cells, and dendritic cells. Antigen-presenting cells play important roles in effective adaptive immune response, as the functioning of both cytotoxic and helper T cells is dependent on APCs. Antigen presentation allows for specificity of adaptive immunity and can contribute to immune responses against both intracellular and extracellular pathogens. It is also involved in defense against tumors. Some cancer therapies involve the creation of artificial APCs to prime the adaptive immune system to target malignant cells. Additionally, APCs also play a role in immune tolerance by presenting self-antigens to T cells, e.g., as described in Best et al., Front Immunol. 2015; 6: 360, incorporated herein by reference.

In addition to the MHC family of proteins, other specialized signaling molecules on the surfaces of both APCs and T cells are also required for antigen presentation. In some embodiments, the conjugates described herein comprise a VHH that binds to a protein on the surface of an APC, thus engaging the APC. Non-limiting examples of surface proteins on APCs that can be targeted by the VHH in the conjugates described herein include, without limitation: Major histocompatibility complex II (MHCII), Integrin, alpha X (CD11c), Lymphocyte antigen 75 (DEC205, also referred to as CD205), Dendritic Cell-Specific ICAM-3-Grabbing Non-Integrin 1 (DC-SIGN), C-Type Lectin Domain Containing 9A (CLEC9a), Integrin, alpha E (CD103), C-X3-C Motif Chemokine Receptor 1 (CX3CR1), Cluster of Differentiation 1a (CD1a), and EGF-like module-containing mucin-like hormone receptor-like 1 (F4/80, also referred to as EMR1).

In some embodiments, the VHH in the conjugates described herein binds to one surface protein on an APC (e.g., without limitation, MHCII, CD11c, DEC205, DC-SIGN, CLEC9a, CD103, CX3CR1, CD1a, or F4/80). In some embodiments, the VHH in the conjugates described herein is bi-specific or multispecific. In some embodiments, the VHH in the conjugates described herein binds one or more (e.g., 1, 2, 3, 4, 5, 6, 7, 8, 9, 10, or more) surface proteins in an APC. Any known VHHs that bind to surface proteins on APCs can be used in accordance with the present disclosure.

In some embodiments, the VHH binds to MHCII. VHHs that bind to MHCII have been described, e.g., in U.S. Pat. No. 9,751,945, incorporated herein by reference. The amino acid sequence of an example of a VHH that binds to MHCII is provided in Table 1.

In some embodiments, the VHH in the conjugates described herein comprises an amino acid sequence that is at least 80% (e.g., at least 80%, at least 85%, at least 90%, at least 95%, at least 99%) identical to the amino acid sequence of SEQ ID NO: 1. In some embodiments, the VHH in the conjugates described herein comprises an amino acid sequence that is 80%, 85%, 905, 95%, or 99% identical to the amino acid sequence of SEQ ID NO: 1. In some embodiments, the VHH in the conjugates described herein comprises the amino acid sequence of SEQ ID NO: 1.

In some embodiments, the VHH binds to CD11c. VHHs that bind to CD11c have been described, e.g., in Bannas et al., Front Immunol. 2017; 8: 1603, incorporated herein by reference. The amino acid sequence of an example of a VHH that binds to CD11c is provided in Table 1.

In some embodiments, the VHH in the conjugates described herein comprises an amino acid sequence that is at least 80% (e.g., at least 80%, at least 85%, at least 90%, at least 95%, at least 99%) identical to the amino acid sequence of SEQ ID NO: 2. In some embodiments, the VHH in the conjugates described herein comprises an amino acid sequence that is 80%, 85%, 90%, 95%, or 99% identical to the amino acid sequence of SEQ ID NO: 2. In some embodiments, the VHH in the conjugates described herein comprises the amino acid sequence of SEQ ID NO: 2.

TABLE 1
Examples of VHHs binding to surface proteins on
APC

| Target | Amino Acid Sequence |
|---|---|
| MHCII | QVQLQESGGGLVQAGDSLRLSCAASGRTFSRGVMGWFRRAP |
| | GKEREFVAIFSGSSWSGRSTYYSDSVKGRFTISRDNAKNTV |
| | YLQMNGLKPEDTAVYYCAAGYPEAYSAYGRESTYDYWGQGT |
| | QVTVSS (SEQ ID NO: 1) |
| CD11c | EVQLVESGGGLVQAGGSLRLSCRVSGLPFSNLILGWLRQAP |
| | GKEREFVARISRSDSTYYEQFAEGRFTISRDNAKNTAYLHL |
| | NDLKPEDTAVYYCAAANPIFRSYDDYDYWGQGTQVTVSS |
| | (SEQ ID NO: 2) |

In some embodiments, any one of the VHHs in the conjugates described herein comprises additional sequences such as a sortase recognition sequence (e.g., as described in U.S. Pat. No. 9,751,945, incorporated herein by reference). Enzymes identified as “sortases” from Gram-positive bacteria cleave and translocate proteins to proteoglycan moieties in intact cell walls. Among the sortases that have been isolated from Staphylococcus aureus, are sortase A (SrtA) and sortase B (SrtB).

In some embodiments, a recognition sequence of a sortase further comprises one or more additional amino acids, e.g., at the N or C terminus. For example, one or more amino acids (e.g., up to 5 amino acids) having the identity of amino acids found immediately N-terminal to, or C-terminal to, a 5 amino acid recognition sequence in a naturally occurring sortase substrate may be incorporated. Such additional amino acids may provide context that improves the recognition of the recognition motif.

Sortases have been classified into 4 classes, designated A, B, C, and D, based on sequence alignment and phylogenetic analysis of 61 sortases from Gram positive bacterial genomes (Dramsi S, Trieu-Cuot P, Bierne H, Sorting sortases: a nomenclature proposal for the various sortases of Gram-positive bacteria. Res Microbiol. 156(3):289-97, 2005. These classes correspond to the following subfamilies, into which sortases have also been classified by Comfort and Clubb (Comfort D, Clubb R T. A comparative genome analysis identifies distinct sorting pathways in gram-positive bacteria. Infect Immun., 72(5):2710-22, 2004): Class A (Subfamily 1), Class B (Subfamily 2), Class C (Subfamily 3), Class D (Subfamilies 4 and 5). The aforementioned references disclose numerous sortases and recognition motifs. See also Pallen, M. J.; Lam, A. C.; Antonio, M.; Dunbar, K. TRENDS in Microbiology, 2001, 9(3), 97-101. Those skilled in the art will readily be able to assign a sortase to the correct class based on its sequence and/or other characteristics such as those described in Drami, et al., supra. The term “sortase A” is used herein to refer to a class A sortase, usually named SrtA in any particular bacterial species, e.g., SrtA from S. aureus. Likewise, “sortase B” is used herein to refer to a class B sortase, usually named SrtB in any particular bacterial species, e.g., SrtB from S. aureus.

In some embodiments, the sortase used for producing the conjugates described herein is a sortase A (SrtA). SrtA recognizes the motif LPXTG (SEQ ID NO: 25), with common recognition motifs being, e.g., LPKTG (SEQ ID NO: 26), LPATG (SEQ ID NO: 27), LPNTG (SEQ ID NO: 28). In some embodiments LPETG (SEQ ID NO: 29) is used. However, motifs falling outside this consensus may also be recognized. For example, in some embodiments the motif comprises an ‘A’ rather than a ‘T’ at position 4, e.g., LPXAG (SEQ ID NO: 30), e.g., LPNAG (SEQ ID NO: 31). In some embodiments the motif comprises an ‘A’ rather than a ‘G’ at position 5, e.g., LPXTA (SEQ ID NO: 32), e.g., LPNTA (SEQ ID NO: 33). In some embodiments the motif comprises a ‘G’ rather than ‘P’ at position 2, e.g., LGXTG (SEQ ID NO: 34), e.g., LGATG (SEQ ID NO: 35). In some embodiments the motif comprises an ‘I’ rather than ‘L’ at position 1, e.g., IPXTG (SEQ ID NO: 36), e.g., IPNTG (SEQ ID NO: 37) or IPETG (SEQ ID NO: 38).

In some embodiments, the sortase used for producing the conjugates described herein is sortase B (SrtB), e.g., a sortase B of S. aureus, B. anthracis, or L. monocytogenes. Motifs recognized by sortases of the B class (SrtB) often fall within the consensus sequences NPXTX (SEQ ID NO: 39), e.g., NP[Q/K]-[T/s]-[N/G/s], such as NPQTN (SEQ ID NO: 40) or NPKTG (SEQ ID NO: 41). For example, sortase B of S. aureus or B. anthracis cleaves the NPQTN (SEQ ID NO: 40) or NPKTG (SEQ ID NO: 41) motif of IsdC in the respective bacteria (see, e.g., Marraffimi, L. and Schneewind, O., Journal of Bacteriology, 189(17), p. 6425-6436, 2007). Other recognition motifs found in putative substrates of class B sortases are NSKTA (SEQ ID NO: 44), NPQTG (SEQ ID NO: 45), NAKTN (SEQ ID NO: 46), and NPQSS (SEQ ID NO: 47). For example, SrtB from L. monocytogenes recognizes certain motifs lacking P at position 2 and/or lacking Q or K at position 3, such as NAKTN (SEQ ID NO: 46) and NPQSS (SEQ ID NO: 47) (Mariscotti J F, Garcia-Del Portillo F, Pucciarelli M G. The Listeria monocytogenes sortase-B recognizes varied amino acids at position two of the sorting motif. J Biol Chem. 2009 Jan. 7. [Epub ahead of print])

In some embodiments, the sortase used for producing the conjugates described herein is class C sortase. Class C sortases may utilize LPXTG (SEQ ID NO: 25) as a recognition motif.

In some embodiments, the sortase is a class D sortase. Sortases in this class are predicted to recognize motifs with a consensus sequence NA-[E/A/S/H]-TG (Comfort D, supra). Class D sortases have been found, e.g., in Streptomyces spp., Corynebacterium spp., Tropheryma whipplei, Thermobifida fusca, and Bifidobacterium longhum. LPXTA (SEQ ID NO: 32) or LAXTG (SEQ ID NO: 48) may serve as a recognition sequence for class D sortases, e.g., of subfamilies 4 and 5, respectively subfamily-4 and subfamily-5 enzymes process the motifs LPXTA (SEQ ID NO: 32) and LAXTG (SEQ ID NO: 48), respectively). For example, B. anthracis Sortase C, which is a class D sortase, has been shown to specifically cleave the LPNTA (SEQ ID NO: 33) motif in B. anthracis BasI and BasH (Marrafini, supra).

In some embodiments, a variant of a naturally occurring sortase may be used. Such variants may be produced through processes such as directed evolution, site-specific modification, etc. For example, variants of S. aureus sortase A with up to a 140-fold increase in LPETG (SEQ ID NO: 29)-coupling activity compared with the starting wild-type enzyme have been identified (Chen, I., et al., PNAS 108(28): 11399-11404, 2011). In some embodiments a sortase variant comprises any one or more of the following substitutions relative to a wild type S. aureus SrtA: P94S or P94R, D160N, D165A, K190E, and K196T mutations. An exemplary wild type S. aureus SrtA sequence (Gene ID: 1125243, NCBI RefSeq Acc. No. NP_375640) is shown below:

(SEQ ID NO: 3)
MKKWTNRLMTIAGVVLILVAAYLFAKPHIDNYLHDKDKDEKIEQYDKNVK
EQASKDNKQQAKPQIPKDKSKVAGYIEIPDADIKEPVYPGPATPEQLNRG
VSFAEENESLDDQNISIAGHTFIDRPNYQFTNLKAAKKGSMVYFKVGNET
RKYKMTSIRDVKPTDVEVLDEQKGKDKQLTLITCDDYNEKTGVWEKRKIF
VATEVK

Sortase tagging can be used to install reactive chemistry moieties (e.g., click chemistry handles) onto a VHH, e.g., as described in U.S. Pat. No. 9,751,945, incorporated herein by reference. The click chemistry handle can be used for conjugating the VHH to other agents (e.g., antigens, anti-inflammatory agents, and/or proinflammatory agents). In some embodiments, the sortase recognition sequence is at the N-terminus of the VHH. In some embodiments, the sortase recognition sequence is at the C-terminus of the VHH.

In some embodiments, a reactive chemistry moiety is installed onto the VHH via a sortase mediated tagging (referred to as “sortagging”). Click chemistry handles are chemical moieties that provide a reactive group that can partake in a click chemistry reaction. Click chemistry reactions and suitable chemical groups for click chemistry reactions are well known to those of skill in the art, and include, but are not limited to terminal alkynes, azides, strained alkynes, dienes, dieneophiles, alkoxyamines, carbonyls, phosphines, hydrazides, thiols, and alkenes. For example, in some embodiments, an azide and an alkyne are used in a click chemistry reaction. Additional click chemistry handles suitable for use in the methods of protein conjugation described herein are well known to those of skill in the art, and such click chemistry handles include, but are not limited to, the click chemistry reaction partners, groups, and handles described in [1] H. C. Kolb, M. G. Finn, K. B. Sharpless, Angew. Chem. 2001, 113, 2056-2075; Angew. Chem. Int. Ed. 2001, 40, 2004-2021. [2] a) C. J. Hawker, K. L. Wooley, Science 2005, 309, 1200-1205; b) D. Fournier, R. Hoogenboom, U. S. Schubert, Chem. Soc. Rev. 2007, 36, 1369-1380; c) W. H. Binder, R. Sachsenhofer, Macromol. Rapid Commun. 2007, 28, 15-54; d) H. C. Kolb. K. B. Sharpless, Drug Discovery Today 2003, 8, 1128-1137; e) V. D. Bock, H. Hiemstra, J. H. van Maarseveen, Eur. J. Org. Chem. 2006, 51-68. [3] a) V. O. Rodionov, V. V. Fokin, M. G. Finn, Angew. Chem. 2005, 117, 2250-2255; Angew. Chem. Int. Ed. 2005, 44, 2210-2215; b) P. L. Golas, N. V. Tsarevsky, B. S. Sumerlin, K. Matyjaszewski, Macromolecules 2006, 39, 6451-6457; c) C. N. Urbani, C. A. Bell, M. R. Whittaker, M. J. Monteiro, Macromolecules 2008, 41, 1057-1060; d) S. Chassaing, A. S. S. Sido, A. Alix, M. Kumarraja, P. Pale, J. Sommer, Chem. Eur. J. 2008, 14, 6713-6721; e) B. C. Boren, S. Narayan, L. K. Rasmussen, L. Zhang, H. Zhao, Z. Lin, G. Jia, V. V. Fokin, J. Am. Chem. Soc. 2008, 130, 8923-8930; f) B. Saba, S. Sharma, D. Sawant, B. Kundu, Synlett 2007, 1591-1594. [4] J. F. Lutz, Angew. Chem. 2008, 120, 2212-2214; Angew. Chem. Int. Ed. 2008, 47, 2182-2184. [5] a) Q. Wang, T. R. Chan, R. Hilgraf, V. V. Fokin, K. B. Sharpless, M. G. Finn, J. Am. Chem. Soc. 2003, 125, 3192-3193; b) J. Gierlich, G. A. Burley, P. M. E. Gramlich, D. M. Hammond, T. Carell, Org. Lett. 2006, 8, 3639-3642. [6] a) J. M. Baskin, J. A. Prescher, S. T. Laughlin, N. J. Agard, P. V. Chang, I. A. Miller, A. Lo, J. A. Codelli, C. R. Bertozzi, Proc. Natl. Acad. Sci. USA 2007, 104, 16793-16797; b) S. T. Laughlin, J. M. Baskin, S. L. Amacher, C. R. Bertozzi, Science 2008, 320, 664-667; c) J. A. Johnson, J. M. Baskin, C. R. Bertozzi, J. F. Koberstein, N. J. Turro, Chem. Commun. 2008, 3064-3066; d) J. A. Codelli, J. M. Baskin, N. J. Agard, C. R. Bertozzi, J. Am. Chem. Soc. 2008, 130, 11486-11493; e) E. M. Sletten, C. R. Bertozzi, Org. Lett. 2008, 10, 3097-3099; f) J. M. Baskin, C. R. Bertozzi, QSAR Comb. Sci. 2007, 26, 1211-1219. [7] a) G. Wittig, A. Krebs, Chem. Ber. Reel. 1961, 94, 3260-3275; b) A. T. Blomquist, L. H. Liu, J. Am. Chem. Soc. 1953, 75, 2153-2154. [8] D. H. Ess, G. O. Jones, K. N. Houk, Org. Lett. 2008, 10, 1633-1636. [9] W. D. Sharpless, P. Wu, T. V. Hansen, J. G. Lindberg, J. Chem. Educ. 2005, 82, 1833-1836. [10] Y. Zou, J. Yin, Bioorg. Med. Chem. Lett. 2008, 18, 5664-5667. [11] X. Ning, J. Guo, M. A. Wolfert, G. J. Boons, Angew, Chem. 2008, 120, 2285-2287; Angew. Chem. Int. Ed. 2008, 47, 2253-2255. [12] S. Sawoo, P. Dutta, A. Chakraborty, R. Mukhopadhyay, O. Bouloussa, A. Sarkar, Chem. Commun. 2008, 5957-5959. [13] a) Z. Li, T. S. Seo, J. Ju, Tetrahedron Lett. 2004, 45, 3143-3146; b) S. S. van Berkel, A. J. Dirkes, M. F. Debets, F. L. van Delft, J. J. L. Cornelissen, R. J. M. Nolte, F. P. J. Rutjes, ChemBioChem 2007, 8, 1504-1508; c) S. S. van Berkel, A. J. Dirks, S. A. Meeuwissen, D. L. L. Pingen, O. C. Boerman, P. Laverman, F. L. van Delft, J. J. L. Cornelissen, F. P. J. Rutjes, ChemBioChem 2008, 9, 1805-1815. [14] F. Shi, J. P. Waldo, Y. Chen, R. C. Larock, Org. Lett. 2008, 10, 2409-2412. [15] L. Campbell-Verduyn, P. H. Elsinga, L. Mirfeizi, R. A. Dierckx, B. L. Feringa, Org. Biomol. Chem. 2008, 6, 3461-3463. [16] a) The Chemistry of the Thiol Group (Ed.: S. Patai), Wiley, New York, 1974; b) A. F. Jacobine, In Radiation Curing in Polymer Science and Technology III (Eds.: J. D. Fouassier, J. F. Rabek), Elsevier, London, 1993, Chap. 7, pp. 219-268. [17] C. E. Hoyle, T. Y. Lee, T. Roper, J. Polym. Sci. Part A 2008, 42, 5301-5338. [18] L. M. Campos, K. L. Killops, R. Sakai, J. M. J. Paulusse, D. Damiron, E. Drockenmuller, B. W. Messmore, C. J. Hawker, Macromolecules 2008, 41, 7063-7070. [19] a) R. L. A. David, J. A. Kornfield, Macromolecules 2008, 41, 1151-1161; b) C. Nilsson, N. Simpson, M. Malkoch, M. Johansson, E. Malmstrom, J. Polym. Sci. Part A 2008, 46, 1339-1348; c) A. Dondoni, Angew. Chem. 2008, 120, 9133-9135; Angew. Chem. Int. Ed. 2008, 47, 8995-8997; d) J. F. Lutz, H. Schlaad, Polymer 2008, 49, 817-824. [20] A. Gress, A. Voelkel, H. Schlaad, Macromolecules 2007, 40, 7928-7933. [21] N. ten Brummelhuis, C. Diehl, H. Schlaad, Macromolecules 2008, 41, 9946-9947. [22] K. L. Killops, L. M. Campos, C. J. Hawker, J. Am. Chem. Soc. 2008, 130, 5062-5064. [23] J. W. Chan, B. Yu, C. E. Hoyle, A. B. Lowe, Chem. Commun. 2008, 4959-4961. [24] a) G. Moad, E. Rizzardo, S. H. Thang, Ace. Chem. Res. 2008, 41, 1133-1142; b) C. Barner-Kowollik, M. Buback, B. Charleux, M. L. Coote, M. Drache, T. Fukuda, A. Goto, B. Klumperman, A. B. Lowe, J. B. McLeary, G. Moad, M. J. Monterio, R. D. Sanderson, M. P. Tonge, P. Vana, J. Polym. Sci. Part A 2006, 44, 5809-5831. [25] a) R. J. Pounder, M. J. Stanford, P. Brooks, S. P. Richards, A. P. Dove, Chem. Commun. 2008, 5158-5160; b) M. J. Stanford, A. P. Dove, Macromolecules 2009, 42, 141-147. [26] M. Li, P. De, S. R. Gondi, B. S. Sumerlin, J. Polym. Sci. Part A 2008, 46, 5093-5100. [27] Z. J. Witczak, D. Lorchak, N. Nguyen, Carbohydr. Res. 2007, 342, 1929-1933. [28] a) D. Samaroo, M. Vinodu, X. Chen, C. M. Drain, J. Comb. Chem. 2007, 9, 998-1011; b) X. Chen, D. A. Foster, C. M. Drain, Biochemistry 2004, 43, 10918-10929; c) D. Samaroo, C. E. Soll, L. J. Todaro, C. M. Drain, Org. Lett. 2006, 8, 4985-4988. [29] P. Battioni, O. Brigaud, H. Desvaux, D. Mansuy, T. G. Traylor, Tetrahedron Lett. 1991, 32, 2893-2896. [30] C. Ott, R. Hoogenboom, U. S. Schubert, Chem. Commun. 2008, 3516-3518. [31] a) V. Ladmiral, G. Mantovani, G. J. Clarkson, S. Cauet, J. L. Irwin, D. M. Haddleton, J. Am. Chem. Soc. 2006, 128, 4823-4830; b) S. G. Spain, M. I. Gibson, N. R. Cameron, J. Polym. Sci. Part A 2007, 45, 2059-2072. [32] C. R. Becer, K. Babiuch, K. Pilz, S. Hornig, T. Heinze, M. Gottschaldt, U. S. Schubert, Macromolecules 2009, 42, 2387-2394. [33] Otto Paul Hermann Diets and Kurt Alder first documented the reaction in 1928. They received the Nobel Prize in Chemistry in 1950 for their work on the eponymous reaction. [34] a) H. L. Holmes, R. M. Husband, C. C. Lee, P. Kawulka, J. Am. Chem. Soc. 1948, 70, 141-142; b) M. Lautens, W. Klute, W. Tarn, Chem. Rev. 1996, 96, 49-92; c) K. C. Nicolaou, S. A. Snyder, T. Montagnon, G. Vassilikogiannakis, Angew. Chem. 2002, 114, 1742-1773; Angew. Chem. Int. Ed. 2002, 41, 1668-1698; d) E. J. Corey, Angew. Chem. 2002, 114, 1724-1741; Angew. Chem. Int. Ed. 2002, 41, 1650-1667. [35] a) H. Durmaz, A. Dag, O. Altintas, T. Erdogan, G. Hizal, U. Tunca, Macromolecules 2007, 40, 191-198; b) H. Durmaz, A. Dag, A. Hizal, G. Hizal, U. Tunca, J. Polym. Sci. Part A 2008, 46, 7091-7100; c) A. Dag, H. Durmaz, E. Demir, G. Hizal, U. Tunca, J. Polym. Sci. Part A 2008, 46, 6969-6977; d) B. Gacal, H. Akat, D. K. Balta, N. Arsu, Y. Yagci, Macromolecules 2008, 41, 2401-2405; e) A. Dag, H. Durmaz, U. Tunca, G. Hizal, J. Polym. Sci. Part A 2009, 47, 178-187. [36] M. L. Blackman, M. Royzen, J. M. Fox, J. Am. Chem. Soc. 2008, 130, 13518-13519. [37] It should be noted that trans-cyclooctene is the most reactive dienophile toward tetrazines and seven orders of magnitude more reactive than cis-cyclooctene. [38] N. K. Devaraj, R. Weissleder, S. A. Hilderbrand, Bioconjugate Chem. 2008, 19, 2297-2299. [39] W. Song, Y. Wang, J. Qu, Q. Lin, J. Am. Chem. Soc. 2008, 130, 9654-9655. [40] W. Song, Y. Wang, J. Qu, M. M. Madden, Q. Lin, Angew. Chem. 2008, 120, 2874-2877; Angew. Chem. Int. Ed. 2008, 47, 2832-2835. [41] A. Dag, H. Durmaz, G. Hizal, U. Tunca, J. Polym. Sci. Part A 2008, 46, 302-313. [42] a) A. J. Inglis, S. Sinnwell, T. P. Davis, C. Barner-Kowollik, M. H. Stenzel, Macromolecules 2008, 41, 4120-4126; b) S. Sinnwell, A. J. Inglis, T. P. Davis, M. H. Stenzel, C. Barner-Kowollik, Chem. Commun. 2008, 2052-2054. [43] A. J. Inglis, S. Sinwell, M. H. Stenzel, C. Barner-Kowollik, Angew. Chem. 2009, 121, 2447-2450; Angew. Chem. Int. Ed. 2009, 48, 2411-2414. All references cited above are incorporated herein by reference for disclosure of click chemistry handles suitable for installation on proteins according to inventive concepts and methods provided herein.

Other tags can also be added to the VHH via sortagging. Examples of suitable tags include, without limitation, amino acids, peptides, proteins, nucleic acids, polynucleotides, sugars, carbohydrates, polymers, lipids, fatty acids, and small molecules. Other suitable tags will be apparent to those of skill in the art and the invention is not limited in this aspect. In some embodiments, a tag comprises a sequence useful for purifying, expressing, solubilizing, and/or detecting a polypeptide. In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a tag comprises an His6, HA, TAP, Myc, Flag, or GST tag, to name few examples. In some embodiments a tag comprises a solubility-enhancing tag (e.g., a SUMO tag, NUS A tag, SNUT tag, a Strep tag, or a monomeric mutant of the Ocr protein of bacteriophage T7). See, e.g., Esposito D and Chatterjee D K. Curr Opin Biotechnol.; 17(4):353-8 (2006). In some embodiments, a tag is cleavable, so that it can be removed, e.g., by a protease. In some embodiments, this is achieved by including a protease cleavage site in the tag, e.g., adjacent or linked to a functional portion of the tag. Exemplary proteases include, e.g., thrombin, TEV protease, Factor Xa, PreScission protease, etc. In some embodiments, a “self-cleaving” tag is used. See, e.g., PCT/US05/05763.

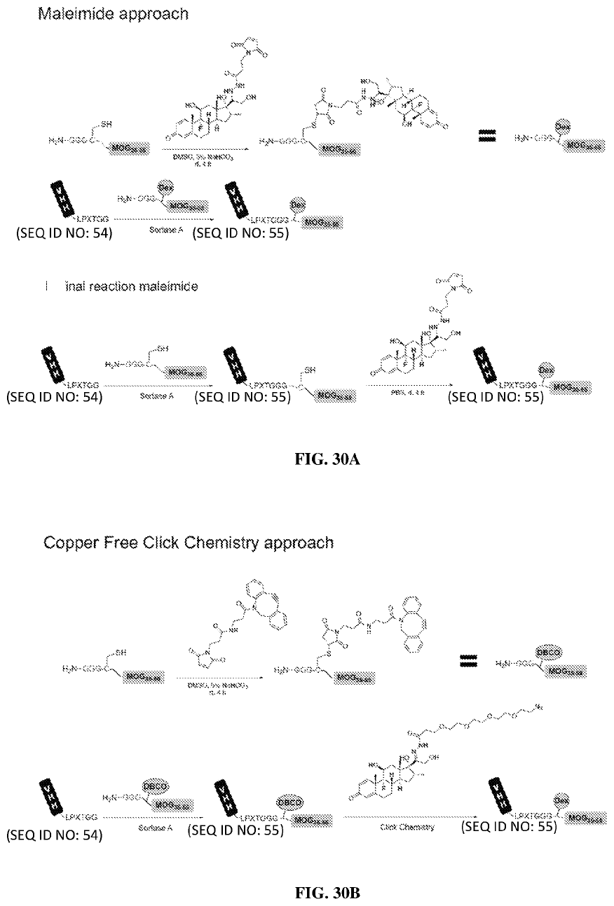

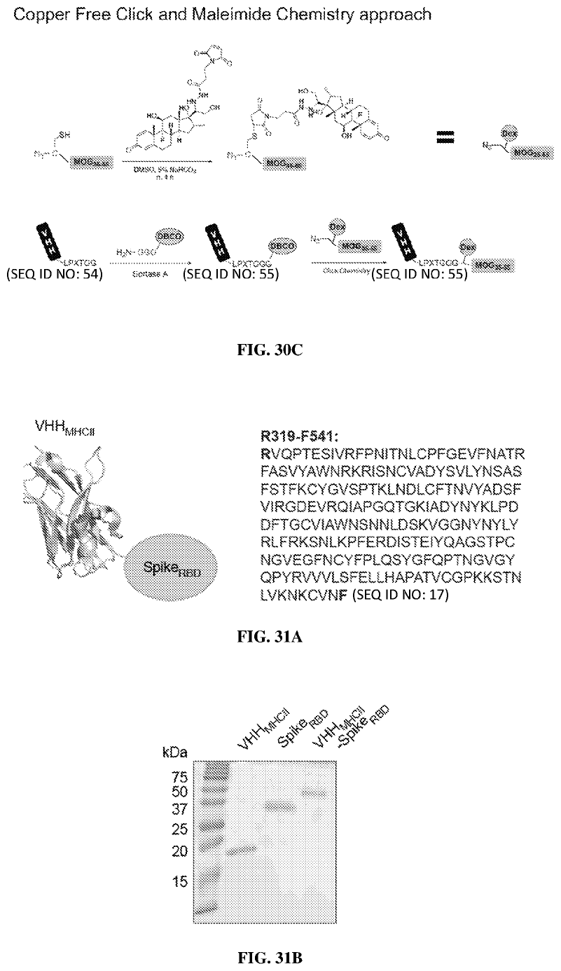

The conjugates described herein comprises a VHH conjugated to a second molecule. In some embodiments, the VHH comprises a sortase recognition motif and is conjugated to the second molecule via click chemistry. In some embodiments, the conjugate of the present disclosure comprises a VHH conjugated to one molecule. In some embodiments, the one molecule conjugated to the VHH is an antigen. In some embodiments, the one molecule conjugated to the VHH is an anti-inflammatory agent or a proinflammatory agent. In some embodiments, the conjugate of the present disclosure comprises a VHH conjugated to two molecules. In some embodiments, the conjugate of the present disclosure comprises a VHH conjugated to an antigen to which immune response is needed (e.g., an antigen from a pathogen or a tumor antigen) and an anti-inflammatory agent. In some embodiments, the conjugate of the present disclosure comprises a VHH conjugated to an antigen to which immune tolerance is needed (e.g., a self-antigen or an exogenous enzyme used for therapy) and a proinflammatory agent. Examples of methods for conjugating two molecules to a VHH are shown in FIGS. 30A-30C.

In some embodiments, an anti-inflammatory agent or a proinflammatory agent is conjugated to the sortase recognition motif of a VHH via a linker. In some embodiments, a linker is a non-hydrolysable linker (i.e. non-cleavable). Non-limiting examples of non-hydrolysable linkers include N-succinimidyl-4-(N-maleimidomethyl)cyclohexane-1-carboxylate (SMCC), maleimidomethyl cyclohexane-1-carboxylate (MCC), maleimidocaproyl (MC), and derivatives thereof. In some embodiments, a linker is a hydrolysable linker (i.e. cleavable). Non-limiting examples of hydrolysable linkers include hydrazone, hydrazide, disulfide, 4-(4′-acetylphenoxy)butanoic acid (AcBut), N-succinimidyl-4-(2-pyridyldithio)pentanoate (SPP) and N-succinimidyl-4-(2-pyridyldithio)butyrate (SPDB), valine-citrulline (VC), valine-alanine (VA), phenylalanine-lysine (FK), and derivatives thereof. A hydrolysable linker may be a self-immolating linker (i.e. self-cleaving), such as a pH-sensitive linker (e.g., hydrazone). A pH-sensitive linker may be used, for example, to release an anti-inflammatory agent or a proinflammatory agent conjugated to a VHH upon a shift in acidity of the physiological environment, such as when the VHH is delivered to a desired destination (e.g., an APC or intracellular compartment thereof). In some embodiments, the linker is a self-hydrolyzing hydrazone linker as shown in FIG. 27. Additional linkers suitable for use in the methods described herein are well known to those of skill in the art and include, but are not limited to, those described in Jain, N., Smith, S. W., Ghone, S., & Tomczuk, B. Pharm Res, 2015, 32(11), 3526-3540, and Lu, J., Jiang, F., Lu, A., & Zhang, G. Int J Mol Sci, 2016, 17(4), 561, both of which are incorporated by reference herein.

In some embodiments, the composition described herein comprises a conjugate comprising a VHH conjugated to an antigen (e.g., an antigen to which immune tolerance is needed) and an anti-inflammatory agent (e.g., dexamethasone), wherein the VHH binds to MHCII (e.g., the VHH having the amino acid sequence of SEQ ID NO: 1). In some embodiments, the composition described herein comprises a conjugate comprising a VHH conjugated to a self-antigen and an anti-inflammatory agent (e.g., dexamethasone), wherein the VHH binds to MHCII (e.g., the VHH having the amino acid sequence of SEQ ID NO: 1). Any one of the self-antigens described herein may be used. In some embodiments, the self-antigen is myelin oligodendrocyte glycoprotein (MOG), or a fragment thereof (e.g., amino acids 35-55 of the MOG protein. In some embodiments, the self-antigen is citrullinated fibrinogen. In some embodiments, the self-antigen is insulin. In some embodiments, the composition described herein comprises a conjugate comprising a VHH conjugated to a protein used in a protein replacement therapy or a gene therapy (e.g., an enzyme such as Factor IX or Factor VIII or an adeno-associated virus (AAV) derived protein) and an anti-inflammatory agent (e.g., dexamethasone), wherein the VHH binds to MHCII (e.g., the VHH having the amino acid sequence of SEQ ID NO: 1).

In some embodiments, the composition comprises a first conjugate comprising a first VHH conjugated to an antigen (e.g., an antigen to which immune tolerance is needed) and a second conjugate comprising a second VHH conjugated to an anti-inflammatory agent (e.g., dexamethasone), wherein the first VHH and the second VHH both bind to MHCII (e.g., the VHH having the amino acid sequence of SEQ ID NO: 1). In some embodiments, the composition comprises a first conjugate comprising a first VHH conjugated to a self-antigen and a second conjugate comprising a second VHH conjugated to an anti-inflammatory agent (e.g., dexamethasone), wherein the first VHH and the second VHH both bind to MHCII (e.g., the VHH having the amino acid sequence of SEQ ID NO: 1). Any one of the self-antigens described herein may be used. In some embodiments, the self-antigen is myelin oligodendrocyte glycoprotein (MOG), or a fragment thereof (e.g., amino acids 35-55 of the MOG protein. In some embodiments, the self-antigen is citrullinated fibrinogen. In some embodiments, the self-antigen is insulin. In some embodiments, the composition comprises a first conjugate comprising a first VHH conjugated to a protein used in a protein replacement therapy or a gene therapy (e.g., an enzyme such as Factor IX or Factor VIII or an adeno-associated virus (AAV) derived protein) and a second conjugate comprising a second VHH conjugated to an anti-inflammatory agent (e.g., dexamethasone), wherein the first VHH and the second VHH both bind to MHCII (e.g., the VHH having the amino acid sequence of SEQ ID NO: 1).

In some embodiments, the composition described herein comprises a conjugate comprising a VHH conjugated to an antigen (e.g., an antigen to which immune tolerance is needed) and an anti-inflammatory agent (e.g., dexamethasone), wherein the VHH binds to CD11c (e.g., the VHH having the amino acid sequence of SEQ ID NO: 2). In some embodiments, the composition described herein comprises a conjugate comprising a VHH conjugated to a self-antigen and an anti-inflammatory agent (e.g., dexamethasone), wherein the VHH binds to CD11c (e.g., the VHH having the amino acid sequence of SEQ ID NO: 2). Any one of the self-antigens described herein may be used. In some embodiments, the self-antigen is myelin oligodendrocyte glycoprotein (MOG), or a fragment thereof (e.g., amino acids 35-55 of the MOG protein. In some embodiments, the self-antigen is citrullinated fibrinogen. In some embodiments, the self-antigen is insulin. In some embodiments, the composition described herein comprises a conjugate comprising a VHH conjugated to a protein used in a protein replacement therapy or a gene therapy (e.g., an enzyme such as Factor IX or Factor VIII or an adeno-associated virus (AAV) derived protein) and an anti-inflammatory agent (e.g., dexamethasone), wherein the VHH binds to CD11c (e.g., the VHH having the amino acid sequence of SEQ ID NO: 2).

In some embodiments, the composition comprises a first conjugate comprising a first VHH conjugated to an antigen (e.g., an antigen to which immune tolerance is needed) and a second conjugate comprising a second VHH conjugated to an anti-inflammatory agent (e.g., dexamethasone), wherein the first VHH and the second VHH both bind to CD11c (e.g., the VHH having the amino acid sequence of SEQ ID NO: 2). In some embodiments, the composition comprises a first conjugate comprising a first VHH conjugated to a self-antigen and a second conjugate comprising a second VHH conjugated to an anti-inflammatory agent (e.g., dexamethasone), wherein the first VHH and the second VHH both bind to CD11c (e.g., the VHH having the amino acid sequence of SEQ ID NO: 2). Any one of the self-antigens described herein may be used. In some embodiments, the self-antigen is myelin oligodendrocyte glycoprotein (MOG), or a fragment thereof (e.g., amino acids 35-55 of the MOG protein. In some embodiments, the self-antigen is citrullinated fibrinogen. In some embodiments, the self-antigen is insulin. In some embodiments, the composition comprises a first conjugate comprising a first VHH conjugated to a protein used in a protein replacement therapy or a gene therapy (e.g., an enzyme such as Factor IX or Factor VIII or an adeno-associated virus (AAV) derived protein) and a second conjugate comprising a second VHH conjugated to an anti-inflammatory agent (e.g., dexamethasone), wherein the first VHH and the second VHH both bind to CD11c (e.g., the VHH having the amino acid sequence of SEQ ID NO: 2).

In some embodiments, the composition comprises a first conjugate comprising a first VHH conjugated to an antigen (e.g., an antigen to which immune tolerance is needed) and a second conjugate comprising a second VHH conjugated to an anti-inflammatory agent (e.g., dexamethasone), wherein the first VHH and the second VHH bind to different surface proteins on the APC. In some embodiments, the composition comprises a first conjugate comprising a first VHH conjugated to an antigen (e.g., an antigen to which immune tolerance is needed) and a second conjugate comprising a second VHH conjugated to an anti-inflammatory agent (e.g., dexamethasone), wherein the first VHH binds to MHCII (e.g., VHH having the amino acid sequence of SEQ ID NO: 1) and the second VHH binds to CD11c (e.g., VHH having the amino acid sequence of SEQ ID NO: 2). In some embodiments, the composition comprises a first conjugate comprising a first VHH conjugated to an antigen (e.g., an antigen to which immune tolerance is needed) and a second conjugate comprising a second VHH conjugated to an anti-inflammatory agent (e.g., dexamethasone), wherein first VHH binds to DEC205 and the second VHH binds to MHCII (e.g., VHH having the amino acid sequence of SEQ ID NO: 1). In some embodiments, the composition comprises a first conjugate comprising a first VHH conjugated to a self-antigen and a second conjugate comprising a second VHH conjugated to an anti-inflammatory agent (e.g., dexamethasone), wherein the first VHH binds to MHCII (e.g., VHH having the amino acid sequence of SEQ ID NO: 1) and the second VHH binds to CD11c (e.g., VHH having the amino acid sequence of SEQ ID NO: 2). Any one of the self-antigens described herein may be used. In some embodiments, the self-antigen is myelin oligodendrocyte glycoprotein (MOG), or a fragment thereof (e.g., amino acids 35-55 of the MOG protein. In some embodiments, the self-antigen is citrullinated fibrinogen. In some embodiments, the self-antigen is insulin. In some embodiments, the composition comprises a first conjugate comprising a first VHH conjugated to a protein used in a protein replacement therapy or a gene therapy (e.g., an enzyme such as Factor IX or Factor VIII or an adeno-associated virus (AAV) derived protein) and a second conjugate comprising a second VHH conjugated to an anti-inflammatory agent (e.g., dexamethasone), wherein first VHH binds to DEC205 and the second VHH binds to MHCII (e.g., VHH having the amino acid sequence of SEQ ID NO: 1).

In some embodiments, the composition described herein comprises a conjugate comprising a VHH conjugated to an antigen (e.g., an antigen to which immune response is needed) and a proinflammatory agent, wherein the VHH binds to MHCII (e.g., the VHH having the amino acid sequence of SEQ ID NO: 1). In some embodiments, the composition described herein comprises a conjugate comprising a VHH conjugated to an antigen from a pathogen (e.g., a SARS-CoV-2 protein such as the spike protein) and a proinflammatory agent (e.g., IL2), wherein the VHH binds to MHCII (e.g., the VHH having the amino acid sequence of SEQ ID NO: 1). Any one of the antigens from pathogens described herein may be used. In some embodiments, the composition described herein comprises a conjugate comprising a VHH conjugated to a tumor antigen and a proinflammatory agent (e.g., IL2), wherein the VHH binds to MHCII (e.g., the VHH having the amino acid sequence of SEQ ID NO: 1). Any one of the tumor antigens described herein may be used.

In some embodiments, the composition comprises a first conjugate comprising a first VHH conjugated to an antigen (e.g., an antigen to which immune response is needed) and a second conjugate comprising a second VHH conjugated to a proinflammatory agent, wherein the first VHH and the second VHH both bind to MHCII (e.g., the VHH having the amino acid sequence of SEQ ID NO: 1). In some embodiments, the composition comprises a first conjugate comprising a first VHH conjugated to an antigen from a pathogen (e.g., a SARS-CoV-2 protein such as the spike protein) and a second conjugate comprising a second VHH conjugated to a proinflammatory agent (e.g., IL2), wherein the first VHH and the second VHH both bind to MHCII (e.g., the VHH having the amino acid sequence of SEQ ID NO: 1). Any one of the antigens from pathogens described herein may be used. In some embodiments, the composition comprises a first conjugate comprising a first VHH conjugated to a tumor antigen and a second conjugate comprising a second VHH conjugated to a proinflammatory agent (e.g., IL2), wherein the first VHH and the second VHH both bind to MHCII (e.g., the VHH having the amino acid sequence of SEQ ID NO: 1). Any one of the tumor antigens described herein may be used.

In some embodiments, the composition described herein comprises a conjugate comprising a VHH conjugated to an antigen (e.g., an antigen to which immune response is needed) and a proinflammatory agent, wherein the VHH binds to CD11c (e.g., the VHH having the amino acid sequence of SEQ ID NO: 2). In some embodiments, the composition described herein comprises a conjugate comprising a VHH conjugated to an antigen from a pathogen (e.g., a SARS-CoV-2 protein such as the spike protein) and a proinflammatory agent (e.g., IL2), wherein the VHH binds to CD11c (e.g., the VHH having the amino acid sequence of SEQ ID NO: 2). Any one of the antigens from pathogens described herein may be used. In some embodiments, the composition described herein comprises a conjugate comprising a VHH conjugated to a tumor antigen and a proinflammatory agent (e.g., IL2), wherein the VHH binds to CD11c (e.g., the VHH having the amino acid sequence of SEQ ID NO: 2).

In some embodiments, the composition comprises a first conjugate comprising a first VHH conjugated to an antigen (e.g., an antigen to which immune response is needed) and a second conjugate comprising a second VHH conjugated to a proinflammatory agent, wherein the first VHH and the second VHH both bind to CD11c (e.g., the VHH having the amino acid sequence of SEQ ID NO: 2). In some embodiments, the composition comprises a first conjugate comprising a first VHH conjugated to an antigen from a pathogen (e.g., a SARS-CoV-2 protein such as the spike protein) and a second conjugate comprising a second VHH conjugated to a proinflammatory agent (e.g., IL2), wherein the first VHH and the second VHH both bind to CD11c (e.g., the VHH having the amino acid sequence of SEQ ID NO: 2). Any one of the antigens from pathogens described herein may be used. In some embodiments, the composition comprises a first conjugate comprising a first VHH conjugated to a tumor antigen and a second conjugate comprising a second VHH conjugated to a proinflammatory agent (e.g., 112), wherein the first VHH and the second VHH both bind to CD11c (e.g., the VHH having the amino acid sequence of SEQ ID NO: 2). Any one of the tumor antigens described herein may be used.

In some embodiments, the composition comprises a first conjugate comprising a first VHH conjugated to an antigen (e.g., an antigen to which immune response is needed) and a second conjugate comprising a second VHH conjugated to a proinflammatory agent, wherein the first VHH and the second VHH bind to different surface proteins on the APC. In some embodiments, the composition comprises a first conjugate comprising a first VHH conjugated to an antigen (e.g., an antigen to which immune response is needed) and a second conjugate comprising a second VHH conjugated to a proinflammatory agent (e.g., IL2), wherein the first VHH binds to MHCII (e.g., VHH having the amino acid sequence of SEQ ID NO: 1) and the second VHH binds to CD11c (e.g., VHH having the amino acid sequence of SEQ ID NO: 2). In some embodiments, the composition comprises a first conjugate comprising a first VHH conjugated to an antigen (e.g., an antigen to which immune response is needed) and a second conjugate comprising a second VHH conjugated to a proinflammatory agent (e.g., IL2), wherein first VHH binds to DEC205 and the second VHH binds to MHCII (e.g., VHH having the amino acid sequence of SEQ ID NO: 1). In some embodiments, the composition comprises a first conjugate comprising a first VHH conjugated to an antigen from a pathogen (e.g., a SARS-CoV-2 protein such as the spike protein) and a second conjugate comprising a second VHH conjugated to a proinflammatory agent (e.g., IL2), wherein the first VHH binds to MHCII (e.g., VHH having the amino acid sequence of SEQ ID NO: 1) and the second VHH binds to CD11c (e.g., VHH having the amino acid sequence of SEQ ID NO: 2). Any one of the antigens from pathogens described herein may be used. In some embodiments, the composition comprises a first conjugate comprising a first VHH conjugated to a tumor antigen and a second conjugate comprising a second VHH conjugated to a proinflammatory agent (e.g., IL2), wherein first VHH binds to DEC205 and the second VHH binds to MHCII (e.g., VHH having the amino acid sequence of SEQ ID NO: 1).

### Antigens

An “antigen,” as used herein, refers to a molecule that induces an immune response in a subject. An antigen of interest may be or may comprise, for example, a polypeptide, a polysaccharide, a carbohydrate, a lipid, a nucleic acid, or combination thereof. An antigen may be naturally occurring or synthetic.

In some embodiments, an antigen is an antigen to which immune tolerance is needed. In some embodiments, such an antigen is a self-antigen (also referred to as “autoantigen”) or an agent that has the capacity to initiate or enhance an autoimmune response, causing autoimmune diseases. It is thus desired to induce immune tolerance to such self-antigens. In some embodiments, the compositions described herein are used to induce immune tolerance (e.g., antigen specific immune tolerance) to self-antigens. Induction of immune tolerance (e.g., antigen specific immune tolerance) reduces antigen-specific immune responses to the antigen, which, in some embodiments, alleviates the severity of autoimmune diseases.

In some embodiments, the self-antigen used in accordance with the present disclosure is selected from the group consisting of: myelin oligodendrocyte glycoprotein (MOG), myelin proteolipid protein, citrullinated fibrinogen, insulin, chromogranin A, GAD65, desmoglein 1 (DSG1) and desmoglein 3 (DSG3), acetylcholine receptor (AChR), muscle-specific tyrosine kinase (MuSK), and ribonucleoproteins.

In some embodiments, the self-antigen comprises myelin oligodendrocyte glycoprotein (MOG) or an antigenic fragment thereof. Myelin oligodendrocyte glycoprotein (MOG) is a membrane-embedded surface protein of the central nervous system (CNS) myelin sheath. Antibodies targeting MOG have been consistently found in the sera of patients suffering from autoimmune diseases such as acquired inflammatory demyelinating disorders of the CNS (e.g., as described in Nessier et al., EBioMedicine. 2019 October; 48: 18-19, incorporated herein by reference). Autoimmune diseases associated with MOG antibodies include, without limitation, acute disseminated encephalomyelitis (ADEM), optic neuritis (ON), transverse myelitis and brainstem encephalitis. In some embodiments, the self-antigen in the composition described herein is full length MOG. In some embodiments, the self-antigen in the composition described herein comprises a MOG fragment (e.g., amino acids 35-55 of MOG, MEVGWYRSPFSRVVHLYRNGK (SEQ ID NO: 49)).

In some embodiments, the self-antigen comprises fibrinogen or an antigenic fragment thereof. Fibrinogen (coagulation factor 1) is a major player in thrombus formation; it is cleaved by thrombin to form fibrin, which is the most abundant component of a blood clot. Fibrinogen plays an important role in coagulation and cardiovascular diseases (CVDs). Additionally, fibrinogen is a proinflammatory factor in autoimmune and inflammatory diseases such as rheumatoid arthritis, vasculitides, inflammatory bowel disease, multiple sclerosis, chronic obstructive pulmonary diseases, kidney disorders, and posttransplantation fibrosis and in several types of cancer (e.g., as described in Arbustini et al., Circulation. 2013; 128:1276-1280, incorporated herein by reference). In some embodiments, the self-antigen is citrullinated fibrinogen. In some embodiments, the self-antigen in the composition described herein comprises a fibrinogen fragment (amino acids 79-91 of fibrinogen, citrullinated, QDFTNCitINKLKNS (SEQ ID NO: 50)). Anti-citrullinated protein antibodies (ACPA) are specifically and frequently detected in sera of patients with rheumatoid arthritis (e.g., as described in Takizawa et al., Ann Rheum Dis. 2006 August; 65(8): 1013-1020).

In some embodiments, the self-antigen comprises myelin proteolipid protein or an antigenic fragment thereof. Myelin proteolipid protein has been shown to be involved in autoimmune demyelinating disease, e.g., as described in Tuohy et al., Neurochem Res. 1994 August; 19(8):935-44, incorporated herein by reference.

In some embodiments, the self-antigen comprises insulin or an antigenic fragment thereof. In some embodiments, the self-antigen comprises insulin alpha chain GIVEQCCTSICSLYQLENYCN (SEQ ID NO: 51)). In some embodiments, the self-antigen comprises insulin beta chain FVNQHLCGSHLVEALYLVCGERGFFYTPKT (SEQ ID NO: 52)). Insulin is involved in rare autoimmune diseases including insulin autoimmune syndrome and type B insulin resistance syndrome (e.g., as described in Censi et al., Ann Transl Med. 2018 September; 6(17): 335, incorporated herein by reference).

In some embodiments, the self-antigen comprises chromogranin A or an antigenic fragment thereof. Chromogranin A is associated with autoimmune gastritis (e.g., as described in Peracchi et al., European Journal of Endocrinology (2005) 152 443-448, incorporated herein by reference).

In some embodiments, the self-antigen comprises glutamic acid decarboxylase 65-kilodalton isoform (GAD65) or an antigenic fragment thereof, which is known to be associated with autoimmune diseases of the central nervous system, neurological autoimmune diseases. Type 1 diabetes, autoimmune thyroid disease, and pernicious anemia (e.g., as described in McKeon et al., Muscle Nerve. 2017 July; 56(1):15-27, incorporated herein by reference).

In some embodiments, the self-antigen comprises desmoglein 1 (DSG1) and/or desmoglein 3 (DSG3) or an antigenic fragment thereof. DSG1 and DSG3 are involved in skin autoimmune disease, e.g., as described in Amagai et al., Proc Jpn Acad Ser B Phys Biol Sci. 2010; 86(5):524-37, incorporated herein by reference.

In some embodiments, the self-antigen comprises acetylcholine receptor (AChR) or an antigenic fragment thereof. Antibody-mediated autoimmune response to acetylcholine receptor causes myasthenia gravis, e.g., as described in Lindstrom et al., J Neurobiol. 2002 December; 53(4):656-65, incorporated herein by reference.

In some embodiments, the self-antigen comprises muscle-specific tyrosine kinase (MuSK) or an antigenic fragment thereof. MuSK has been shown to be involved in neuromuscular junction autoimmune diseases, e.g., as described in Vincent et al., Cuff Opin Neurol. 2005 October; 18(5):519-25, incorporated herein by reference.

In some embodiments, the self-antigen comprises a ribonucleoprotein or an antigenic fragment thereof. Ribonucleoproteins are involved in autoimmune diseases such as Systemic Lupus Erythematosus (SLE) and Mixed connective tissue disease (MCTD), e.g., as described in Whittingham et al., Aust N Z J Med. 1983 December; 13(6):565-70; and Newkirk et al., Arthritis Research & Therapy volume 3, Article number: 253 (2001), incorporated herein by reference.

Other non-limiting examples of such autoimmune antigens and associated autoimmune diseases include: pancreatic beta-cell antigens, insulin and GAD to treat insulin-dependent diabetes mellitus (type I diabetes); collagen type 11, human cartilage gp39 (HCgp39) and gpl30-RAPS for use in treating rheumatoid arthritis; myelin basic protein (MBP), proteolipid protein (PLP) to treat multiple sclerosis; fibrillarin, and small nucleolar protein (snoRNP) to treat scleroderma; thyroid stimulating factor receptor (TSH-R) for use in treating Graves' disease; nuclear antigens, histones, glycoprotein gp70 and ribosomal proteins for use in treating systemic lupus erythematosus; pyruvate dehydrogenase dehydrolipoamide acetyltransferase (PCD-E2) for use in treating primary biliary cirrhosis; hair follicle antigens for use in treating alopecia areata; and human tropomyosin isoform 5 (hTM5) for use in treating ulcerative colitis. These examples are not meant to be limiting. One skilled in the art is able to identify the autoimmune antigen associated with the autoimmune disease of interest.

In some embodiments, the antigen comprises a protein used in a protein replacement therapy or a gene therapy, e.g., without limitation, Factor IX, Factor VIII, insulin, and AAV-derived proteins. These examples are not meant to be limiting. One skilled in the art is able to identify the proteins of interest that are used in protein replacement therapies or gene therapies. Inducing immune tolerance against these proteins reduces the destruction of the proteins by the immune system, leading to longer lasting therapeutic effect.

In some embodiments, the antigen used in accordance with the present disclosure is an antigen to which immune response is needed. For example, in some embodiments, such antigen is naturally produced by and/or comprises a polypeptide or peptide that is genetically encoded by a pathogen, an infected cell, or a neoplastic cell (e.g., a cancer cell). In some embodiments, an antigen is produced or genetically encoded by a virus, bacteria, fungus, or parasite which, in some embodiments, is a pathogenic agent. In some embodiments, a pathogen is intracellular during at least part of its life cycle. In some embodiments, a pathogen is extracellular. It will be appreciated that an antigen that originates from a particular source may, in some embodiments, be isolated from such source, or produced using any appropriate means (e.g., recombinantly, synthetically, etc.), e.g., for purposes of using the antigen, e.g., to identify, generate, test, or use an antibody thereto). An antigen may be modified, e.g., by conjugation to another molecule or entity (e.g., an adjuvant), chemical or physical denaturation, etc. In some embodiments, an antigen is an envelope protein, capsid protein, secreted protein, structural protein, cell wall protein or polysaccharide, capsule protein or polysaccharide, or enzyme. In some embodiments an antigen is a toxin, e.g., a bacterial toxin. In some embodiments, the antigen is a viral antigen. Exemplary viruses include, e.g., SARS-CoV-2, Retroviridae (e.g., lentiviruses such as human immunodeficiency viruses, such as HIV-I); Caliciviridae (e.g. strains that cause gastroenteritis); Togaviridae (e.g. equine encephalitis viruses, rubella viruses); Flaviridae (e.g. dengue viruses, encephalitis viruses, yellow fever viruses, hepatitis C virus); Coronaviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), EBV, KSV); Poxviridae (variola viruses, vaccinia viruses, pox viruses); and Picornaviridae (e.g. polio viruses, hepatitis A virus; enteroviruses, human coxsackie viruses, rhinoviruses, echoviruses). In some embodiments, the antigen comprises a Beta Coronavirus protein such as the spike protein (e.g., full-length or receptor binding domain (RBD)), envelop protein, membrane protein, or nucleocapsid protein. In some embodiments, the antigen comprises a SARS-CoV (e.g., SARS-CoV-1 or SARS-CoV-2) protein such as the spike protein (e.g., full-length or receptor binding domain (RBD)), envelop protein, membrane protein, or nucleocapsid protein. Examples of Beta Coronavirus proteins that may be used as an antigen in accordance with the present disclosure are provided in Table 2.

TABLE 2
Beta Coronavirus protein antigens

| Antigen | Amino Acid Sequence |
|---|---|
| SARS-CoV-1 | MFIFLLFLTLTSGSDLDRCTTFDDVQAPNYTQHTSSMRGVYYPDEIFRSDTLYLTQDLFLPFY |
| Spike Protein | SNVTGFHTINHTFGNPVIPFKDGIYFAATEKSNVVRGWVFGSTMNNKSQSVIIINNSTNVVIR |
| (SEQ ID NO: | ACNFELCDNPFFAVSKPMGTQTHTMIFDNAFNCTFEYISDAFSLDVSEKSGNFKHLREFVFK |
| 10) | NKDGFLYVYKGYQPIDVVRDLPSGFNTLKPIFKLPLGINITNFRAILTAFSPAQDIWGTSAAAY |
| | FVGYLKPTTFMLKYDENGTITDAVDCSQNPLAELKCSVKSFEIDKGIYQTSNFRVVPSGDVVR |
| | FPNITNLCPFGEVFNATKFPSVYAWERKKISNCVADYSVLYNSTFFSTFKCYGVSATKLNDLC |
| | FSNVYADSFVVKGDDVRQIAPGQTGVIADYNYKLPDDFMGCVLAWNTRNIDATSTGNYNYK |
| | YRYLRHGKLRPFERDISNVPFSPDGKPCTPPALNCYWPLNDYGFYTTTGIGYQPYRVVVLSF |
| | ELLNAPATVCGPKLSTDLIKNQCVNFNFNGLTGTGVLTPSSKRFQPFQQFGRDVSDFTDSVR |
| | DPKTSEILDISPCSFGGVSVITPGTNASSEVAVLYQDVNCTDVSTAIHADQLTPAWRIYSTGN |
| | NVFQTQAGCLIGAEHVDTSYECDIPIGAGICASYHTVSLLRSTSQKSIVAYTMSLGADSSIAYSN |
| | NTIAIPTNFSISITTEVMPVSMAKTSVDCNMYICGDSTECANLLLQYGSFCTQLNRALSGIAAE |
| | QDRNTREVFAQVKQMYKTPTLKYFGGFNFSQILPDPLKPTKRSFIEDLLFNKVTLADAGFM |
| | KQYGECLGDINARDLICAQKFNGLTVLPPLLTDDMIAAYTAALVSGTATAGWTFGAGAALQI |
| | PFAMQMAYRFNGIGVTQNVLYENQKQIANQFNKAISQIQESLTTTSTALGKLQDVVNQNAQ |
| | ALNTLVKQLSSNFGAISSVLNDILSRLDKVEAEVQIDRLITGRLQSLQTYVTQQLIRAAEIRAS |
| | ANLAATKMSECVLGQSKRVDFCGKGYHLMSFPQAAPHGVVFLHVTYVPSQERNFTTAPAIC |
| | HEGKAYFPREGVFVFNGTSWFITQRNFFSPQIITTDNTFVSGNCDVVIGIINNTVYDPLQPEL |
| | DSFKEELDKYFKNHTSPDVDLGDISGINASVVNIQKEIDRLNEVAKNLNESLIDLQELGKYEQ |
| | YIKWPWYVWLGFIAGLIAIVMVTILLCCMTSCCSCLKGACSCGSCCKFDEDDSEPVLKGVKLH |
| | YT |
| SARS-CoV-1 | RVVPSGDVVRFPNITNLCPFGEVFNATKFPSVYAWERKKISNCVADYSVLYNSTFFSTFKCYG |
| Spike Protein | VSATKLNDLCFSNVYADSFVVKGDDVRQIAPGQTGVIADYNYKLPDDFMGCVLAWNTRNID |
| receptor | ATSTGNYNYKYRYLRHGKLRPFERDISNVPFSPDGKPCTPPALNCYWPLNDYGFYTTTGIGY |
| binding | QPYRVVVLSFELLNAPATVCGPKLSTDLIKNQCVNF |
| domain (SEQ | |
| ID NO: 11) | |
| SARS-CoV-1 | MYSFVSEETGTLIVNSVLLFLAFVVFLLVTLAILTALRLCAYCCNIVNVSLVKPTVYVYSRVKN |
| Envelope | LNSSEGVPDLLV |
| Protein (SEQ | |
| ID NO: 12) | |
| SARS-CoV-1 | MADNGTITVEELKQLLEQWNLVIGFLFLAWIMLLQFAYSNRNRFLYIIKLVFLWLLWPVTLA |
| Membrane | CFVLAAVYRINWVTGGIAIAMACIVGLMWLSYFVASFRLFARTRSMWSFNPETNILLNVPLR |
| Protein (SEQ | GTIVTRPLMESELVIGAVIIRGHLRMAGHSLGRCDIKDLPKEITVATSRTLSYYKLGASQRVGT |
| ID NO: 14) | DSGFAAYNRYRIGNYKLNTDHAGSNDNIALLVQ |
| SARS-CoV-1 | MSDNGPQSNQRSAPRITFGGPTDSTDNNQNGGRNGARPKQRRPQGLPNNTASWFTALTQH |
| Nucleocapsid | GKEELRFPRGQGVPINTNSGPDDQIGYYRRATRRVRGGDGKMKELSPRWYFYYLGTGPEAS |
| Protein (SEQ | LPYGANKEGIVWVATEGALNTPKDHIGTRNPNNNAATVLQLPQGTTLPKGFYAEGSRGGSQ |
| ID NO: 15) | ASSRSSSRSRGNSRNSTPGSSRGNSPARMASGGGETALALLLLDRLNQLESKVSGKGQQQQG |
| | QTVTKKSAAEASKKPRQKRTATKQYNVTQAFGRRGPEQTQGNFGDQDLIRQGTDYKHWPQ |
| | IAQFAPSASAFFGMSRIGMEVTPSGTWLTYHGAIKLDDKDPQFKDNVILLNKHIDAYKTFPP |
| | TEPKKDKKKKTDEAQPLPQRQKKQPTVTLLPAADMDDFSRQLQNSMSGASADSTQA |
| SARS-CoV-2 | MFVFLVLLPLVSSQCVNLTTRTQLPPAYTNSFTRGVYYPDKVFRSSVLHSTQDLFLPFFSNVT |
| Spike Protein | WFHAIHVSGTNGTKRFDNPVLPFNDGVYFASTEKSNIIRGWIFGTTLDSKTQSLLIVNNATN |
| (SEQ ID NO: | VVIKVCEFQFCNDPFLGVYYHKNNKSWMESEFRVYSSANNCTFEYVSQPFLMDLEGKQGNF |
| 16) | KNLREFVFKNIDGYFKIYSKHTPINLVRDLPQGFSALEPLVDLPIGINITRFQTLLALHRSYLT |
| | PGDSSSGWTAGAAAYYVGYLQPRTFLLKYNENGTITDAVDCALDPLSETKCTLKSFTVEKGI |
| | YQTSNFRVQPTESIVRFPNITNLCPFGEVFNATRFASVYAWNRKRISNCVADYSVLYNSASFS |
| | TFKCYGVSPTKLNDLCFTNVYADSFVIRGDEVRQIAPGQTGKIADYNYKLPDDFTGCVIAWN |
| | SNNLDSKVGGNYNYLYRLFRKSNLKPFERDISTEIYQAGSTPCNGVEGFNCYFPLQSYGFQPT |
| | NGVGYQPYRVVVLSFELLHAPATVCGPKKSTNLVKNKCVNFNFNGLTGTGVLTESNKKFLPF |
| | QQFGRDIADTTDAVRDPQTLEILDITPCSFGGVSVITPGTNTSNQVAVLYQDVNCTEVPVAIH |
| | ADQLTPTWRVYSTGSNVFQTRAGCLIGAEHVNNSYECDIPIGAGICASYQTQTNSPRRARSV |
| | ASQSIIAYTMSLGAENSVAYSNNSIAIPTNFTISVTTEILPVSMTKTSVDCTMYICGDSTECSNL |
| | LLQYGSFCTQLNRALTGIAVEQDKNTQEVFAQVKQIYKTPPIKDFGGFNFSQILPDPSKPSKR |
| | SFIEDLLFNKVTLADAGFIKQYGDCLGDIAARDLICAQKFNGLTVLPPLLTDEMIAQYTSALL |
| | AGTITSGWTFGAGAALQIPFAMQMAYRFNGIGVTQNVLYENQKLIANQFNSAIGKIQDSLSS |
| | TASALGKLQDVVNQNAQALNTLVKQLSSNFGAISSVLNDILSRLDKVEAEVQIDRLITGRLQS |
| | LQTYVTQQLIRAAEIRASANLAATKMSECVLGQSKRVDFCGKGYHLMSFPQSAPHGVVFLHV |
| | TYVPAQEKNFTTAPAICHDGKAHFPREGVFVSNGTHWFVTQRNFYEPQIITTDNTFVSGNC |
| | DWIGIVNNTVYDPLQPELDSFKEELDKYFKNHTSPDVDLGDISGINASWNIQKEIDRLNEV |
| | AKNLNESLIDLQELGKYEQYIKWPWYIWLGFIAGLIAIVMVTIMLCCMTSCCSCLKGCCSCGS |
| | CCKFDEDDSEPVLKGVKLHYT |
| SARS-CoV-2 | RVQPTESIVRFPNITNLCPFGEVFNATRFASVYAWNRKRISNCVADYSVLYNSASFSTFKCYG |
| Spike Protein | VSPTKLNDLCFTNVYADSFVIRGDEVRQIAPGQTGKIADYNYKLPDDFTGCVIAWNSNNLDS |
| receptor | KVGGNYNYLYRLFRKSNLKPFERDISTEIYQAGSTPCNGVEGFNCYFPLQSYGFQPTNGVGYQ |
| binding | PYRVVVLSFELLHAPATVCGPKKSTNLVKNKCVNF |
| domain (SEQ | |
| ID NO: 17) | |
| SARS-CoV-2 | MYSFVSEETGTLIVNSVLLFLAFVVFLLVTLAILTALRLCAYCCNIVNVSLVKPSFYVYSRVKN |
| Envelope | LNSSRVPDLLV |
| Protein (SEQ | |
| ID NO: 18) | |
| SARS-CoV-2 | MADSNGTITVEELKKLLEQWNLVIGFLFLTWICLLQFAYANRNRFLYIIKLIFLWLLWPVTL |
| Membrane | ACFVLAAVYRINWITGGIAIAMACLVGLMWLSYFIASFRLFARTRSMWSFNPETNILLNVPL |
| Protein (SEQ | HGTILTRPLLESELVIGAVILRGHLRIAGHHLGRCDIKDLPKEITVATSRTLSYYKLGASQRVA |
| ID NO: 19) | GDSGFAAYSRYRIGNYKLNTDHSSSSDNIALLVQ |
| SARS-CoV-2 | MSDNGPQNQRNAPRITFGGPSDSTGSNQNGERSGARSKQRRPQGLPNNTASWFTALTQHG |
| Nucleocapsid | KEDLKFPRGQGVPINTNSSPDDQIGYYRRATRRIRGGDGKMKDLSPRWYFYYLGTGPEAGLP |
| Protein (SEQ | YGANKDGIIWVATEGALNTPKDHIGTRNPANNAAIVLQLPQGTTLPKGFYAEGSRGGSQASS |
| ID NO: 20) | RSSSRSRNSSRNSTPGSSRGTSPARMAGNGGDAALALLLLDRLNQLESKMSGKGQQQQGQT |
| | VTKKSAAEASKKPRQKRTATKAYNVTQAFGRRGPEQTQGNFGDQELIRQGTDYKHWPQIA |
| | QFAPSASAFFGMSRIGMEVTPSGTWLTYTGAIKLDDKDPNFKDQVILLNKHIDAYKTFPPTE |
| | PKKDKKKKADETQALPQRQKKQQTVTLLPAADLDDFSKQLQQSMSSADSTQA |
| MERS-CoV | MIHSVFLLMFLLTPTESYVDVGPDSVKSACIEVDIQQTFFDKTWPRPIDVSKADGIIYPQGRT |
| Spike protein | YSNITITYQGLFPYQGDHGDMYVYSAGHATGTTPQKLFVANYSQDVKQFANGFVVRIGAAA |
| (SEQ ID NO: | NSTGTVIISPSTSATIRKIYPAFMLGSSVGNFSDGKMGRFFNHTLVLLPDGCGTLLRAFYCILE |
| 21) | PRSGNHCPAGNSYTSFATYHTPATDCSDGNYNRNASLNSFKEYFNLRNCTFMYTYNITEDEI |
| | LEWFGITQTAQGVHLFSSRYVDLYGGNMFQFATLPVYDTIKYYSIIPHSIRSIQSDRKAWAAF |
| | YVYKLQPLTFLLDFSVDGYIRRAIDCGFNDLSQLHCSYESFDVESGVYSVSSFEAKPSGSVVEQ |
| | AEGVECDFSPLLSGTPPQVYNFKRLVFTNCNYNLTKLLSLFSVNDFTCSQISPAAIASNCYSSL |
| | ILDYFSYPLSMKSDLSVSSAGPISQFNYKQSFSNPTCLILATVPHNLTTITKPLKYSYINKCSRL |
| | LSDDRTEVPQLVNANQYSPCVSTVPSTVWEDGDYYRKQLSPLEGGGWLVASGSTVAMTEQL |
| | QMGFGITVQYGTDTNSVCPKLEFANDTKIASQLGNCVEYSLYGVSGRGVFQNCTAVGVRQQR |
| | FVYDAYQNLVGYYSDDGNYYCLRACVSVPVSVIYDKETKTHATLFGSVACEHISSTMSQYSRS |
| | TRSMLKRRDSTYGPLQTPVGCVLGLVNSSLFVEDCKLPLGQSLCALPDTPSTLTPRSVRSVPG |
| | EMRLASIAFNHPIQVDQLNSSYFKLSIPTNFSFGVTQEYIQTTIQKVTVDCKQYVCNGFQKCE |
| | QLLREYGQFCSKINQALHGANLRQDDSVRNLFASVKSSQSSPIIPGFGGDFNLTLLEPVSISTG |
| | SRSARSAIEDLLFDKVTIADPGYMQGYDDCMQQGPASARDLICAQYVAGYKVLPPLMDVNM |
| | EAAYTSSLLGSIAGVGWTAGLSSFAAIPFAQSIFYRLNGVGITQQVLSENQKLIANKFNQALGA |
| | MQTGFTTTNEAFRKVQDAVNNNAQALSKLASELSNTFGAISASIGDIIQRLDVLEQDAQIDRL |
| | INGRLTTLNAFVAQQLVRSESAALSAQLAKDKVNECVKAQSKRSGFCGQGTHIVSFVVNAPN |
| | GLYFMHVGYYPSNHIEVVSAYGLCDAANPTNCIAPVNGYFIKTNNTRIVDEWSYTGSSFYAP |
| | EPITSLNTKYVAPQVTYQNISTNLPPPLLGNSTGIDFQDELDEFFKNVSTSIPNFGSLTQINTT |
| | LLDLTYEMLSLQQVVKALNESYIDLKELGNYTYYNKWPWYIWLGFIAGLVALALCVFFILCC |
| | TGCGTNCMGKLKCNRCCDRYEEYDLEPHKVHVH |
| MERS-CoV | ECDFSPLLSGTPPQVYNFKRLVFTNCNYNLTKLLSLFSVNDFTCSQISPAAIASNCYSSLILDYF |
| Spike protein | SYPLSMKSDLSVSSAGPISQFNYKQSFSNPTCLILATVPHNLTTITKPLKYSYINKC |
| receptor | |
| binding | |
| domain (SEQ | |
| ID NO: 22) | |
| MERS-CoV | MLPFVQERIGLFIVNFFIFTVVCAITLLVCMAFLTATRLCVQCMTGFNTLLVQPALYLYNTGR |
| Envelope | SVYVKFQDSKPPLPPDEWV |
| protein (SEQ | |
| ID NO: 13) | |
| MERS-CoV | MSNMTQLTEAQIIAIIKDWNFAWSLIFLLITIVLQYGYPSRSMTVYVFKMFVLWLLWPSSMA |
| Membrane | LSIFSAVYPIDLASQIISGIVAAVSAMMWISYFVQSIRLFMRTGSWWSFNPETNCLLNVPFGG |
| protein (SEQ | TTVVRPLVEDSTSVTAVVTNGHLKMAGMHFGACDYDRLPNEVTVAKPNVLIALKMVKRQS |
| ID NO: 23) | YGTNSGVAIYHRYKAGNYRSPPITADIELALLRA |
| MERS-CoV | MASPAAPRAVSFADNNDITNTNLSRGRGRNPKPRAAPNNTVSWYTGLTQHGKVPLTFPPG |
| Nucleocapsid | QGVPLNANSTPAQNAGYWRRQDRKINTGNGIKQLAPRWYFYYTGTGPEAALPFRAVKDGI |
| protein (SEQ | VWVHEDGATDAPSTFGTRNPNNDSAIVTQFAPGTKLPKNFHIEGTGGNSQSSSRASSLSRNS |
| ID NO: 24) | SRSSSQGSRSGNSTRGTSPGPSGIGAVGGDLLYLDLLNRLQALESGKVKQSQPKVITKKDAAA |
| | AKNKMRHKRTSTKSFNMVQAFGLRGPGDLQGNFGDLQLNKLGTEDPRWPQIAELAPTASA |
| | FMGMSQFKLTHQNNDDHGNPVYFLRYSGAIKLDPKNPNYNKWLELLEQNIDAYKTFPKKE |
| | KKQKAPKEESTDQMSEPPKEQRVQGSITQRTRTRPSVQPGPMIDVNTD |

In some embodiments, the antigen is a bacterial antigen. Exemplary bacteria include, e.g., Helicobacter pylori, Borrelia burgdorferi, Legionella pneumophilia, Mycobacteria (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, Campylobacter sp., Enterococcus sp., Chlamydia sp., Haemophilus influenzae, Bacillus anthracia, Corynebacterium diphtheriae, Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidum, Treponema pertenue, Leptospira, Actinomyces israelii and Francisella tularensis.

In some embodiments, the antigen is a fungal antigen. Exemplary fungi include, e.g., Aspergillus, such as Aspergillus flavus, Aspergillus fumigatus, Aspergillus niger, Blastomyces, such as Blastomyces dermatitidis, Candida, such as Candida albicans, Candida glabrata, Candida guilliermondii, Candida krusei, Candida parapsilosis, Candida tropicalis, Coccidioides, such as Coccidioides immitis, Cryptococcus, such as Cryptococcus neoformans, Epidermophyton, Fusarium, Histoplasma, such as Histoplasma capsulatum, Malassezia, such as Malassezia furfur, Microsporum, Mucor, Paracoccidioides, such as Paracoccidioides brasiliensis, Penicillium, such as Penicillium marneffei, Pichia, such as Pichia anomala, Pichia guilliermondii, Pneumocystis, such as Pneumocystis carinii, Pseudallescheria, such as Pseudallescheria boydii, Rhizopus, such as Rhizopus oryzae, Rhodotorula, such as Rhodotorula rubra, Scedosporium, such as Scedosporium apiospermum, Schizophyllum, such as Schizophyllum commune, Sporothrix, such as Sporothrix schenckii, Trichophyton, such as Trichophyton mentagrophytes, Trichophyton rubrum, Trichophyton verrucosum, Trichophyton violaceum, Trichosporon, such as Trichosporon asahii, Trichosporon cutaneum, Trichosporon inkin, and Trichosporon mucoides.

In some embodiments, the antigen is from a parasite. Exemplary parasites include, e.g., parasites of the genus Plasmodium (e.g. Plasmodium falciparum, P. vivax, P. ovale and P. malariae), Trypanosoma, Toxoplasma (e.g., Toxoplasma gondii), Leishmania (e.g., Leishmania major), Schistosoma, or Cryptosporidium. In some embodiments the parasite is a protozoan. In some embodiments the parasite belongs to the phylum Apicomplexa. In some embodiments the parasite resides extracellularly during at least part of its life cycle. Examples include nematodes, trematodes (flukes), and cestodes. In some embodiments antigens from Ascaris or Trichuris are contemplated. In various embodiments, the antigen can originate from any component of the parasite. In some embodiments the antigen can be derived from parasites at any stage of their life cycle of the parasite, e.g., any stage that occurs within an infected organism such as a mammalian or avian organism. In some embodiments the antigen is derived from eggs of the parasite or substances secreted by the parasite.

In some embodiments, the antigen is a tumor antigen. In general, a tumor antigen can be any antigenic substance produced by tumor cells (e.g., tumorigenic cells or in some embodiments tumor stromal cells, e.g., tumor-associated cells such as cancer-associated fibroblasts). In some embodiments, a tumor antigen is a molecule (or portion thereof) that is differentially expressed by tumor cells as compared with non-tumor cells. Tumor antigens may include, e.g., proteins that are normally produced in very small quantities and are expressed in larger quantities by tumor cells, proteins that are normally produced only in certain stages of development, proteins whose structure (e.g., sequence or post-translational modification(s)) is modified due to mutation in tumor cells, or normal proteins that are (under normal conditions) sequestered from the immune system. Tumor antigens may be useful in, e.g., identifying or detecting tumor cells (e.g., for purposes of diagnosis and/or for purposes of monitoring subjects who have received treatment for a tumor, e.g., to test for recurrence) and/or for purposes of targeting various agents (e.g., therapeutic agents) to tumor cells. For example, in some embodiments, a chimeric antibody is provided, comprising an antibody of antibody fragment that binds a tumor antigen, and conjugated via click chemistry to a therapeutic agent, for example, a cytotoxic agent. In some embodiments, a tumor antigen is an expression product of a mutated gene, e.g., an oncogene or mutated tumor suppressor gene, an overexpressed or aberrantly expressed cellular protein, an antigen encoded by an oncogenic virus (e.g., HBV; HCV; herpesvirus family members such as EBV, KSV; papilloma virus, etc.), or an oncofetal antigen. Oncofetal antigens are normally produced in the early stages of embryonic development and largely or completely disappear by the time the immune system is fully developed. Examples are alphafetoprotein (AFP, found, e.g., in germ cell tumors and hepatocellular carcinoma) and carcinoembryonic antigen (CEA, found, e.g., in bowel cancers and occasionally lung or breast cancer). Tyrosinase is an example of a protein normally produced in very low quantities but whose production is greatly increased in certain tumor cells (e.g., melanoma cells). Other exemplary tumor antigens include, e.g., CA-125 (found, e.g., in ovarian cancer); MUC-1 (found, e.g., in breast cancer); epithelial tumor antigen (found, e.g., in breast cancer); melanoma-associated antigen (MAGE; found, e.g., in malignant melanoma); prostatic acid phosphatase (PAP, found in prostate cancer). In some embodiments, a tumor antigen is at least in part exposed at the cell surface of tumor cells. In some embodiments, a tumor antigen comprises an abnormally modified polypeptide or lipid, e.g., an aberrantly modified cell surface glycolipid or glycoprotein. It will be appreciated that a tumor antigen may be expressed by a subset of tumors of a particular type and/or by a subset of cells in a tumor.

In some embodiments, the tumor antigen is selected from the group consisting of: MAGE family members, NY-ESO-1, tyrosinase, Melan-A/MART-1, prostate cancer antigen, Her-2/neu, Survivin, Telomerase, WTi, CEA, gp100, Pmel17, mammaglobin-A, NY-BR-1, ERBB2, OA1, PAP, RAB38/NY-MEL-1, TRP-1/gp75, TRP-2, CD33, BAGE-1, D393-CD20n, cyclin-A1, GAGE-1, GAGE-2, GAGE-8, GnTVf, HERV-K-MEL, KK-LC-1, KM-HN-1, LAGE-1, LY6K, MAGE-A1, MAGE-A2, MAGE-A3, MAGE-A4, MAGE-A6, MAGE-A9, MAGE-A10, MAGE-A12, MAGE-C1, MAGE-C2, mucin K, NA88-A, SAGE, sp17, SSX-2, SSX-4, surviving, TAG-1, TAG-3, TRAG-3, XAGE-1b, BCR-AB1, adipophiln, AIM-2, ALDH1A1, BCLX(L), BING-4, CALCA, CD45, CD274, CPSF, cyclin D1, DKK1, ENAH, EpCAM, EphA3, EZH2, FGF5, glypican-3, G250, HER-2, HLA-DOB, hepsin, IDO1, IGF2B3, IL12Ralpha2, intestinal carboyxyl esterase, alpha-foetoprotein, kallikrein 4, KIF20A, Lengsin, M-CSF, M-CSP, mdm-2, Meloe, midkine, MMP-2, MMP-7, MUC1, MUC5AC, p53, PAX5, PBF, PRAME, PSMA, RAGE-1, RGS5, RhoC, RNF43, RU2AS, secerine 1, SOX10, STEAP1, telomerase, TPBG, mesothelin, Axl, and VEGF.

In some embodiments, the antigen is a whole cell, a whole parasite, a whole virus, a whole bacterium, or a whole nanoparticle, exosome, or microparticle comprising one or more antigens. In one example, a VHH may be conjugated to a beta islet cell and delivered under non-inflammatory conditions in order to induce beta islet cell tolerance in the course of organ or tissue replacement therapy. In yet another example, a VHH may be conjugated to a parasite and delivered under inflammatory conditions in order to induce a strong immune response against multiple parasite antigens at once.

### Anti-Inflammatory Agents and Proinflammatory Agents

As shown herein, the conjugates comprising VHH conjugated to an antigen to which immune tolerance is needed (e.g., a self-antigen), when administered to a subject under non-inflammatory conditions is more effective in inducing antigen-specific immune tolerance to the self-antigen. In some embodiments, the non-inflammatory condition is provided by attaching an anti-inflammatory agent to the same conjugate comprising the VHH and the antigen. In some embodiments, the non-inflammatory condition is provided by co-administering a VHH conjugated to an anti-inflammatory agent in addition to the VHH conjugated to a self-antigen.

An “anti-inflammatory agent” refers to a substance that reduces inflammation in the body. Anti-inflammatory agents block certain substances in the body that cause inflammation. Any anti-inflammatory agents known in the art can be used in accordance with the present disclosure. In some embodiments, the anti-inflammatory agent is a steroidal anti-inflammatory agent. In some embodiments, the steroidal anti-inflammatory agent is selected from the group consisting of: dexamethasone, prednisone, prednisolone, triamcinolone, methylprednisolone, and bethamethasone. In some embodiments, the anti-inflammatory agent is a nonsteroidal anti-inflammatory agent. In some embodiments, the nonsteroidal anti-inflammatory agent is selected from the group consisting of: aspirin, celecoxib, diclofenac, ibuprofen, ketoprofen, naproxen, oxaprozin, piroxicam, cyclosporin A, and calcitriol. In some embodiments, the anti-inflammatory agent used in accordance with the present disclosure is dexamethasone.

In some embodiments, the anti-inflammatory agent is an anti-inflammatory cytokine. An “anti-inflammatory cytokine” refers to a cytokine that inhibits the synthesis of IL-1, tumor necrosis factor (TNF), and other major proinflammatory cytokines and reduces inflammatory response. In some embodiments, the anti-inflammatory cytokine is selected from the group consisting of IL-10, IL-35, IL-4, IL-11, IL-13, and TGFβ.

The present disclosure, in other aspects, provides that the conjugates comprising VHH conjugated to an antigen to which immune response is needed (e.g., an antigen from a pathogen or a tumor antigen), when administered to a subject under inflammatory conditions is more effective in inducing antigen-specific immune response to the antigen. In some embodiments, the inflammatory condition is provided by attaching a proinflammatory agent to the same conjugate comprising the VHH and the antigen. In some embodiments, the non-inflammatory condition is provided by co-administering a VHH conjugated to a proinflammatory agent in addition to the VHH conjugated to an antigen. In some embodiments, the proinflammatory agent is selected from the group consisting of: TLR9 agonist (e.g., CpG ODN), LPS, HMGB1 proteins, IL2, IL12, and CD40L. In some embodiments, the pro-inflammatory agent is IL2.

### Methods of Treatment

Some aspects of the present disclosure provide methods of comprising administering to a subject in need thereof: (i) a conjugate comprising a VHH conjugated to an antigen to which immune tolerance is needed (e.g., a self-antigen) and an anti-inflammatory agent, wherein the VHH binds to a surface protein on an APC (e.g., MHCII or CD11c) or a (ii) a first conjugate comprising a VHH conjugated to an antigen to which immune tolerance is needed (e.g., a self-antigen) and a second conjugate comprising a second VHH conjugated to an anti-inflammatory agent, wherein the first VHH and the second VHH bind to one or more (e.g., same or different) surface proteins on an APC. In some embodiments, when two VHHs are administered, they are administered in the same composition, or in different compositions (e.g., sequentially). In some embodiments, the method is for inducing an immune tolerance to an antigen. In some embodiments, the method is for treating an autoimmune disease.

An “autoimmune disease” to a disorder that causes abnormally over activity of the immune system, which attacks and damages its own tissues. Non-limiting examples of autoimmune diseases include: rheumatoid arthritis (RA), systemic lupus erythematosus (SLE), Myasthenia Gravis (MG), Graves' Disease, Idiopathic Thrombocytopenia Purpura (ITP), Guillain-Barre Syndrome, autoimmune myocarditis, Membrane Glomerulonephritis, Type I or Type II diabetes, juvenile onset diabetes, multiple sclerosis, Reynaud's syndrome, autoimmune thyroiditis, gastritis, Celiac Disease, Vitiligo, Hepatitis, primary biliary cirrhosis, inflammatory bowel disease, spondyloarthropathies, experimental autoimmune encephalomyelitis, immune neutropenia, and immune responses associated with delayed hypersensitivity mediated by cytokines, T-lymphocytes typically found in tuberculosis, sarcoidosis, and polymyositis, polyarteritis, cutaneous vasculitis, pemphigus (e.g., Pemphigus vulgaris, Pemphigus foliaceus or Paraneoplastic pemphigus), pemphigold, Goodpasture's syndrome, Kawasaki's disease, systemic sclerosis, anti-phospholipid syndrome, and Sjogren's syndrome. In some embodiments, the autoimmune disease is selected from the group consisting of: multiple sclerosis, type II diabetes, Pemphigus vulgaris, myasthenia gravis, lupus, celiac diseases, and inflammatory bowel disease (IBD). In some embodiments, the autoimmune disease is selected from the group consisting of: autoimmune encephalomyelitis, acute disseminated encephalomyelitis (ADEM), optic neuritis (ON), transverse myelitis and brainstem encephalitis, rheumatoid arthritis, vasculitides, inflammatory bowel disease, multiple sclerosis, chronic obstructive pulmonary diseases, kidney disorders, posttransplantation fibrosis and in several types of cancer, autoimmune demyelinating disease, insulin autoimmune syndrome, type B insulin resistance syndrome, autoimmune gastritis, autoimmune diseases of the central nervous system, neurological autoimmune diseases, Type 1 diabetes, autoimmune thyroid disease, pernicious anemia, skin autoimmune disease, myasthenia gravis, neuromuscular junction autoimmune diseases. Different self-antigens may be used in the conjugates for treating different autoimmune diseases. One skilled in the art is able to identify the appropriate self-antigen to use.

Other aspects of the present disclosure provide methods of comprising administering to a subject in need thereof: (i) a conjugate comprising a VHH conjugated to an antigen to which immune response is needed (e.g., an antigen from a pathogen or a tumor antigen) and a proinflammatory agent, wherein the VHH binds to a surface protein on an APC (e.g., MHCII or CD11c) or a (ii) a first conjugate comprising a VHH conjugated to an antigen to which immune response is needed (e.g., an antigen from a pathogen or a tumor antigen) and a second conjugate comprising a second VHH conjugated to a proinflammatory agent, wherein the first VHH and the second VHH bind to one or more (e.g., same or different) surface proteins on an APC. In some embodiments, when two VHHs are administered, they are administered in the same composition, or in different compositions (e.g., sequentially). In some embodiments, the method is for inducing an immune response to an antigen. In some embodiments, the method is for treating infection caused by a pathogen (e.g., a microbial pathogen such as the ones described herein). In some embodiments, the methods is for treating cancer.

The cancer may be a primary or metastatic cancer. Cancers include, but are not limited to, adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, biliary tract cancer, osteosarcoma, fibrous histiocytoma, brain cancer, brain stem glioma, cerebellar astrocytoma, malignant glioma, glioblastoma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, acute lymphocytic and myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, extracranial germ cell tumor, extragonadal germ cell tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinema, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, choriocarcinoma, hematological neoplasm, adult T-cell leukemia, lymphoma, lymphocytic lymphoma, stromal tumors and germ cell tumors, or Wilms tumor. In some embodiments, the cancer is lung cancer, breast cancer, prostate cancer, colorectal cancer, gastric cancer, liver cancer, pancreatic cancer, brain and central nervous system cancer, skin cancer, ovarian cancer, leukemia, endometrial cancer, bone, cartilage and soft tissue sarcoma, lymphoma, neuroblastoma, nephroblastoma, retinoblastoma, or gonadal germ cell tumor.

In its broadest sense, the terms “treatment” or “to treat” refer to both therapeutic and prophylactic treatments. If the subject in need of treatment has a disease (e.g., autoimmune disease, infection, or cancer), then “treating the condition” refers to ameliorating, reducing or eliminating one or more symptoms associated with the disease or the severity of disease or preventing any further progression of disease. If the subject in need of treatment is one who is at risk of having a disease (e.g., infection or cancer), then treating the subject refers to reducing the risk of the subject having an infection cancer or preventing the subject from developing an infection or cancer.

A subject shall mean a human or vertebrate animal or mammal including but not limited to a rodent, e.g., a rat or a mouse, dog, cat, horse, cow, pig, sheep, goat, turkey, chicken, and primate, e.g., monkey. The methods of the present disclosure are useful for treating a subject in need thereof.

In some embodiments, the compositions described herein are pharmaceutical compositions. Pharmaceutically compositions that may be used in accordance with the present disclosure may be directly administered to the subject or may be administered to a subject in need thereof in a therapeutically effective amount. The term “therapeutically effective amount” refers to the amount necessary or sufficient to realize a desired biologic effect. For example, a therapeutically effective amount of a composition associated with the present disclosure may be that amount sufficient to ameliorate one or more symptoms of a targeted disease (e.g., autoimmune disease, infection, or cancer). Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular pharmaceutically compositions being administered the size of the subject, or the severity of the disease or condition. One of ordinary skill in the art can empirically determine the effective amount of a particular therapeutic compound associated with the present disclosure without necessitating undue experimentation.

Subject doses of the compositions described herein for delivery typically range from about 0.1 μg to 10 mg per administration, which depending on the application could be given daily, weekly, or monthly and any other amount of time there between. In some embodiments a single dose is administered during the critical consolidation or reconsolidation period. The doses for these purposes may range from about 10 μg to 5 mg per administration, and most typically from about 100 μg to 1 mg, with 2-4 administrations being spaced, for example, days or weeks apart, or more. In some embodiments, however, parenteral doses for these purposes may be used in a range of 5 to 10,000 times higher than the typical doses described above.

In some embodiments, a composition the present disclosure is administered at a dosage of between about 1 and 10 mg/kg of body weight of the mammal. In other embodiments composition of the present disclosure is administered at a dosage of between about 0.001 and 1 mg/kg of body weight of the mammal. In yet other embodiments, the composition of the present disclosure is administered at a dosage of between about 10-100 ng/kg, 100-500 ng/kg, 500 ng/kg-1 mg/kg, or 1-5 mg/kg of body weight of the mammal, or any individual dosage therein.

The compositions of the present disclosure are administered in pharmaceutically acceptable solutions, which may routinely contain pharmaceutically acceptable concentrations of salt, buffering agents, preservatives, compatible carriers, and optionally other therapeutic ingredients.

For use in therapy, an effective amount of the composition associated with the present disclosure can be administered to a subject by any mode that delivers the therapeutic agent or compound to the desired surface, e.g., mucosal, injection to cancer, systemic, etc. Administering the pharmaceutical composition of the present disclosure may be accomplished by any means known to the skilled artisan. Suitable routes of administration include but are not limited to oral, parenteral, intravenous, intramuscular, intranasal, sublingual, intratracheal, inhalation, ocular, vaginal, rectal and intracerebroventricular. In some embodiments, the composition is administered intravenously (e.g., via injection or infusion).

The pharmaceutical compositions of the present disclosure, when desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compositions may also be formulated as a depot preparation. Such long acting formulations may be formulated with suitable polymeric or hydrophobic materials (for example as an emulsion in an acceptable oil) or ion exchange resins, or as sparingly soluble derivatives, for example, as a sparingly soluble salt.

The compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The compositions of the present disclosure and optionally other therapeutics may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the present disclosure contain an effective amount of a therapeutic compound of the present disclosure optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present disclosure, and with each other, in a manner such that there is no interaction which would substantially impair the desired pharmaceutical efficiency.

The pharmaceutical compositions of the present disclosure may be delivered with other therapeutics for treating a disease (e.g., an autoimmune disease, infection, or cancer).

### EXAMPLES

### Example 1. Engineering the Modularity of Single Domain Antibody Targeting MHC Class II to Protect Against Autoimmune Diseases

Autoimmunity results from the recognition of self-antigens by components of the adaptive immune system. This explains the link of most autoimmune diseases with particular allelic variants of Class II MHC products that present the offending self-antigen. To treat autoimmune disease, induction of antigen-specific tolerance would be a highly desirable goal. Regardless of pathology, antigen presenting cells (APCs) are essential in the induction of disease, and conversely, APCs can be tolerogenic if they encounter antigen under non-inflammatory conditions. Described herein are nanobodies that recognize Class II MHC products, present on all APCs, which can be enzymatically conjugated to self-antigens such as a myelin oligodendrocyte glycoprotein (MOG) fragment in a preclinical model of autoimmune disease, experimental autoimmune encephalitis (EAE). Administration of these adducts under non-inflammatory conditions provides long-lasting protection against EAE. Similar adducts prevented hyperglycemia in a mouse model of accelerated type I diabetes and rheumatoid arthritis. Self-antigens were conjugated not only to nanobodies, but also a dexamethasone derivative, attached via a cleavable hydrazone linker. Co-administration of a Class II-MHC specific nanobody carrying the MOG peptide with that same nanobody, modified with the cleavable dexamethasone derivative, halted progression of disease in animals that were already symptomatic. While the precise target cell population responsible for therapeutic efficacy remains to be identified beyond the fact that these must be Class II MHC-positive cells, there is practical utility of these findings. The application of such antibody-drug conjugates in various inflammatory conditions should be considered as a viable treatment option.

### Introduction

Approximately 10% of the human population suffers from an auto-immune condition, accompanied by mild to life-threatening symptoms. In only select cases is there a plausible explanation for how disease is initiated. Immunotherapy of cancer using checkpoint blockade, while highly successful for a select set of malignancies, entails the risk of provoking autoimmunity by releasing the breaks on immune homeostasis. It is a clear example of an experimental trigger that uncovers the presence of harmful self-reactive cells, held in check until checkpoint blockade is applied.

Current treatments for autoimmune diseases include general immunosuppression, which blunts responses across the entire spectrum of antigens. This exposes patients to an increased risk of infection and possibly even malignancies. Because autoimmune diseases are often organ-specific, the immune component includes antigen-specific elements, either as triggers, as targets, or some combination of the two. This is perhaps best illustrated by various preclinical models of autoimmunity, where pathology can be elicited by administration of a defined antigen under the appropriate stimulatory conditions. For certain human autoimmune diseases, the antigens that induce pathology and recognized in the course of an autoimmune response are known. Examples include islet antigens in the case of type-1 diabetes, components of the myelin sheath in multiple sclerosis, and citrullinated antigens in the case of arthritis.

Nanoparticles composed of peptide loaded MHC products have been used to elicit a form of both Class I and Class II MHC-restricted tolerance. A further striking example is the ability of red blood cells, modified with a self-antigen, to induce a profound state of antigen non-responsiveness. This trait has been attributed to the exceptional turnover rate in comparison with other cell types, and the need to eliminate red blood cell remnants without causing an inflammatory response. The phenomenon of tolerogenic elimination of cell remnants is not limited to red blood cells, as transfusion of chemically modified, apoptotic peripheral blood lymphocytes can also dampen auto-immune responses.

Reported herein is the development and characterization of alpaca-derived single domain antibody fragments (nanobodies/VHHs) that recognize Class II MHC molecules. These nanobodies target all Class II MHC-positive cells, including antigen presenting cells (APCs). At one-tenth the size of conventional immunoglobulins, the small size of nanobodies ensures excellent tissue penetration and rapid clearance from the circulation. This makes VHHs ideal vehicles for targeted delivery of payloads of interest, such as antigenic peptides or small molecule drugs. Furthermore, an engineering strategy that used sortase A, a S. aureus-derived transpeptidase was established. It enabled site-specific modification of these VHHs at their C-terminus. Sortase-modified Class II MHC-specific nanobodies were used as imaging agents for positron emission tomography, the results of which were consistent with a short circulatory half-life, paired with excellent targeting properties. These methods likewise allowed the installation of a wide range of antigens involved in infectious and autoimmune disease. There is broad consensus that engagement of antigen presenting cells under non-inflammatory conditions can lead to tolerance, whereas administration under inflammatory conditions, for example in the presence of adjuvants, can elicit a strong protective response against foreign antigens. Valency, aggregation state and dose of the antigen are additional parameters that can make the pendulum swing from tolerogen to immunogen. The distribution of a diverse set of APCs over different anatomical sites and its dynamics pose a challenge for the identification of the relevant tolerogenic APC in vivo. The purpose of this study was not so much to pin down a particular APC (sub)set responsible for tolerance induction, but rather to demonstrate the efficacy of using VHHs to target the Class II MHC-positive cell population in different settings, including the targeted delivery of dexamethasone, an immunosuppressive small molecule. The clinical use of purified dendritic cells loaded with self-peptides is a matter of record, but there would be a practical advantage to avoiding cell-based therapies if a purely proteinaceous preparation could be administered to the same effect. Indeed, findings indicated that the combination of a Class II MHC VHH-peptide adduct, in combination with the same VHH conjugated to dexamethasone was remarkably effective at arresting progression of EAE in animals with overt signs of disease.

### Methods

### Expression of VHHs and Endotoxin Removal

WK6 E. coli containing the plasmid encoding corresponding VHHs were grown to mid-log phase at 37° C. in Terrific Broth plus ampicillin and induced with 1 mM IPTG overnight at 30° C. Bacteria were harvested by centrifugation at 5,000×g for 15 minutes at 4° C. and then resuspended in 25 mL 1×TES buffer (200 mM Tris, pH 8, 0.65 mM EDTA, 0.5 M sucrose) per liter culture and incubated for 1 hour at 4° C. with agitation. Resuspended cells were then subjected to osmotic shock by 1:4 dilution in 0.25×TES buffer and incubation overnight at 4° C. The periplasmic fraction was isolated by centrifugation at 5,000×g for 30 minutes at 4° C. and then loaded onto Ni-NTA (Qiagen) in 50 mM Tris, pH 8, 150 mM NaCl, and 10 mM imidazole. Protein was eluted in 50 mM Tris, pH 8, 150 mM NaCl, 500 mM imidazole, and 10% glycerol and then loaded onto a Superdex 75 10/300 column in 50 mM Tris, pH 8, 150 mM NaCl, 10% glycerol. The peak fractions were recovered and rebounded to Ni-NTA to be depleted of LPS (<2 IU/mg). Bound VHHs were washed with 40 column volumes of PBS+0.1% TritonX-114 and eluted in 2.5 column volumes endotoxin-free PBS (Teknova) with 500 mM imidazole. Imidazole was removed by PD10 column (GE Healthcare), eluting in LPS-free PBS. Recombinant VHH purity was assessed by SDS/PAGE and LC-MS.

### Chemical Synthesis of GGG-Antigens, GGG-Cy5, and GGG-DEX

The peptides were synthesized on 2-chlorotrityl resin (ChemImpex) following standard solid phase peptide synthesis (SPPS) protocol or ordered on GenScript. For GGG-Cy5, GGGC (SEQ ID NO: 61) (7.0 mg, 24 μmol) was dissolved in DMSO (Sigma Aldrich) (400 μL) and was added to Cyanine 5 maleimide (Lumiprobe) (5.0 mg, 7.8 μmol). The resulting mixture was gently agitated at room temperature until LC-MS analysis show no remaining starting material. The ligated product was then purified by RP-HPLC and lyophilized. LC-MS calculated for GGG-Cy5: C₄₇H₆₂N₈O₈S₂[M+H]+ was 898.44, found 898.56. The resulting powder was stored at 4° C.

For GGG-Dexamethasone (DEX), in the first reaction, dexamethasone (Sigma Aldrich) (25 mg, 64 μmol) and N-β-maleimidopropionic acid hydrazide (ThermoFisher) (40 mg, 135 μmol) was dissolved in 3.0 mL of dry MeOH (Sigma Aldrich) and one drop of TFA was added to the solution. The resulting mixture was agitated overnight at room temperature. The MeOH was then evaporated, the precipitate dissolved in DMSO (1.0 mL), purified by RP-HPLC and lyophilized. LC-MS calculated for DEX-maleimide: C₂₉H₃₇FN₃O₇ [M+H]+ was 558.26, found 558.32. The resulting powder was stored at −20° C. In a second reaction, DEX-maleimide (20 mg, 36 μmol) and GGGC (SEQ ID NO: 61) (21 mg, 72 μmol) were dissolved in 5% 0.1 M NaHCO₃ in DMSO (1.0 mL). The resulting mixture was agitated at room temperature until completion of the reaction. Once no starting material was left, the reaction was directly purified by RP-HPLC and lyophilized. LC-MS calculated for GGG-DEX: C₃₈H₅₃FN₇O₁₂S [M+H]+ was 850.35, found 850.21. The resulting peptide was stored at −20° C. and re-dissolved in PBS before at the right concentration before sortase ligation.

C-Terminal Sortagging (Using LPETGG (SEQ ID NO: 43) of VHH or GFP with GGG-Carrying Moieties

Sortagging reactions were carried out in 1 mL mixture containing Tris HCl (50 mM, pH 7.5), CaCl₂) (10 mM), NaCl (150 mM), triglycine-containing probe (500 μM), GGG-containing probe (100 μM), and 5M-Sortase A (5 μM). After incubation at 4° C. with agitation for 1.5 hours, unreacted VHH and 5M-SrtA were removed by adsorption onto Ni-NTA agarose beads. The unbound fraction was concentrated and excess nucleophile with an Amicon 3,000 kDa MWCO filtration unit (Millipore). Reaction products were analyzed by LC-MS for purity and stored at −80° C.

### Mice

All animals were housed in the animal facility of Boston Children's Hospital (BCH) and were maintained according to protocols approved by the BCH Committee on Animal Care. C57BL/6J (CD45.2+), B6.SJL-Ptprc (CD45.1+), NOD/SCID, BALB/c, B6/2D2, NOD/BDC2.5, Balbc/DO11.10, CD11c-DTR, μMT−/−, Batf3−/−, LAG3−/−, and FoxP3-DTR mice were either purchased from the Jackson Laboratory or bred in house. MHCII-GFP and PD1−/− mice were bred in house. OTI Rag2−/− and HLA-DR4-IE-transgenic C57BL/6 IAb null mice were purchased from Taconic.

### Flow Cytometry Analysis

Cells were harvested from spleen, lymph nodes, or other organs and were dispersed into RPMI1640 through a 40-micron cell strainer using the back of a 1 mL syringe plunger. Cell mixture were subjected to hypotonic lysis (NH₄Cl) to remove red blood cells, washed twice in FACS buffer (2 mM EDTA and 1% FBS in PBS) and resuspended in FACS buffer containing the corresponding fluorescent dye-conjugated antibodies. All staining was carried out at 1:100 dilution and with Fc block for 30 minutes at 4° C. in dark. Samples were washed twice with FACS buffer before further analysis. All flow data were acquired on a FACS Fortessa flow cytometer (BD Biosciences) and analyzed using FlowJo software (Tree Star).

Antibodies used in this study are listed in Table 3.

TABLE 3
Antibodies used for immune phenotype characterization in data figures

| Target | Color | Clone | Manufacturer | Cat. Number |
|---|---|---|---|---|
| B220 | Alexa 700 | RA3-6B2 | eBioscience | 56-0452-82 |
| CD115(CSF1R) | PeCy7 | AFS98 | BioLegend | 135523 |
| CD11b | APC | M1/70 | BioLegend | 101212 |
| CD11b | BV711 | M1/70 | BioLegend | 101242 |
| CD11c | PerCP | N418 | BioLegend | 117326 |
| CD11c | BV605 | N418 | BioLegend | 117333 |
| CD19 | PE | 6D5 | BioLegend | 115508 |
| CD27 | APC-Cy7 | LG.3A10 | BioLegend | 124225 |
| CD3 | BV421 | 17A2 | BioLegend | 100228 |
| CD4 | FITC | GK1.5 | BioLegend | 100406 |
| CD4 | APC | RM4-5 | BioLegend | 100516 |
| CD4 | PeCy7 | RM4-5 | BioLegend | 100528 |
| CD44 | PE | IM7 | BioLegend | 103008 |
| CD45.1 | PeCy7 | A20 | eBioscience | 25-0453-82 |
| CD45.2 | APC | 104 | BioLegend | 109814 |
| CD45.2 | PeCy7 | 104 | BioLegend | 109830 |
| CD5 | PE | 53-7.3 | BioLegend | 100607 |
| CD62L | PeCy7 | MEL-14 | BioLegend | 104418 |
| CD8 | APC-Cy7 | 53-6.7 | BioLegend | 100714 |
| CD95 | PeCy7 | Jo2 | BD Bioscience | 557653 |
| Fc block (CD16/CD32) | N/A | 93 | BioLegend | 101302 |
| FoxP3 | eFluor 450 | FJK-16s | eBioscience | 2136519 |
| FoxP3 | FITC | FJK-16s | Invitrogen | 430671 |
| IFNγ | FITC | XMG1.2 | BioLegend | 505806 |
| IgD | BV711 | 11-26c.2a | BioLegend | 405731 |
| IL17a | PE | TC11-18H10.1 | BioLegend | 506904 |
| Lag3 (CD223) | PE | eBioC9B7W | eBioscience | 12-2231-81 |
| LAP | PE | TW7-16B4 | BD Bioscience | 563143 |
| MHC Class II (I-A/I-E) | PE | M5/114.15.2 | BioLegend | 107608 |
| PD1 (CD279) | PeCy7 | 29F.1A12 | BioLegend | 135216 |
| PDCA-1 (CD317) | PE | 129C1 | BioLegend | 127103 |
| TCRa3.2 | APC | RR3-16 | Invitrogen | 17-5799-82 |
| TCRb11 | PerCP-eFluor 710 | RR3-15 | eBioscience | 46-5827-80 |
| Tim-3 (CD366) | PE | RMT3-23 | BioLegend | 119704 |

### Experimental Autoimmune Encephalomyelis (EAE) Model in C57BL/6J Mice

Female C57BL/6 mice (10-12 weeks of age) or other mouse lines with C57BL/6J genetic background were immunized with Hooke kits: an emulsion of MOG₃₅₋₅₅ in CFA and PTX in PBS according to the manufacturer's instructions (Hooke laboratories). Mice were scored daily, starting on day 7 post-immunization by an investigator blinded to the experimental treatment of individual mice. Mice were assigned to different experimental treatments randomly and cohoused together to eliminate inter-cage variability. All treatments were carried out on at least 3 mice and in at least two independent experiments, as indicated in the figure legends. All animals were included in the analyses. Clinical score is defined as follows: 1, limp tail; 2, partial hind leg paralysis; 3, complete hind leg paralysis; 4, complete hind and partial front leg paralysis; and 5, moribund. Easy access to wet food and water was provided for the experimental mice throughout the disease progression. Unless indicated otherwise, for prophylactic treatment, 20 μg sortagged VHH-antigens were intravenously administered 7 days prior to induction of EAE. For therapeutic treatment, 20 μg VHH_{MHCII}-OVA₃₂₃₋₃₃₉, VHH_{MHCII}-MOG₃₅₋₅₅, or 20 μg VHH_{MHCII}-MOG₃₅₋₅₅ mixed 20 μg VHH_{MHCII}-DEX were administered on the day of EAE when the mice exhibited symptoms defined as clinical score of 1, 2, and 3 as indicated. At day 30 post-EAE induction or when mice reached clinical score of 4, mice were sacrificed by asphyxiation and then perfused with 5 mM EDTA in PBS. Spinal cords were isolated and fixed in 10% (wt/vol) formalin solution (Sigma), embedded in paraffin, sectioned at 20 μm, and stained with H&E or Luxol Fast Blue (Harvard Medical School Rodent Histology Core Facility). Stained sections were imaged at 4× and 10× magnification. Isolation of the immune cells that infiltrate the spinal cord was carried out by homogenizing the spinal cord, followed by 38% Percoll (Sigma) gradient separation (100% Percoll is 1.123 g/mL). Isolated cells were plated in 48-well plates and treated with 50 ng/mL PMA (Sigma) and 500 ng/mL ionomycin (Sigma) for 2 hours at 37° C. in complete RPMI media, followed by the addition of 10 μg/mL Monensin (Sigma) and incubated for 2 more hours. Cells were then surface stained, fixed, and permeabilized using Foxp3/Transcription Factor Staining Buffer Set (ThermoFisher Scientific, 00-5523-00) according to the manufacturer's protocol. Intracellular and Foxp3 staining were performed according to the manufacturer's protocols and cell samples were then used for flow cytometry.

For cytokine storm analysis, blood samples were taken 5 hours post therapeutic treatment with 20 μg VHH_{MHCII}-MOG₃₅₋₅₅, VHH_{MHCII}-OVA₃₂₃₋₃₃₉, or 20 μg VHH_{MHCII}-MOG₃₅₋₅₅+20 μg VHH_{MHCII}-DEX on the first day these EAE mice reached clinical score of 3. Blood was collected in EDTA containing tubes and plasma was isolated via repeated centrifugation (500 g, 5 min, 4° C.). Plasma was stored at −80° C. until further analysis of tumor necrosis factor alpha (TNF-α) and interleukin 6 (IL-6). TNF-α (ThermoFisher, 88-7324-22) and IL-6 (ThermoFisher, 88-7064-22) ELISAs were conducted according to manufacturer's protocol.

### Cellular Subset Depletion

CD8 T-cells were depleted by administering 400 μg of anti-CD8a depleting antibody (clone 2.43, BioXCell) intraperitoneally twice weekly beginning 2 weeks prior to prophylactic treatment with VHH-antigen and throughout the EAE observation window. Macrophage subsets were ablated by injecting 300 μg anti-CSF1R (clone AFS98, BioXCell) every other day from 2 weeks prior to prophylactic treatment up to the end of the experimental set up. To deplete DCs, 100 ng DTX (Sigma) was administered intraperitoneally into CD11c-DTR mice 2 days prior to VHH-antigen administration. For depleting Tregs, FoxP3-DTR mice were injected with 3 doses of 1 μg DTX (Sigma) intraperitoneally at day −9, −8, and −1 prior to prophylactic treatment with VHH-antigen and weekly afterwards until the end of observation window. Cellular depletions were confirmed by flow cytometry of PBMCs or splenocytes.

### 2D2 CD4 T-Cell Adoptive Transfer and Challenge

Splenic and iLNs-derived CD4 T cells from 2D2 mice were enriched by negative selection using magnetic beads (Miltenyi Biotec, 130-104-453) and labeled with Violet CellTrace (ThermoFisher Scientific, C34571) as per the manufacturer's protocol. 500,000 of these 2D2 CD4+ T cells were transferred into CD45.1+ mice. Transfusion of 20 μg VHH_{MHCII}-OVA_{323-339, 20} μg VHH_{MHCII}-MOG₃₅₋₅₅, equimolar of MOG₃₅₋₅₅ peptides, or 100 μg MOG₃₅₋₅₅ peptides mixed with 25 μg anti-CD40 (SouthernBiotech) and 50 μg PolyI:C (Sigma) as adjuvant was carried out the day after adoptive transfer. At day 3, 5, and 10, mice were sacrificed and spleens, iLNs, and blood were collected and analyzed by flow cytometry. Some 2D2 T cell adoptively transferred mice were also challenged on day 3 or 10 with 100 μg MOG₃₅₋₅₅ in CFA subcutaneously. Mice were sacrificed 7 or 5 days later as indicated in the respective experimental set up. Spleens, iLNs, and blood were harvested and analyzed by flow cytometry.

### 2D2 CD4 T-Cell RNA-Seq

Cells were sorted and lysed in RLT lysis buffer (Qiagen) supplemented with β-mercaptoethanol. RNA was the isolated using a RNeasy Micro kit (Qiagen) according to the manufacturer's protocol. 20 ng of RNA were used as input to a modified SMART-seq2 protocol. The resulting library was confirmed using a High Sensitivity DNA Chip run on a Bioanalyzer 2100 system (Agilent), followed by library preparation using the Nextera XT kit (Illumina) and custom index primers according to the manufacturer's protocol. Final libraries were quantified using a Qubit dsDNA HS Assay kit (Invitrogen) and a High Sensitivity DNA chip run on a Bioanalyzer 2100 system (Agilent). All libraries were sequenced using Nextseq High Output Cartridge kits and a Nextseq 500 sequencer (Illumina). Sequenced libraries were demultiplexed using the bcl2fastq program and the resulting Fastq data were trimmed and cropped with Trimmomatic. Alignment to the mouse mm10 reference genome and gene expression counts were carried out using Kallisto. Principal Component Analyses (PCA) were carried out in R. To test for differential gene expression from our RNA-seq data and differential chromatin accessibility in individual loci, the DEseq2 method was used. Volcano plots and heatmaps were generated in Python 3.6 using NumPy 1.12.1, and Matplotlib 2.2.2. For functional analyses, Gorilla (Gene Ontology Enrichment Analysis and Visualization Tool) was used to find enriched Gene Ontology (GO) terms in the up-regulated and down-regulated subsets of the top 500 most differentially expressed genes.

### Type 1 Diabetes (T1D) Model in NOD/SCID Mice

Spleen and inguinal lymph nodes were harvested from 7-9-week-old BDC2.5 mice. Cells were resuspended in complete RPMI (RPMI supplemented with 2 mM glutaMAX, 10 mM HEPES, non-essential amino acids, 1 mM sodium pyruvate, 55 μM β-mercaptoethanol, 10% heat-inactivated FBS) supplemented with 0.5 μM p31 peptide (BDC2.5 mimotope, GenScript) and plated in tissue culture dishes at 1 million cells/mL. After four days, cells were harvested, washed twice and resuspended in PBS. 5 million cells were adoptively transferred into 9-12-week-old female NOD.SCID mice via retro-orbital injection. Saline, 20 μg VHH_{MHCII}-p31, or VHH_{MHCII}-MOG₃₅₋₅₅ were infused into the mice a day or 5 days later as indicated. Blood glucose measurements were carried out every other day for 2 weeks and weekly for up to 1-2 months. Mice were considered diabetic when their blood glucose level exceeded 260 mg/dL for two subsequent weeks as measured by using the Active meter (Accu-Chek) (range 20-600 mg/dL) with corresponding Aviva Plus test strips (Accu-Check).

Mice were sacrificed via asphyxiation at the 2-month endpoint or when blood glucose levels exceeded 600 mg/dL for two subsequent weeks. The pancreas was fixed for further immunohistochemistry analysis, i.e. H&E staining (Harvard Medical School Rodent Histology Core Facility). In a separate cohort of mice, spleens, inguinal/pancreatic lymph nodes and pancreas were harvested at day 14 post adoptive transfer for flow cytometry analysis.

### Rheumatoid Arthritis (RA) Model in BALB/c Mice

Spleen and lymph nodes were collected from DO11.10 mice. CD4+ T cells from these mice were enriched by negative selection using magnetic beads (Miltenyi Biotec, 130-104-453). APCs were obtained by irradiating DO11.10 splenocytes at 2000 rad. Differentiation of these naïve CD4 T cells into Th1 phenotypes was induced by culturing them as follows: 200,000 CD4+ T cells and 2 million APCs were co-cultured in complete RPMI containing 0.3 μM OVA₃₂₃₋₃₃₉ (GenScript), 5 ng/mL IL12 (PeproTech), and 10 μg/mL anti-IL4 mAb (R&D Systems) for 3 days. Cells were then harvested, washed, and counted. A total of 2 million Th1 DO11.10 T cells were injected intravenously into BALB/c recipients. One day following T cell transfer, recipients were immunized subcutaneously with 100 μg OVA in CFA (Sigma-Aldrich). At day 11, heat aggregated OVA (HOA) was injected into the left paw of the mice and paw thickness was measured daily up to day 18. Mice were then sacrificed, and their paws were removed and fixed in 10% (wt/vol) formalin solution (Sigma), embedded in paraffin, sectioned at 20 μm, and stained with Toluidine Blue (Harvard Medical School Rodent Histology Core Facility). Stained sections were imaged at 4× and 10× magnification. Popliteal lymph nodes were also collected and cells were restimulated in vitro with 1 mg/mL OVA in complete RPMI for 3 days for IFN-γ production. IFNγ was measured using the Mouse IFN-γ ELISA Set (BD Biosciences, 555138) per manufacturer's protocol. Sera was also collected at D18 end point for ELISA assays to measure anti-OVA and anti-OVA₃₂₃₋₃₃₉ antibody responses. 96-well plates were coated with 10 μg/mL of OVA or GFP-OVA₃₂₃₋₃₃₉ (generated by sortagging GFP-LPETGG_{(SEQ ID NO: 43)} with GGG-OVA₃₂₃₋₃₃₉) proteins in PBS overnight at 4° C. and incubated in blocking buffer (0.05% Tween20+2% BSA in PBS) before addition of serum samples. Incubation with tested serum was for 3 hours at room temperature. Plates were washed four times with PBS, incubated with goat anti-mouse IgG-HRP (SouthernBiotech) at 1:10,000 in blocking buffer for 1 hour, and developed with 3,3′,5,5′-tetramethylbenzidine (TMB) liquid substrate reagent (Sigma). The reaction was stopped with 1 M HCl and absorbance was read at 450 nm.

### OTI CD8 T-Cell Adoptive Transfer and Challenge

Spleen and lymph nodes were collected from OTI Rag2^{-/-} mice. CD8+ T cells from OTI Rag2^{-/-} were enriched by negative selection using magnetic beads (Miltenyi Biotec, 130-095-236) and labeled with Violet CellTrace as the manufacturer's protocol. 500,000 CD8+ T cells were transferred intravenously into CD45.1+ mice. Transfusions of 20 μg VHH_{MHCII}-OTI or VHH_{MHCII}-ORF8 were carried out the day after adoptive transfer. Mice were challenged on day 10 with 25 μg OTI peptide in CFA (Sigma) and then sacrificed 5 day later for analyses. Spleens, iLNs, and blood were harvested and splenocytes were analyzed by flow cytometry.

Two million splenocytes were plated in 96-well round-bottomed plates and treated with Cell Stimulation Mixtures (eBioscience) and Brefeldin A (eBioscience) for 3 days at 37° C. in complete RPMI [RPMI 1640, 10% (vol/vol) heat-inactivated FBS, 50 μM β-mercaptoethanol, 100 U/mL Pen/Strep, 1× Gibco MEM Non-Essential Amino Acids Solution (Life Technologies), 1 mM Sodium pyruvate, 1 mM HEPES] supplemented with 1 mg/mL OVA peptides. Supernatant was collected and utilized for ELISA to measure Interferon gamma (IFNγ) production. IFNγ was measured using the Mouse IFN-γ ELISA Set (BD Biosciences, 555138) per manufacturer's protocol.

### Repeated Transfusions of VHH_{MHCII}-OB1

OB1 is a 17-mer B cell epitope derived from OVA. C57BL6/J recipient mice were intravenously injected with 20 μg VHH_{MHCII}-OB1, equimolar amount of OVA proteins, or PBS at day 0. Subsequent boosts were carried out on day 7 and day 14. Serum samples were collected pre-immunization and 7 days after the last boost. For OVA-specific and OB1 peptide-specific ELISA, 96-well plates were coated with 10 μg/mL of OVA or GFP-OB1 proteins in PBS overnight at 4° C. and incubated in blocking buffer (0.05% Tween20+2% BSA in PBS) before addition before addition of serum samples. Incubation with tested serum was for 3 hours at room temperature. Plates were washed four times with PBS, incubated with goat anti-mouse IgG-HRP (SouthernBiotech) at 1:10,000 in blocking buffer for 1 hour, and developed with 3,3′,5,5′-Tetramethylbenzidine (TMB) liquid substrate reagent (Sigma). The reaction was stopped with 1 M HCl and absorbance was read at 450 nm.

### EAE Model in HLA-DR4-IE-Transgenic C57BL/6 IAb Null Mice

DR4-IE mice were immunized with 400 μg of human PLP₁₇₅₋₁₉₂ (hPLP₁₇₅₋₁₉₂) emulsified in CFA subcutaneously. The mice also received 300 ng of Pertussis toxin intravenously on days 0 and 3. At day 7, mice were given second boost subcutaneously with 400 μg of hPLP₁₇₅₋₁₉₂ emulsified in Incomplete Freund's Adjuvant (IFA). Mice were weighed and scored daily starting on day 7 after immunization. The clinical score system was carried out similarly as the EAE model in C56BL/6J mice. On the first day a mouse reached a clinical score of 3, either 2 μg anti-human MHCII VHH (VHH_{hMHCII}) carrying an irrelevant peptide control or 20 μg VHH_{hMHCII}-hPLP₁₇₅₋₁₉₂ mixed with 20 μg VHH_{hMHCII}-DEX was administered intravenously. Flow cytometry of the spinal cords was described as above.

### Statistical Methods

All data represented at least two independent experiments. All statistical analyses were performed using Prism 6. Statistical methods used are indicated in the corresponding legend of each figure. Statistically significant differences are indicated by asterisks as follows: *p<0.05; **p<0.01; ***p<0.001.

### Results

A Single Dose of VHH_{MHCII}-MOG₃₅₋₅₅ Provides Durable Protection Against Induction of Experimental Autoimmune Encephalomyelitis (EAE).

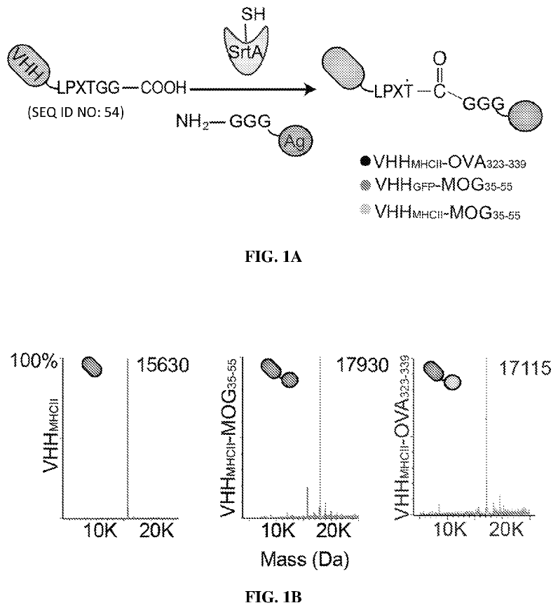

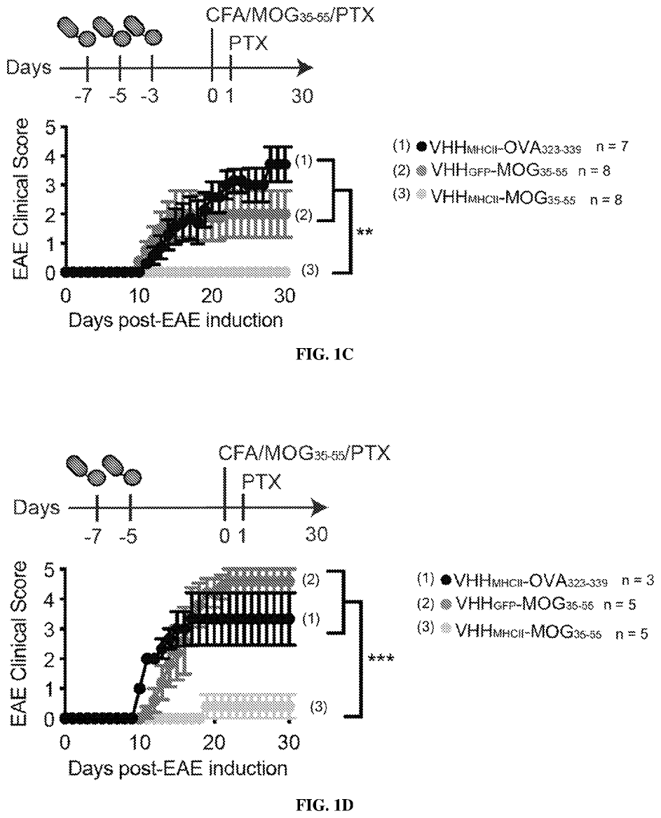

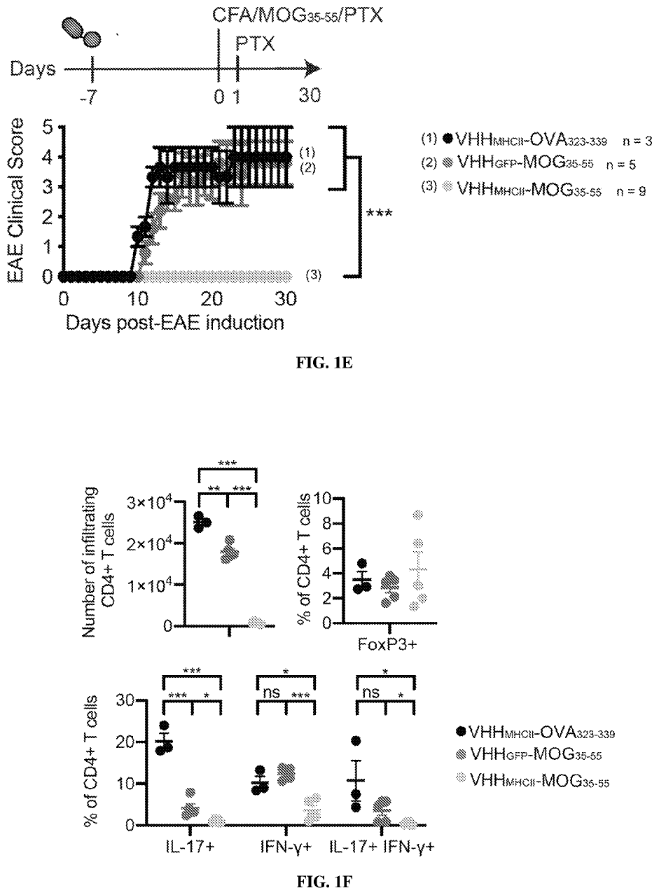

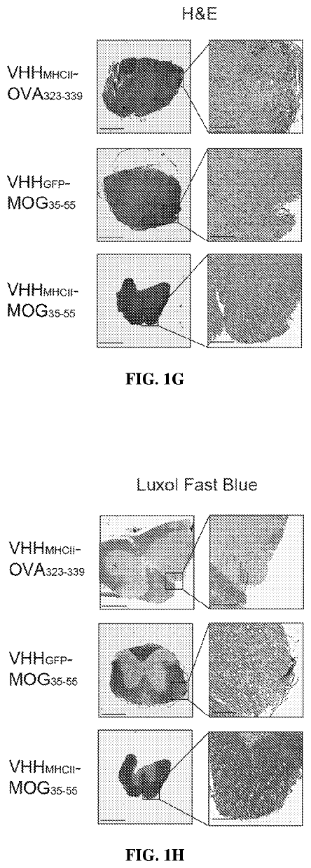

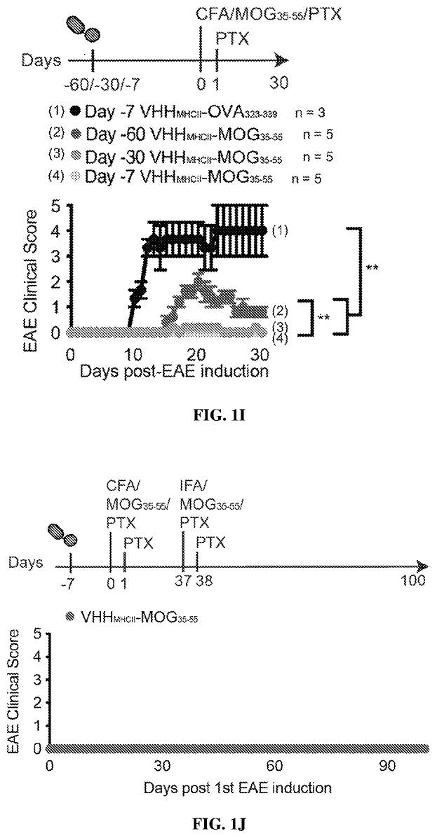

Described herein were the generation and characterization of an alpaca-derived single domain antibody (i.e. VHH_{MHCII}) that recognizes a wide range of mouse Class II MHC molecules, including I-A^{b} and I-A^{d}. This VHH was engineered to carry a sortase recognition motif—LPETGG (SEQ ID NO: 43)—to allow its site-specific ligation (FIG. 1A) to antigenic peptides and small molecules modified with at least one suitably exposed glycine residue(s). Antigenic peptides conjugated to VHH in this way are listed in Table 4. Purified VHH-peptide adducts were characterized by LC-MS (FIG. 1B and FIG. 8) to verify identity, homogeneity, and purity.

TABLE 4
Amino acid sequences of antigenic peptide probes

| | Amino Acid Sequence (N-terminus to |
|---|---|
| Peptide | C-terminus) |
| GGG-MOG₃₅₋₅₅ | GGGCKKGSMEVGWYRSPFSRVVHLYRNGK |
| | (SEQ ID NO: 4) |
| GGG-OT-I/GGG- | GGGCKKFRSIINFEKL (SEQ ID NO: 5) |
| OVA₂₅₇₋₂₆₄ | |
| GGG-OT-II/GGG- | GGGCKKISQAVHAAHAEINEAGR (SEQ ID |
| OVA₃₂₃₋₃₃₉ | NO: 6) |
| GGG-OB1 17-mer | GGGCKKFDKLPGFGDSIEAQGGK (SEQ ID |
| | NO: 7) |
| GGG-p31 | GGGCKKYVRPLWVRME (SEQ ID NO: 8) |
| GGG-ORF8₆₀₄₋₆₁₂ | GGGCKKFRKNYIFEEKL (SEQ ID NO: 9) |

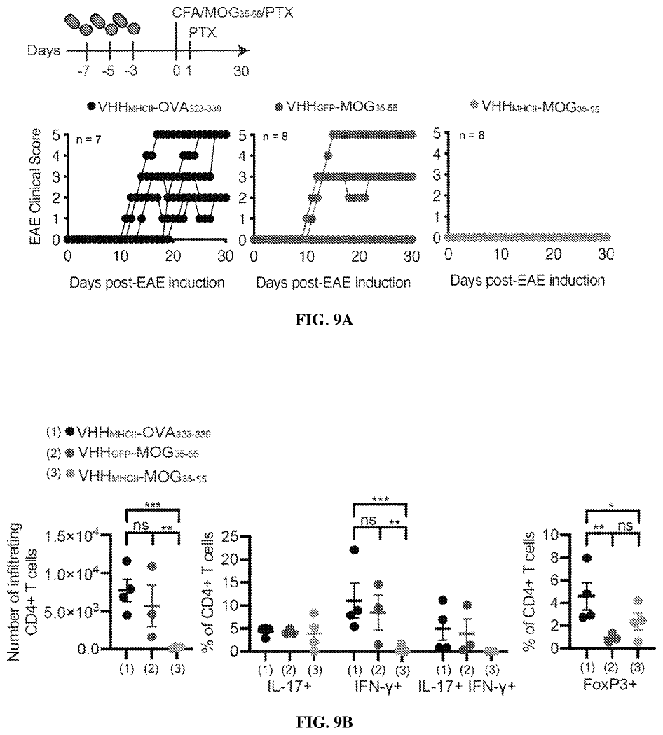

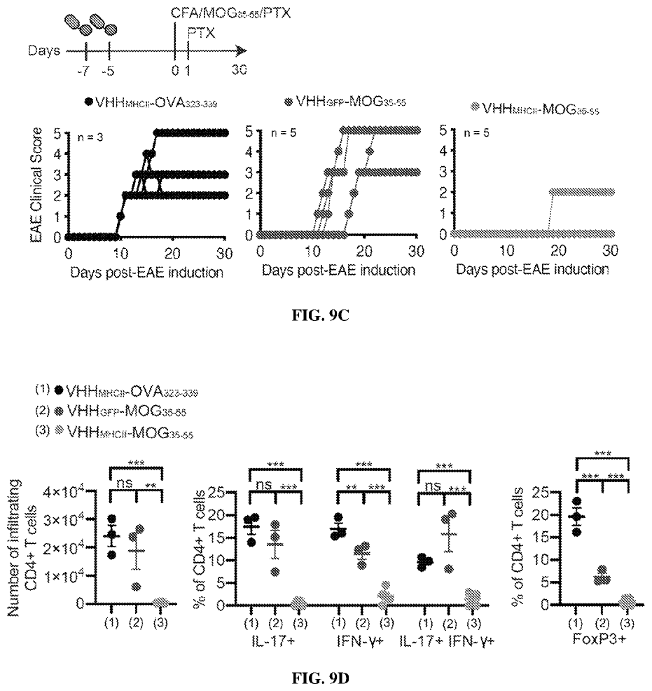

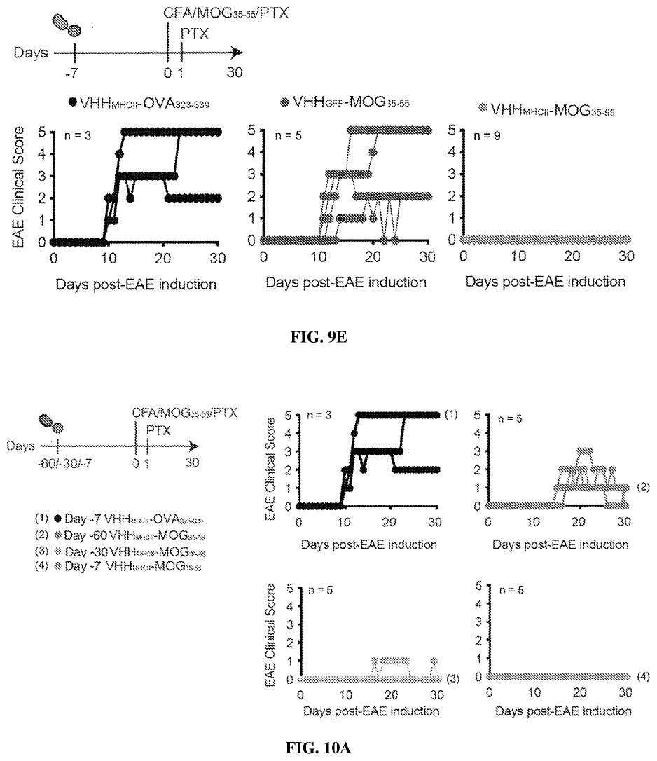

Immunization of C57BL6 mice with MOG₃₅₋₅₅ under inflammatory conditions, i.e. in the presence of complete Freund's adjuvant (CFA) and pertussis toxin (PTX), within 10-14 days elicits experimental autoimmune encephalitis (EAE), a multiple sclerosis-like condition. Prior administration of MOG₃₅₋₅₅, delivered to MHCII+ APCs under non-inflammatory conditions, was predicted to interfere with the induction of EAE. To determine a possible dose of VHH-peptide adduct that might interfere with onset and severity of symptoms, 3 doses of 20 μg VHH_{MHCII}-MOG₃₅₋₅₅ adduct were administered intravenously (i.v.) 7 days prior to induction of disease. This treatment completely suppressed induction of EAE, whereas mice that received the identical amount of VHH_{MHCII} conjugated to an irrelevant peptide (VHH_{MHCII}-OVA₃₂₃₋₃₃₉), or MOG₃₅₋₅₅ peptide linked to a VHH of irrelevant specificity (VHH_{GFP}) progressed to EAE (FIG. 1C). Even a single injection of 20 μg VHH_{MHCII}-MOG₃₅₋₅₅ achieved full protection (FIGS. 1D and 1E), therefore this dose was used in all subsequent experiments. Flow cytometry of CD4+ lymphocyte infiltrates recovered from the spinal cord of diseased mice at day 15-18 after immunization and of protected mice at day 30 after EAE induction were consistent with the observed disease scores: diseased mice showed a pronounced influx of IL17 and IFNγ-producing CD4+ T cells, as well as some Foxp3⁺ CD4⁺ regulatory T cells (FIGS. 1F and 9A-9E). H&E and Luxol Fast Blue staining of spinal cord sections from mice that received VHH_{MHCII}-MOG₃₅₋₅₅ prior to induction of EAE showed preservation of myelination and less immune cell infiltration (FIGS. 1G and 1H), unlike samples from animals that progressed to EAE. Results indicated that a single 20 μg dose of the VHH_{MHCII}-MOG₃₅₋₅₅ adduct administered prior to induction of EAE was sufficient to prevent the onset of disease.

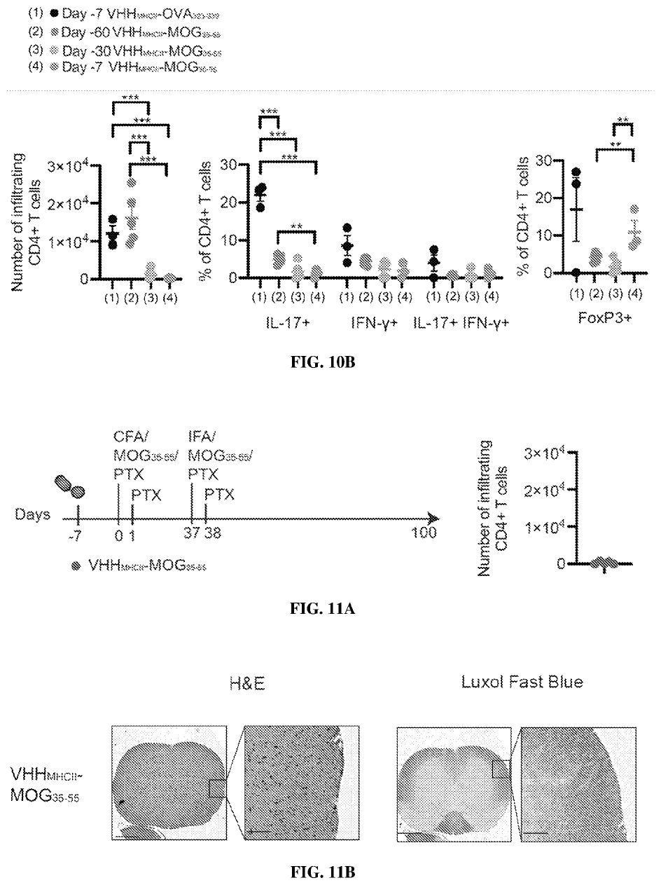

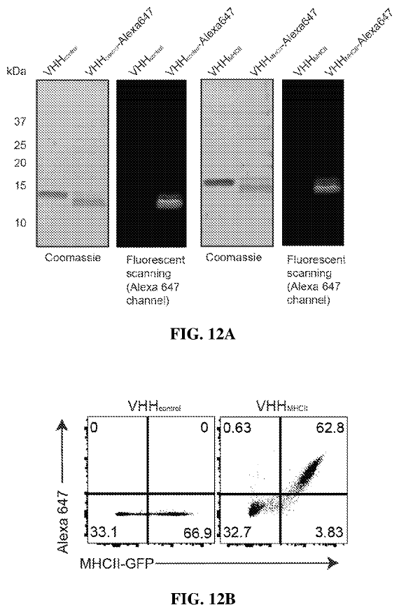

To explore the durability of protection induced by VHH_{MHCII}-MOG₃₅₋₅₅, a single dose of VHH_{MHCII}-MOG₃₅₋₅₅ was administered one or two months prior to induction of EAE with the MOG₃₅₋₅₅/CFA/PTX cocktail. Delayed onset, if not complete suppression of EAE, was observed (FIGS. 11, 10A, and 10B). In spite of the short circulatory half-life of free VHH_{MHCII}-MOG₃₅₋₅₅, estimated to be <0.5 hour, VHH_{MHCII}-MOG₃₅₋₅₅ confers prolonged protection. To explore the extent of resistance to EAE, the protected mice were re-challenged 37 days after the first EAE challenge with a second administration of MOG₃₅₋₅₅/CFA in the presence of PTX. Despite this second highly inflammatory challenge, mice, once protected, showed no signs of developing EAE (FIGS. 1J, 11A, and 11B). Tolerance evoked by a single dose of VHH_{MHCII}-MOG₃₅₋₅₅, even weeks after its administration, thus provides lasting protection.

Splenic CD11c+ DCs are APCs Associated with Induction of Antigen-Specific Tolerance

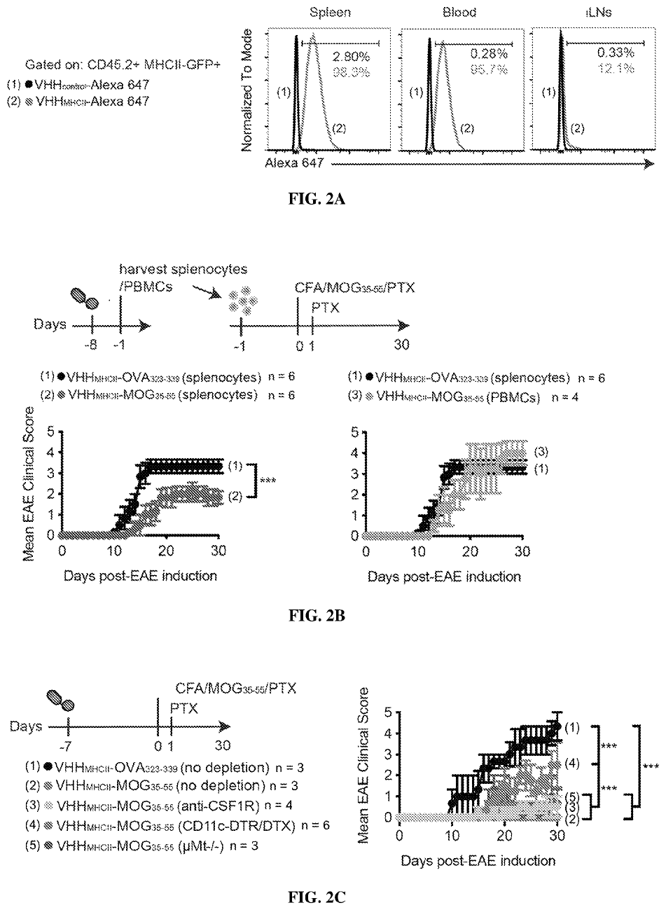

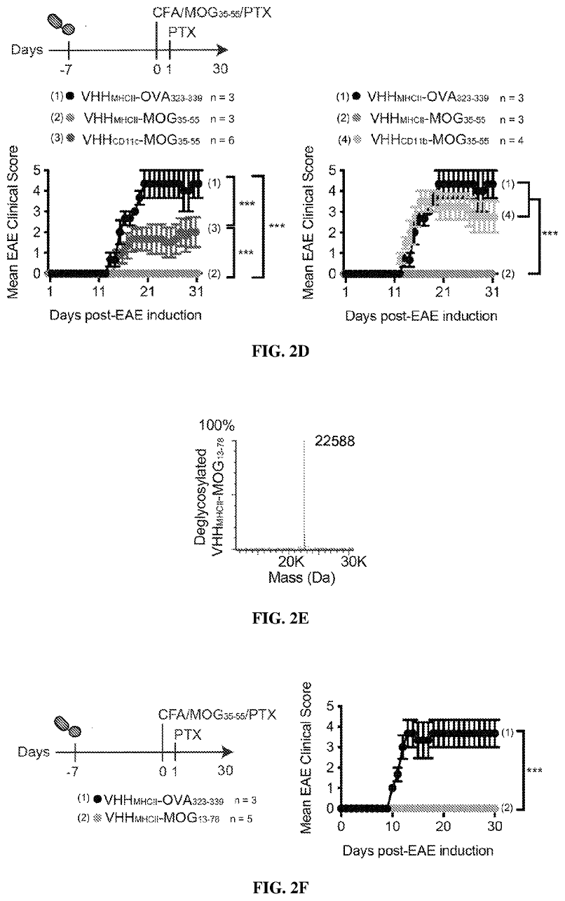

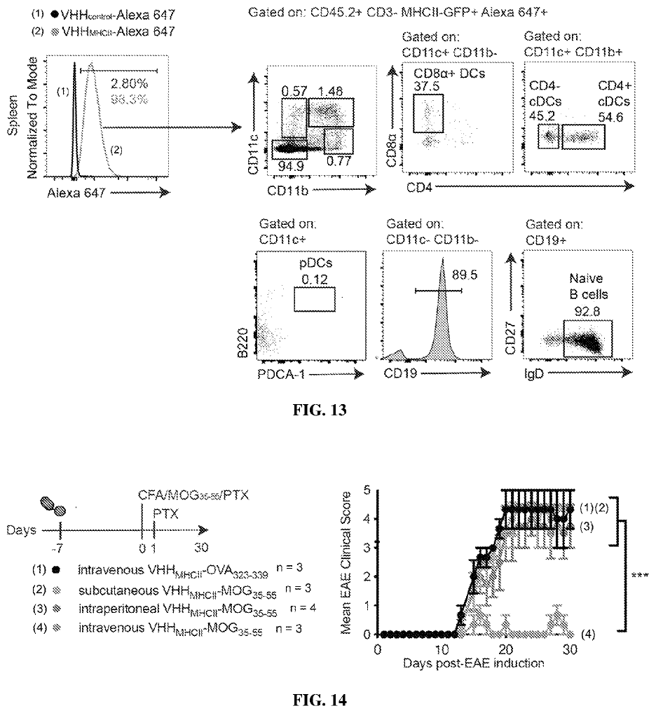

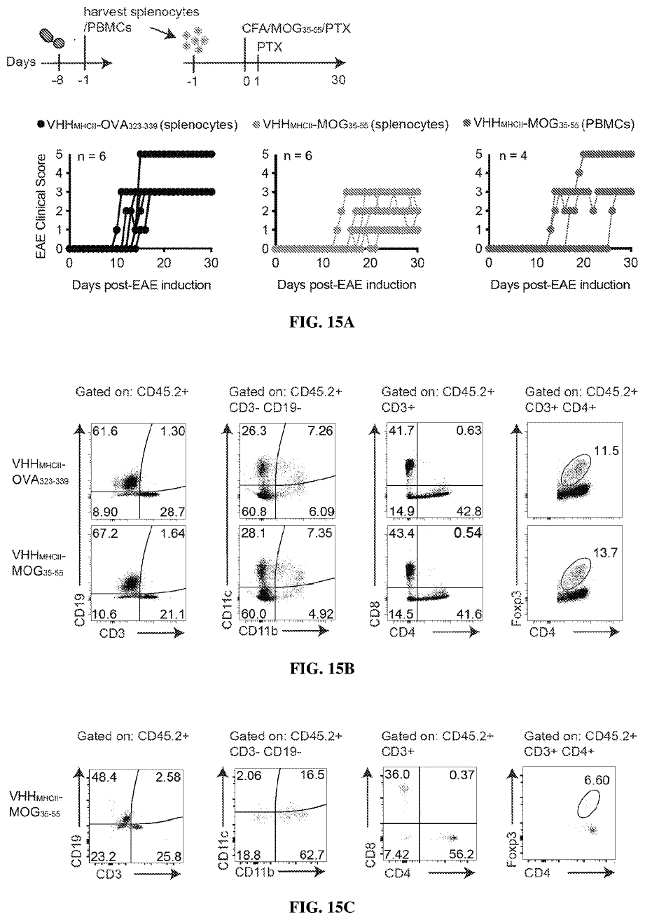

To explore possible mechanisms of VHH_{MHCII}-mediated induction of tolerance, VHH_{MHCII}-Alexa 647 (FIGS. 12A and 12B) was generated and injected (i.v.) into MHCII-GFP mice to follow the biodistribution of VHH_{MHCII}-Alexa647. These mice carry a targeted gene replacement that encodes an I-A^{b}-GFP fusion. It replaces the endogenous I-A^{b} locus and ensures that all Class II MHC+ cells express GFP. At 1.5 hours after injection, VHH_{MHCII}-Alexa647 is captured by a splenic and circulatory MHCII-GFP+ cell population (FIGS. 2A and 13). The fluorescent VHH_{MHCII} adducts were captured by B cells and DC subsets, including splenic CD8a+ DCs, CD4− conventional DCs (cDCs), as well as CD4+ cDCs, but not plasmacytoid DCs (FIG. 13).

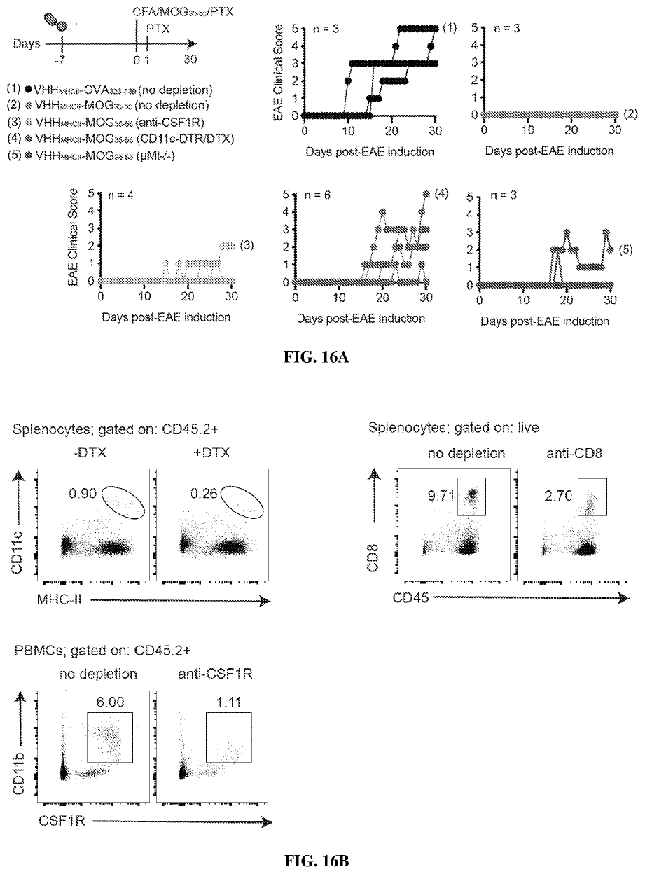

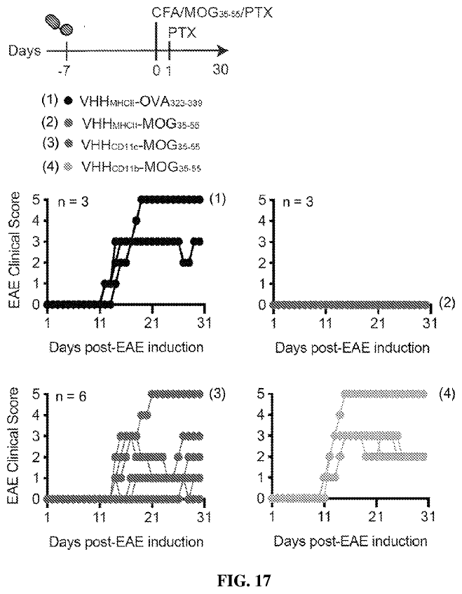

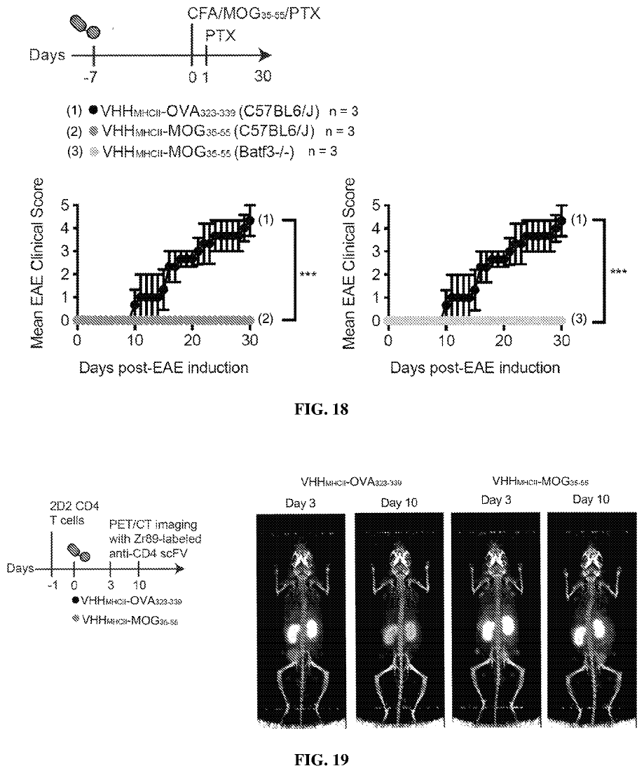

Intravenous, but not subcutaneous or intraperitoneal injection of VHH_{MHCII}-MOG₃₅₋₅₅ protected against induction of EAE (FIG. 14). This hinted at a role of the spleen or the bloodstream as a site of tolerance induction. Therefore 20 μg of VHH_{MHCII}-MOG₃₅₋₅₅ (FIGS. 2B and 15A-15C) were injected (i.v.) into mice and after one week splenocytes and whole blood were harvested as sources of donor cells. Naïve mice then received 20 million unfractionated splenocytes or peripheral blood mononuclear cells (PBMCs) from the VHH_{MHCII}-MOG₃₅₋₅₅ treated animals. One day after cell transfer, MOG₃₅₋₅₅ in CFA+PTX was administered to induce EAE (FIGS. 2B and 15A-15C). There was a significant reduction in the mean clinical EAE score in mice that received splenocytes from mice treated with VHH_{MHCII}-MOG₃₅₋₅₅ (FIGS. 2B and 15A-15C). Macrophages and CD8 T cells were eliminated in vivo by administering the corresponding depleting antibodies: anti-CFS1R antibodies and anti-CD8α antibodies respectively (FIGS. 2C, 16A, and 16B). To deplete DCs, diphtheria toxin (DTX) was administered in CD11c-DTR (diphtheria toxin receptor) mice (FIGS. 2C, 16A, and 16B). To test the possible involvement of B cells, VHH_{MHCII}-MOG₃₅₋₅₅ was administered into μMt-mice, which lack B cells. Only elimination of CD11c+ DCs reduced the measure of protection provided by VHH_{MHCII}-MOG₃₅₋₅₅ (FIGS. 2C, 16A, and 16B). Two VHH-MOG₃₅₋₅₅ adducts were created that presumably target different but overlapping subsets of myeloid cells. These adducts included a VHH directed against CD11b (mostly present on macrophages) and a VHH that recognizes CD11c (mostly present on dendritic cells) (FIG. 8). Only the VHH_{CD11c}-MOG₃₅₋₅₅ combination provided an intermediate level of protection against the induction of EAE (FIGS. 2D and 17), consistent with the results from elimination of CD11c+ cells. Batf3−/− mice treated with VHH_{MHCII}-MOG₃₅₋₅₅ remained resistant to the induction of EAE. In this setting CD8α+ DCs therefore do not obviously contribute to the set of tolerogenic APCs (FIG. 18).

To determine whether delivery by VHH_{MHCII} of more than just the minimal epitope can likewise induce tolerance, VHH_{MHCII}-MOG₁₇₋₇₈ was generated and used to treat mice 7 days prior to challenge. VHH_{MHCII}-MOG₁₇₋₇₈ likewise protected against induction of EAE (FIGS. 2E and 2F).

Administration of VHH_{MHCII}-MOG₃₅₋₅₅ Elicits a Burst of Proliferation, Followed by Attrition, of MOG₃₅₋₅₅-Specific CD4 T Cells.

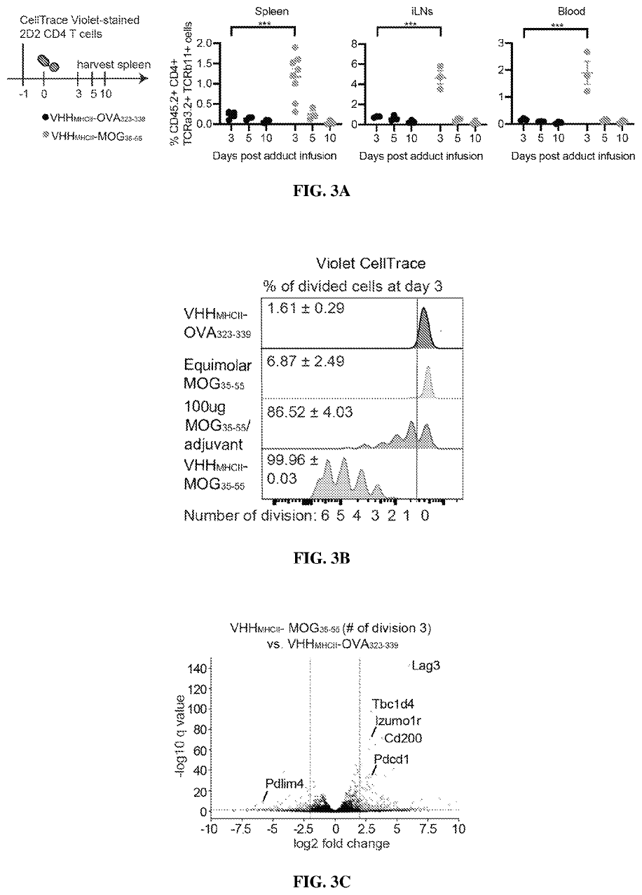

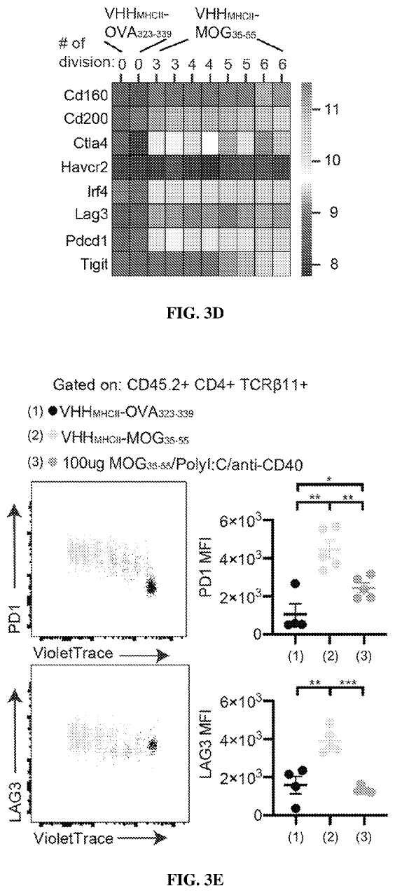

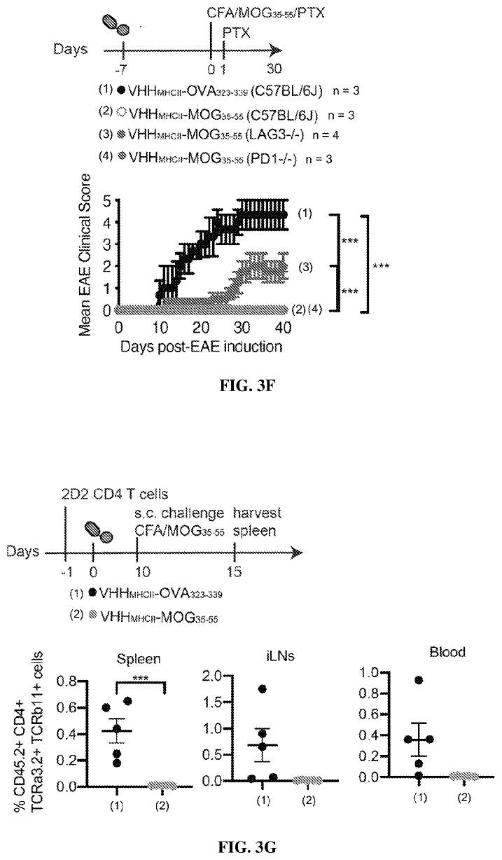

To investigate the impact of VHH_{MHCII}-MOG₃₅₋₅₅ adducts on T cells of defined antigen specificity, 2D2 TCR transgenic mice were used as a source of monoclonal CD4+ T cells that recognize the I-A^{b}-MOG₃₅₋₅₅ complex. Congenically marked, Violet CellTrace-labeled 2D2 CD45.2+ CD4+ T cells were transferred into CD45.1 recipients, followed by injection (i.v.) of VHH_{MHCII}-peptide adducts a day later. The number of 2D2 cells in spleen, inguinal lymph nodes (iLNs), and blood was tracked for 10 days. Mice that received VHH_{MHCII}-MOG₃₅₋₅₅, 2D2 CD4+ T cells underwent an initial burst of expansion, followed by contraction 5 days after injection, as determined by the absolute number of 2D2 cells recovered from spleen, iLNs, and blood as well as whole body imaging using non-invasive positron emission tomography (PET) imaging for CD4+ cells (FIGS. 3A and 19). This disappearance occurred after several cell divisions as all of the recovered 2D2 CD4 T cells were antigen-experienced and had divided, as evinced by Violet CellTrace dilution (FIG. 3B). Delivery of an amount of MOG₃₅₋₅₅ equimolar to that of the administered VHH_{MHCII}-MOG₃₅₋₅₅ adduct led to division of no more than ˜5% of the 2D2 T cells. VHH_{MHCII}-mediated antigen delivery thus clearly enhances its presentation (FIG. 3B).

MOG-Specific 2D2 CD4 T Cells Upregulate Co-Inhibitory Receptors Upon Administration of VHH_{MHCII}-MOG₃₅₋₅₅.

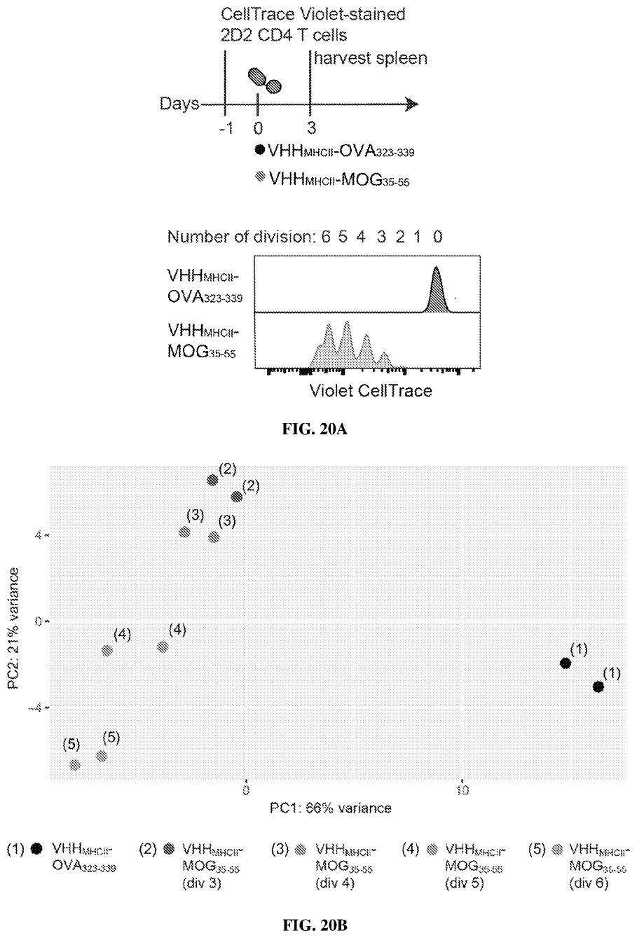

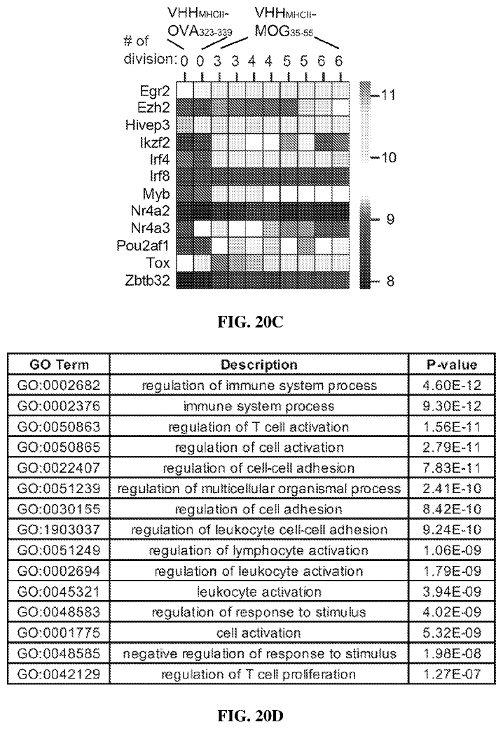

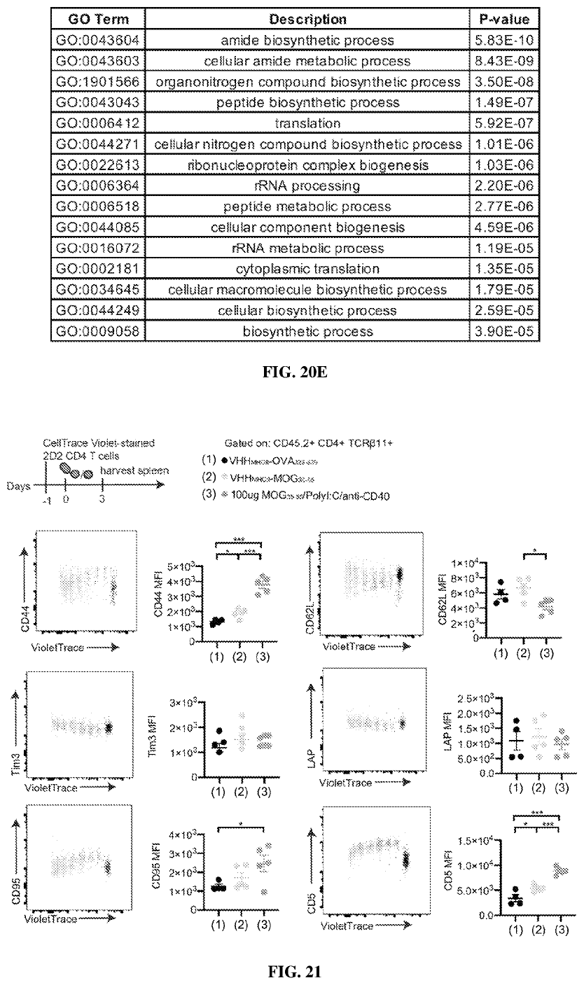

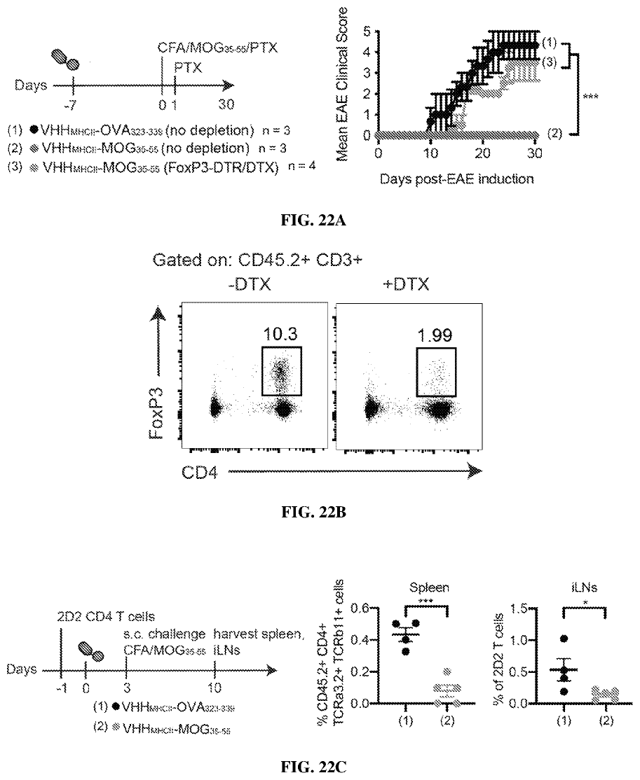

To corroborate these results, the transcriptome of 2D2 T cells in VHH_{MHCII}-MOG₃₅₋₅₅ recipients was examined. 2D2 CD4 T cells at different divisional stages were sorted (FIG. 3B) and RNAseq analyses were performed. Injection of VHH_{MHCII}-MOG₃₅₋₅₅ upregulates co-inhibitory receptor transcripts as well as negative regulatory transcription factors. LAG3 transcripts stand out in both magnitude and significance (FIGS. 3C, 3D, and 20A-20E). At the protein level, these 2D2 T cells also showed higher levels of apoptotic and exhaustion markers, such as PD1 and LAG3, but not Tim3, Fas/CD95, or LAP (FIGS. 3E and 21). At day 3 post-injection, 2D2 CD4 T cells in VHH_{MHCII}-MOG₃₅₋₅₅ recipients perhaps unusually failed to down-regulate CD62L, while remaining CD44+ (FIG. 21). When LAG3−/− mice were treated with a single dose of VHH_{MHCII}-MOG₃₅₋₅₅ and then challenged by induction of EAE, protection was lost, albeit with significant delay, whereas PD1−/− mice were still tolerized by VHH_{MHCII}-MOG₃₅₋₅₅ (FIG. 3F). Deletion of LAG3 in 2D2 TCR transgenic mice leads to spontaneous EAE. Because both activated effector T cells and Tregs express LAG3, whether an increase in regulatory T cells contributes to VHH_{MHCII}-MOG₃₅₋₅₅-imposed tolerance was evaluated.

Administration of VHH_{MHCII}-MOG₃₅₋₅₅ Induces MOG₃₅₋₅₅-Specific Regulatory CD4 T Cells.

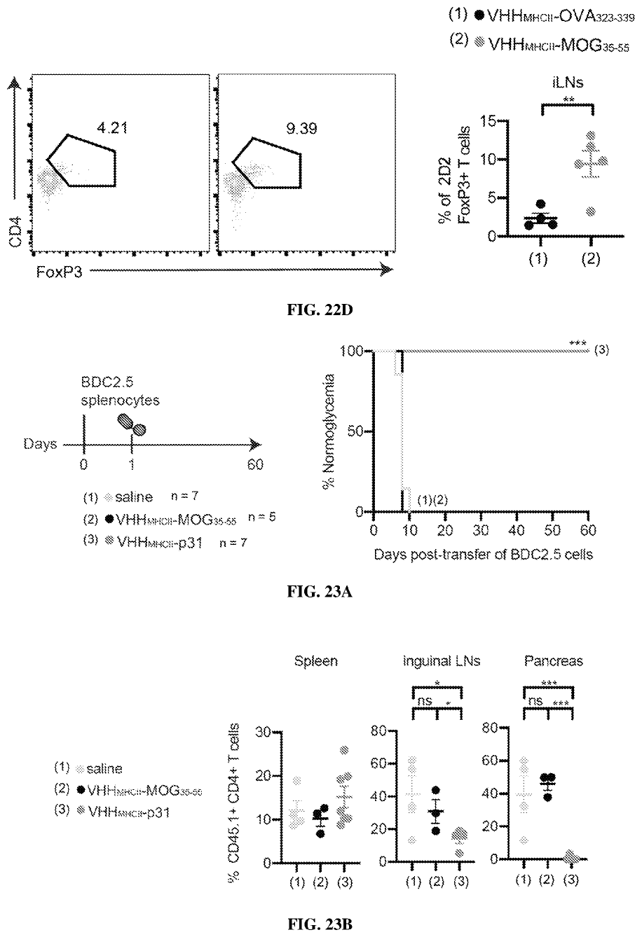

To uncover a role for regulatory T cells in VHH_{MHCII}-MOG₃₅₋₅₅-mediated tolerance, Tregs was eliminated in Foxp3-DTR mice by administration of DTX (FIGS. 22A-22D). Treated mice lost Tregs and were no longer protected against EAE, demonstrating its contribution to VHH_{MHCII}-MOG₃₅₋₅₅-imposed tolerance (FIGS. 22A-22D). Administration of VHH_{MHCII}-MOG₃₅₋₅₅ increases the number of FoxP3+ MOG₃₅₋₅₅-specific Tregs (FIGS. 22A-22D). In addition to the increase in the number of Tregs, expression of exhaustion markers also increased upon administration of VHH_{MHCII}-MOG₃₅₋₅₅. Finally, mice that had received 2D2 T cells were challenged with MOG₃₅₋₅₅/CFA at day 10. The 2D2 T cells in mice that received VHH_{MHCII}-MOG₃₅₋₅₅ failed to respond, whereas 2D2 T cells in mice injected with VHH_{MHCII}-OVA₃₂₃₋₃₃₉ proliferated robustly (FIG. 3G). This underscores the antigen specificity of tolerance induction by VHH_{MHCII}-MOG₃₅₋₅₅.

### VHH_{MHCII}-Antigen Adducts Act in an Antigen-Specific Manner Also in Other Models of Autoimmunity.

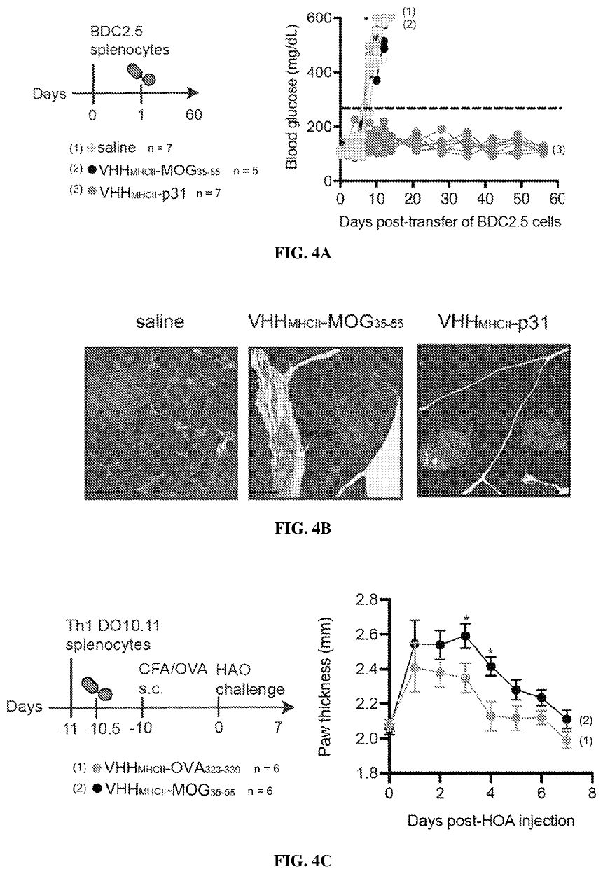

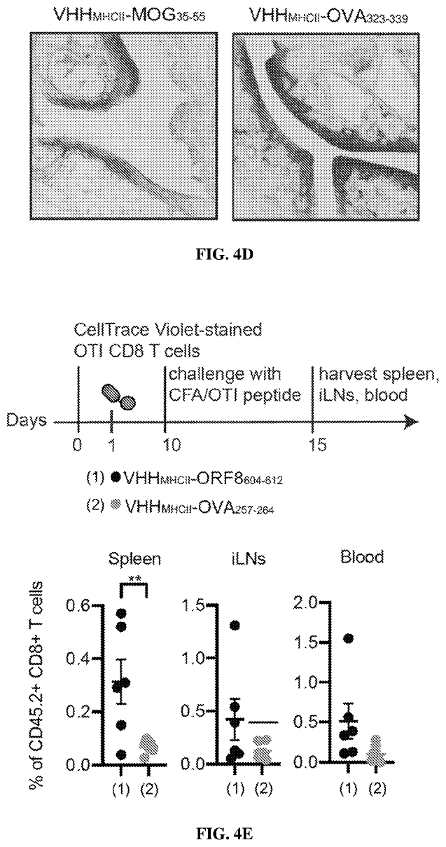

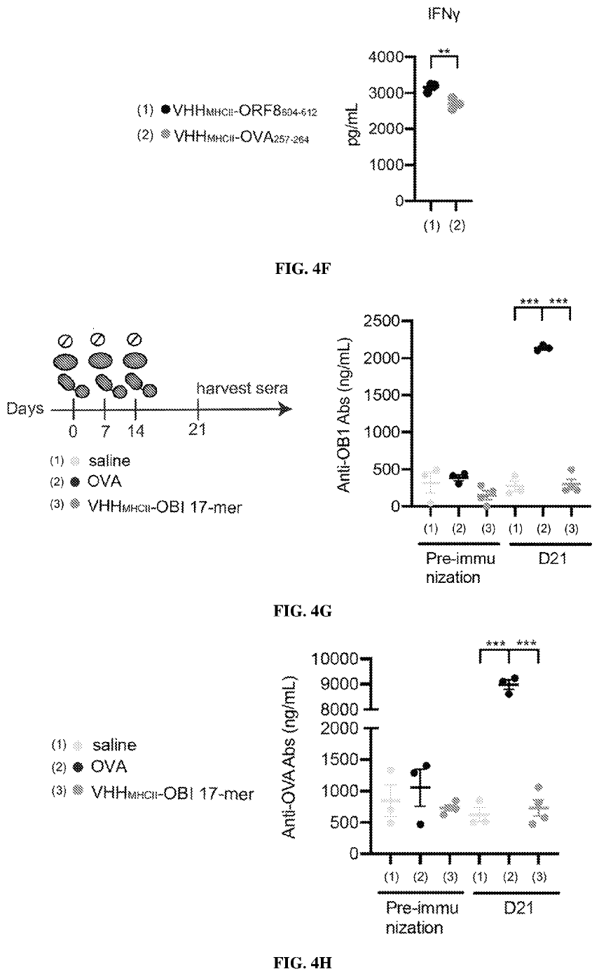

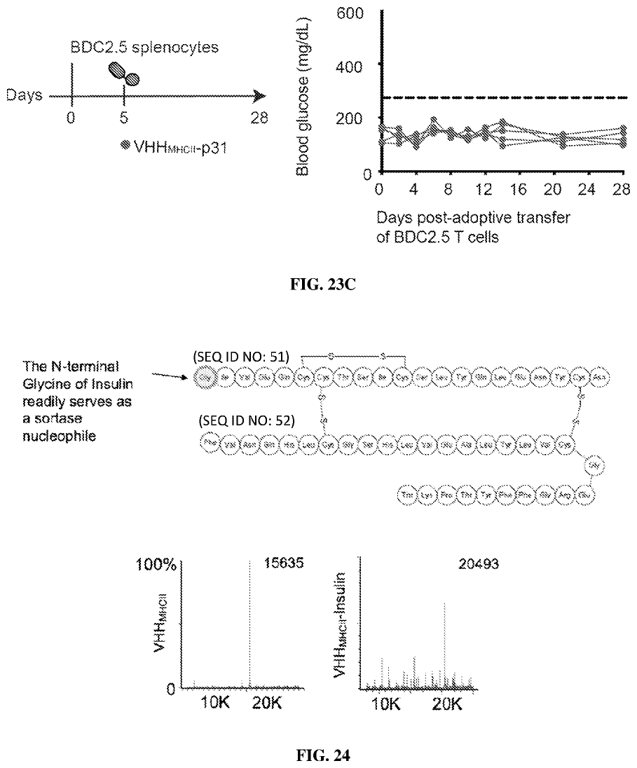

Next, the ability of the VHH-antigen adducts to interfere in other autoimmune conditions was tested. For type-1 diabetes (T1D), the aggressive BDC2.5 T-cell adoptive transfer model that mimics autoreactive T-cell-mediated destruction of β-cells was used. Transgenic CD4 T cells that carry the BDC2.5 T-cell receptor recognize pancreatic β cells and can be activated ex vivo with the mimotope p31. In NOD/SCID mice, such activated BDC2.5 T cells cause hyperglycemia within 8 days after transfer. p31 was conjugated to VHH_{MHCII} (FIG. 8). NOD/SCID mice that received activated BDC2.5 splenocytes were treated a day later with either saline, 20 μg VHH_{MHCII}-MOG₃₅₋₅₅, or 20 μg VHH_{MHCII}-p31 (FIG. 4A). Mice treated with either saline or p31 became hyperglycemic by day 8 post-transfer (FIGS. 4A and 23A-23C). Only mice treated with VHH_{MHCII}-p31 maintained normoglycemia for the duration of the experiment (FIGS. 4A and 23A-23C). VHH_{MHCII}-p31 treated mice had fewer BDC2.5 CD4 T cells in their pancreas and secondary lymphoid organs (FIGS. 23A-23C). Islets in protected mice remained intact (FIG. 4B). There was a mild protective effect even when VHH_{MHCII}-p31 was administered into mice on day 5 post-transfer of the activated BDC2.5 T cells (FIG. 22C). Whole insulin proteins were also attached to VHH_{MHCII} (FIG. 24).

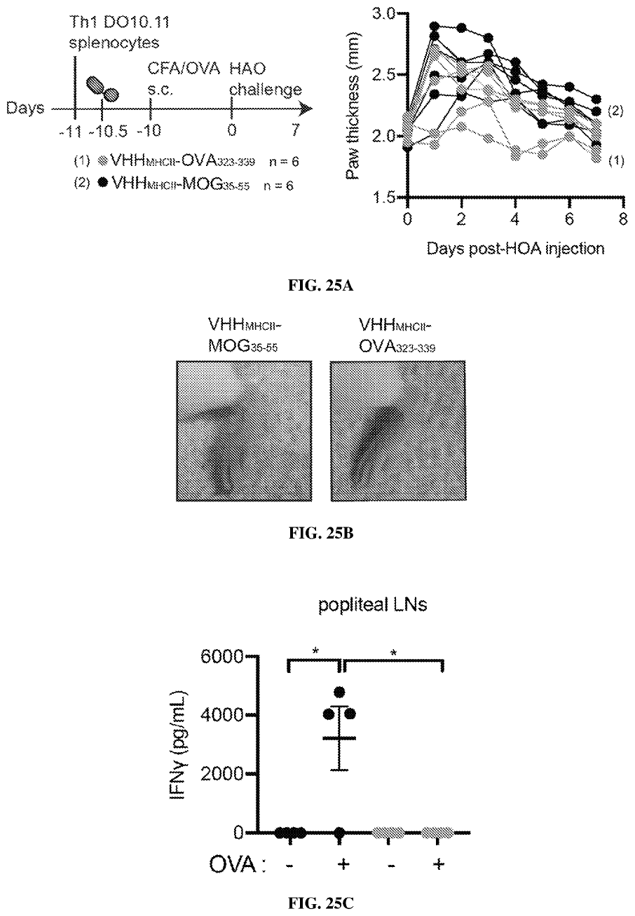

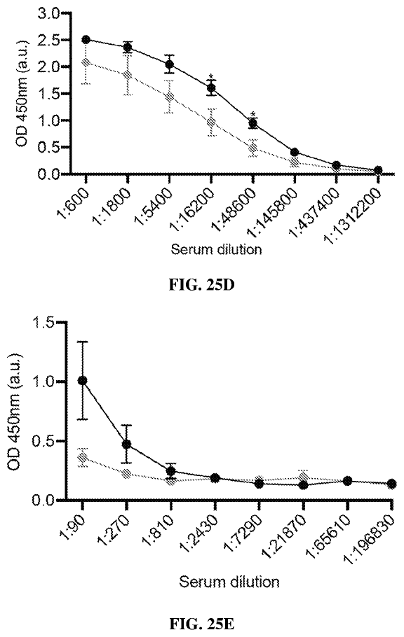

Arthritis can be induced in BALB/c recipients by intravenous transfer of ex vivo activated Th1 DO11.10 T cells that recognize OVA₃₂₃₋₃₃₉, followed one day later by a footpad injection of OVA/CFA emulsion and a challenge 10 days later by heat-aggregated ovalbumin (HAO) (FIG. 4C). Mice were then monitored for development of arthritis by measuring paw thickness and by histological assessment at day 7 following challenge with HAO. Prior administration of VHH_{MHCII}-OVA₃₂₃₋₃₃₉ reduced joint inflammation upon exposure to ovalbumin, whereas VHH_{MHCII}-MOG₃₅₋₅₅ had no effect (FIGS. 4C and 25A-25E). Mice treated with VHH_{MHCII}-OVA₃₂₃₋₃₃₉ also showed fewer signs of cartilage destruction (FIG. 4D). Immune cells obtained from popliteal lymph nodes of mice treated with VHH_{MHCII}-OVA₃₂₃₋₃₃₉ failed to produce IFNγ when stimulated ex vivo with OVA (FIGS. 25A-25E). Perhaps not unexpectedly, serum from mice treated with VHH_{MHCII}-OVA₃₂₃₋₃₃₉ also had lower levels of anti-OVA and anti-OVA₃₂₃₋₃₃₉ IgG1 antibodies (FIG. 25A-25E).

Combined, these results confirm the ability of VHH_{MHCII}-antigen adducts to reduce the harm inflicted by activated, autoreactive CD4 T cells. The underlying mechanism(s) must be conserved across mouse MHC haplotypes.

### VHH_{MHCII}-Antigen Adducts Also Suppress CD8-Mediated T and B Cell Responses.

To determine whether CD8 T cell responses are affected by administration of VHH_{MHCII}-antigen adducts, the OVA-derived CD8 T cell epitope SIINFEKL (the OTI peptide restricted by H-2K^{b}) was attached to VHH_{MHCII} (FIG. 8). Mice received congenically marked OTI T cells, followed by injection of VHH_{MHCII}-OTI or VHH_{MHCII}-ORF8 (with or without adjuvant) a day later (FIG. 4E). The ORF8 epitope derived from MCMV is recognized by CD8 T cells in H-2^{b} mice and served as a control. A re-challenge of the recipients with OVA/CFA at day 10 post transfer failed to activate any remaining OTI T cells (FIG. 4F). To explore whether B cell responses are similarly affected by administration of VHH_{MHCII}-antigen adducts, VHH_{MHCII} was modified with a B cell-specific OVA-derived epitope (OB1) (FIG. 8). Three consecutive injections of VHH_{MHCII}-OBI into C57BL/6J recipients failed to elicit IgG antibody responses against either intact OVA protein or the OB1 peptide (FIGS. 4G and 4H), whereas mice that received equimolar amounts of free OVA protein readily produced such antibodies.

Co-Delivery of VHH_{MHCII}-MOG₃₅₋₅₅ and VHH_{MHCII}-Dexamethasone Increases Therapeutic Efficacy.

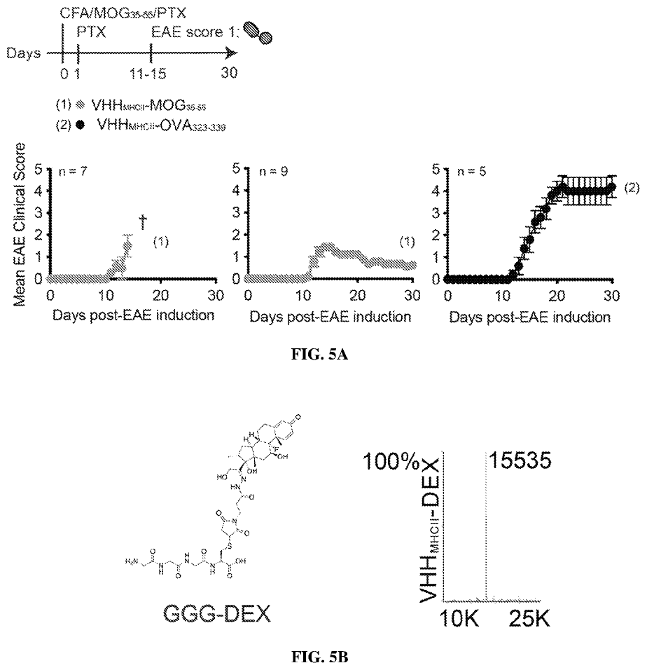

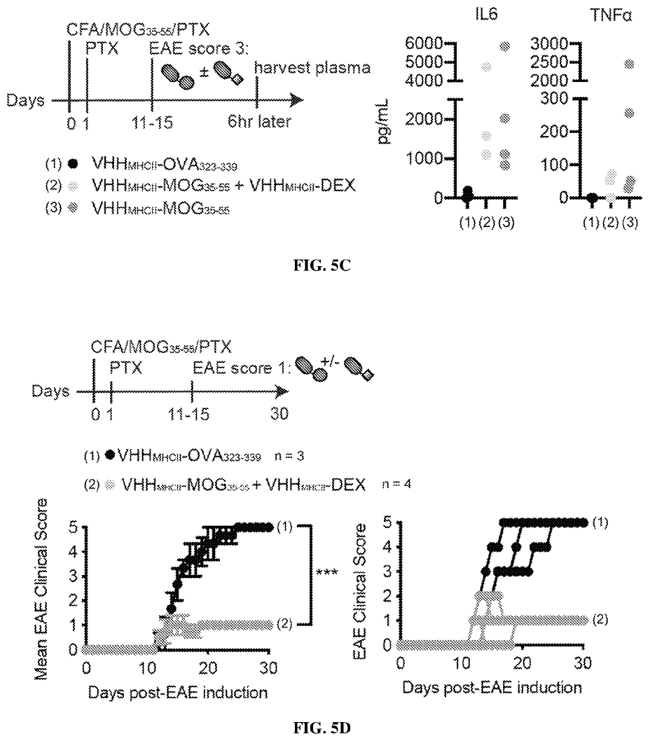

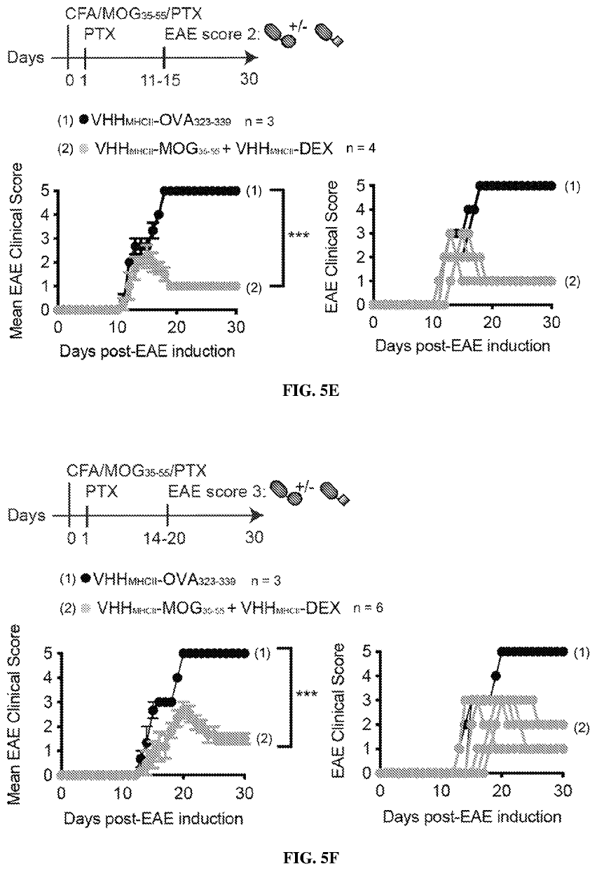

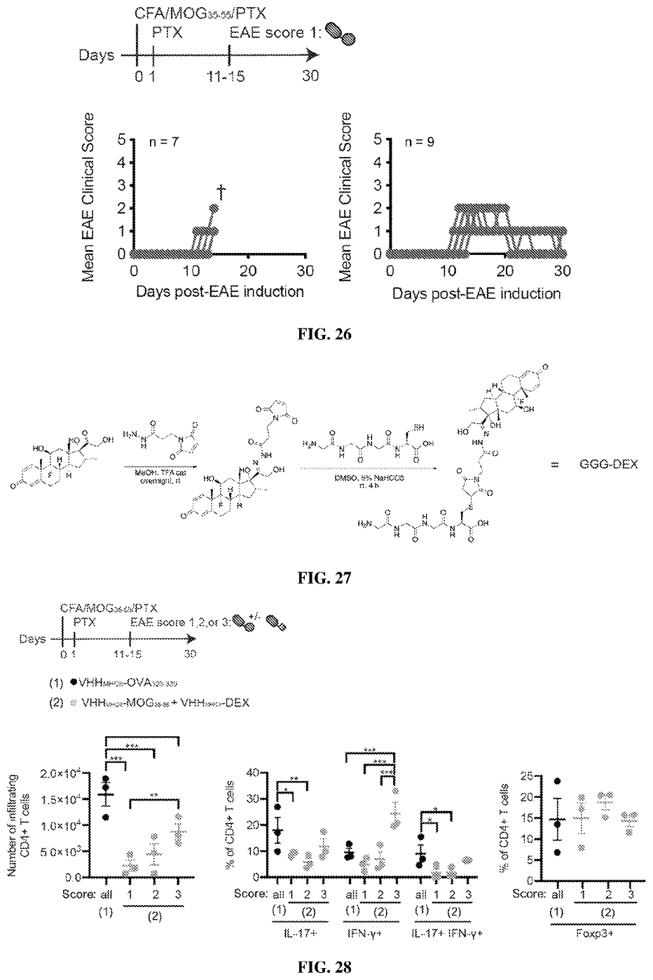

The impact of VHH_{MHCII}-MOG₃₅₋₅₅ administration to mice already symptomatic for EAE was then explored. Injection of VHH_{MHCII}-MOG₃₅₋₅₅ into mice that had developed a clinical score of 1 (limp tail), halted progression of EAE in 9 out of 16 mice (FIGS. 5A and 26). The overall condition of the remaining 7 out of 16 mice rapidly deteriorated (e.g. shivering; reduced motor activity) after injection of VHH_{MHCII}-MOG₃₅₋₅₅, seemingly unrelated to EAE. In fact, ˜40% of mice receiving VHH_{MHCII}-MOG₃₅₋₅₅ were dead the day after infusion, without correlation to the clinical score of the mice prior to injection. A cytokine storm elicited by the targeted delivery of antigen into an already inflamed environment was responsible, as indicated by elevated levels of IL-6 and TNFα. (FIG. 5C).

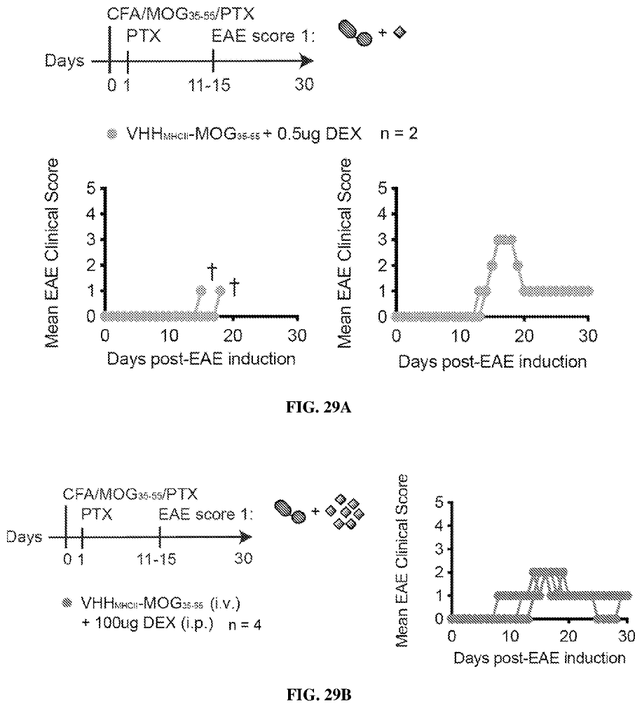

The polyclonal nature of the evoked T cell response and the rather superficial clinical scoring system imply heterogeneity in the diseased cohort, which may explain why not all animals that received VHH_{MHCII}-MOG₃₅₋₅₅ responded similarly. It was then tested whether it might be possible to co-deliver an immunosuppressive drug to avert a cytokine storm. The immunosuppressive corticosteroid dexamethasone, attached via a self-hydrolyzing hydrazone linker to VHH_{MHCII}, was delivered to Class II MHC+ cells (VHH_{MHCII}-DEX; FIGS. 5B and 27). Mice that received a combined dose of 20 μg VHH_{MHCII}-MOG₃₅₋₅₅ and 20 μg VHH_{MHCII}-DEX survived and reverted to lower clinical EAE clinical scores, without obvious side effects (FIG. 5D). Improvements in clinical score were mirrored by a reduction in infiltrating CD4 T cells in the spinal cord (FIG. 28). The observed benefit required no more than the equivalent of 0.5 μg DEX in the form of the VHH_{MHCII}-DEX adduct. Free DEX, on the other hand, provided protection only when administered at a ˜200-fold higher dose of 100 μg i.p. (FIGS. 29A and 29B). The therapeutic range was extended to animals that had progressed to an EAE score of 2 or 3, all of which responded to co-administration of VHH_{MHCII}-MOG₃₅₋₅₅ and VHH_{MHCII}-DEX by an arrest in disease progression, again without side effects. Affected mice even showed a significant amelioration in disease score (FIGS. 5E, 5F, and 28). Surprisingly, the route of administration is important, as only intravenous, not subcutaneous or intraperitoneal, delivery of VHH_{MHCII}-DEX could provide prophylactic protection (FIG. 14).

### Anti-Human MHCII VHH (VHH_{hMHCII})-Antigen Adducts in Humanized Mouse Models of Autoimmune Disease

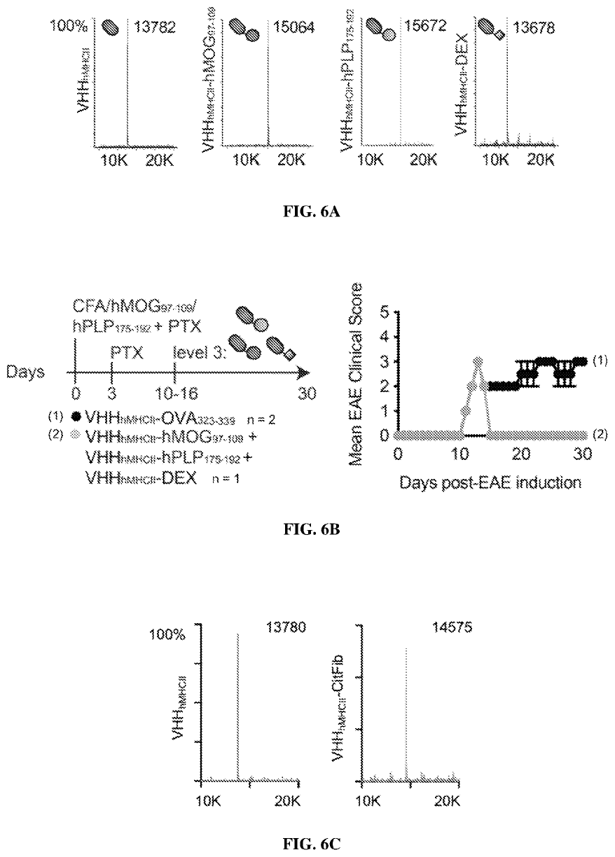

A VHH that recognizes a wide range of human Class II MHC molecules (VHH_{hMHCII}) was developed. This VHH was prepared in a sortase-ready format and modified with several self-antigens of human origin (FIGS. 6A-6C).

Human MOG₉₇₋₁₀₈ peptide (TCFFRDHSYQEE (SEQ ID NO: 53)), hPLP₁₇₅₋₁₉₂ peptide (YIYFNTWTTCQSIAFPSK (SEQ ID NO: 42)) and DEX to VHH_{hMHCII} were attached (FIG. 6B). The efficacy of these adducts co-delivered in the HLA-DR4-IE-transgenic C57BL/6 IAb^{null} mice which lack murine MHC-II but instead express a transgenic hybrid MHC-II molecule composed of the peptide-binding domain of human HLA-DR4 and the membrane-proximal domain of mouse IE (DR4-IE) was tested. VHH_{hMHCII}-MOG₉₇₋₁₀₈ reduced the EAE clinical score in mice 20 days after administration (n=1, FIG. 6B). VHH_{hMHCII}-OVA₃₂₃₋₃₃₉ was used as negative control (n=2, FIG. 6B)

A frequent target of autoantibodies in RA patients are post-translationally modified antigens such as Fibrinogen a that carry citrulline, a modified arginine residue. Hence, VHHₕcu was modified with citrullinated Fibα₇₉₋₉₁ (QDFTNCitINKLKNS (SEQ ID NO: 50), FIG. 6C). It illustrated the flexibility of the chemoenzymatic approach, which—unlike genetic methods—readily allows incorporation of non-natural or post-translationally modified amino acids in site specific manner.

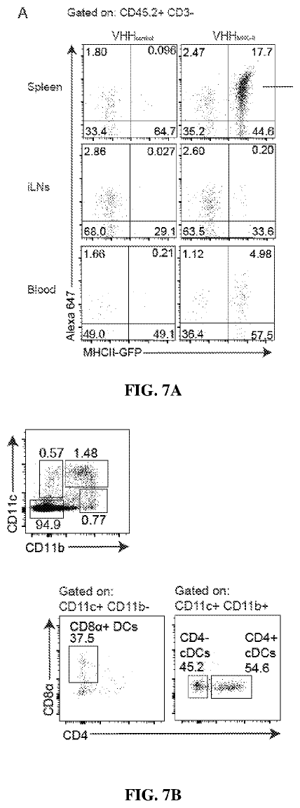

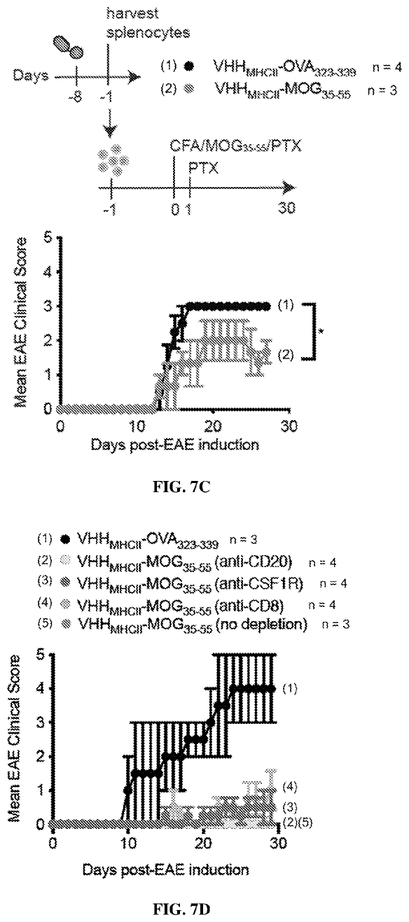

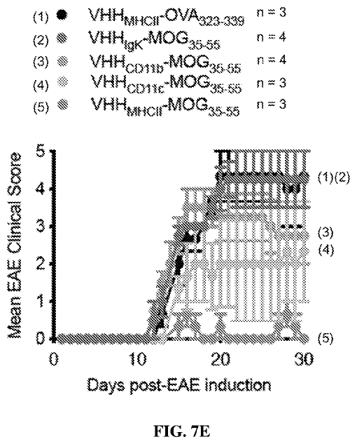

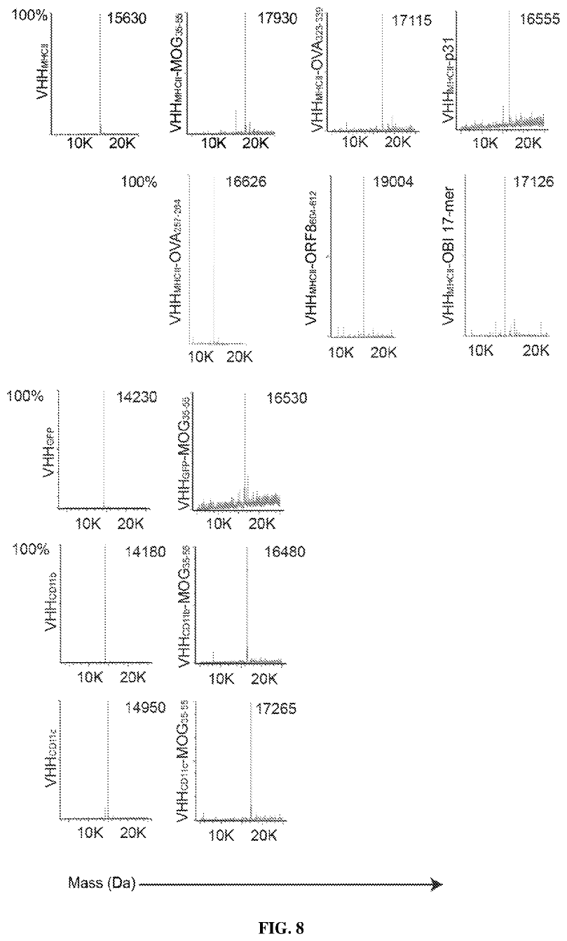

To explore the mechanism of VHH_{MHCII}-mediated tolerance induction, VHH_{MHCII}-Alexa647 was constructed to follow the biodistribution of the VHH_{MHCII} adducts. 20 μg VHH_{MHCII}-Alexa647 was administered intravenously to Class II MHC-GFP mice. At 1.5 hours after injection, the majority of VHH_{MHCII}-Alexa647 was captured by a splenic MHCII-GFP+ cell population in vivo (FIG. 7A). VHH_{MHCII} adducts were delivered to multiple subsets of DCs including splenic CD8α+ DCs, CD4-negative conventional DCs, as well as CD4+ conventional DCs (FIG. 7B).

In the EAE model intravenous, but not subcutaneous or intraperitoneal, injection of VHH_{MHCII}-MOG₃₅₋₅₅ conferred protection against induction of EAE (FIG. 14). 20 mg of VHH_{MHCII}-MOG₃₅₋₅₅ was then injected (i.v.) into mice and one week later their splenocytes were harvested and 20 million total splenocytes were transferred into a cohort of recipient mice. One day after transfer, EAE was induced (FIG. 7C). There was a significant reduction in the mean clinical EAE score, demonstrating that even unfractionated splenocytes induced VHH_{MHCII}-MOG₃₅₋₅₅-mediated tolerance (FIG. 7C). Depletion experiments were performed to target splenic APC subsets. B cells, macrophages, and dendritic cells were depleted by administering the corresponding depleting agents, anti-CD20 antibodies, anti-CFS1R antibodies, and diphtheria toxin (DTX), respectively, into CD11c-DTR (diphtheria toxin receptor) mice (FIG. 7D). Three different VHHs that presumably APC target different but overlapping subsets of APCs were identified: CD11b (mostly present on macrophages), CD11c (mostly present on dendritic cells), and Igk (B cells). These VHHs were expressed in sortase-ready format and were labeled site-specifically with GGG-MOG₃₅₋₅₅ using sortase (FIG. 8). Only VHH_{CD11c}-MOG₃₅₋₅₅ provided an intermediate level of protection against induction of EAE. This suggested a role for CD11c+ cells as tolerogenic APCs (FIG. 7E).

### Discussion

The induction of antigen-specific tolerance is an aspirational goal in the treatment of auto-immune diseases. This is a particularly high bar to clear if one considers the presence of pathology and pre-existing autoimmunity at diagnosis. Auto-immune destruction of target cells is already well on its way before symptoms arise. Therapy must therefore deal not only with existing autoimmunity but also with the possibility of epitope spreading beyond the initiating insult. Any type of prophylactic treatment will be of limited value unless susceptible populations can be unambiguously identified, and then only if the risk of eliciting unwanted side effects is acceptably small.

In addition to curbing inflammation, wholesale immunosuppression has been the backstop in the treatment of autoimmunity, which comes with an increased risk of infectious disease. While antibiotic treatment can mitigate this drawback at least in part, the search for a more targeted approach to blunt undesirable immune reactions remains a priority. Most auto-immune diseases are T cell-mediated; T cell activation involves professional antigen presenting cells. If antigen presenting cells (APCs) acquire antigen in an inflammatory environment, upregulation of costimulatory molecules as well as the production of the proper mix of cytokines contribute to T cell activation. Tolerogenic dendritic cells are devoid of such costimulatory signals and consequently antigen presentation under non-inflammatory conditions promotes a state of non-responsiveness or tolerance. This concept has driven the exploration of tolerogenic dendritic cells. Dendritic cells can be sub-divided into subsets with distinct functional capacities, for example the ability to engage in antigen cross-presentation is a property mostly ascribed to the DC1 subset. The identification of surface receptors involved in antigen acquisition has identified DEC205, DC-SIGN and Clec9a as particularly relevant for entry of antigen into cross-presentation pathways. While pursued primarily as strong inducers of desirable immunity, such as anti-tumor responses, their ability to induce regulatory T cells as a means of reducing unwanted responses is considered no less important.

This rather narrow focus on dendritic cells has overshadowed earlier work in which antigens were targeted to Class H MHC products, expressed on all antigen-presenting cells, through the creation of anti-Class II MHC antibodies conjugated to self-antigens, It is, after all, the Class H MHC peptide complex that is the call to arms for the CD4 T cell compartment. For this reason, autoantigens were delivered under non-inflammatory conditions to Class II MHC-positive cells, a strategy that does not differentiate among the various APC subsets, but is efficacious nonetheless. Ideally, interventions ought to be antigen-specific and as simple as possible, both from a manufacturing and application perspective.

This data establishes that a MOG₃₅₋₅₅-modified VHH that recognizes Class H MHC products can protect mice against the induction of EAE. A single injection of 10 micrograms of the adduct afforded protection that lasted for at least two months following administration of the nanobody-peptide adduct. Administration of the same VHH_{MHCII}-MOG₃₅₋₅₅ adduct in animals that already show symptoms of EAE (score 1, 2, or 3) halted progression, and even partially reversed the severity of the symptoms. When treating animals with EAE symptoms, only a subset responded, whereas the remainder showed rapid exacerbation, followed by death attributable to a cytokine storm. In symptomatic animals an inflammatory environment already exists, and delivery of the VHH_{MHCII}-MOG₃₅₋₅₅ adduct to APCs only added fuel to the fir. To overcome this acute response, a VHH_{MHCII}-dexamethasone adduct was co-delivered, which dramatically improved survival, with no deaths.

Administration of nanobody-peptide adducts in the presence of anti-CD40 and poly dIdC as adjuvants strongly potentiated antibody responses against them. Administration in a setting where there is a chronic inflammatory response would be possible only if appropriate countermeasures were available, as in the case of the VHH_{MHCII}-dexamethasone adduct.

The pharmacokinetic properties of nanobodies make them attractive for the construction of antibody-drug conjugates (ADCs). Nanobodies have a much shorter circulatory half-life than full sized antibodies, thus minimizing systemic exposure to compounds that are toxic. Their targeting properties are excellent, ensuring that once on site, self-immolating linkers will release the payload predominantly at the intended site. Full-sized immunoglobulin-based ADCs continue to circulate for periods up to weeks and release payloads directly into the bloodstream upon hydrolysis of the linkers via which the drugs are attached. The VHH_{MHCII}-dexamethasone adduct thus has the desired properties of excellent targeting, as verified by non-invasive imaging, short circulatory half-life and ease of modification. The cellular targets recognized by VHH_{MHCII} include all Class II MHC-positive cells. Even if the APCs responsible for induction of tolerance and for provoking a cytokine storm are distinct, the Class II MHC-based targeting approach would obviously cover both. Nanobody-drug adducts have yet to find the broad range of applications of their full-sized counterparts, but these data show it is an opportunity not to be discounted.

As to the mechanism that underlies the remarkable ability of anti-Class II nanobodies to induce tolerance against an attached payload, many possibilities can be excluded, based on the response seen in knock out mice or upon depletion of certain sets of cells. It is still unknown whether a single type of APC can be tolerogenic if targeted under non-inflammatory conditions, while provoking a strong response if antigen is encountered in an inflamed environment.

### Example 2. VHH_{MHCII}-Antigen Fusion Protein as an Efficacious Vaccine

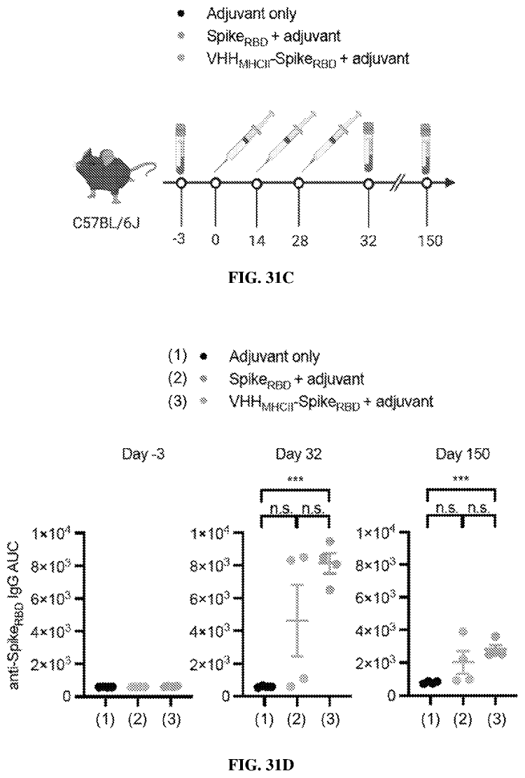

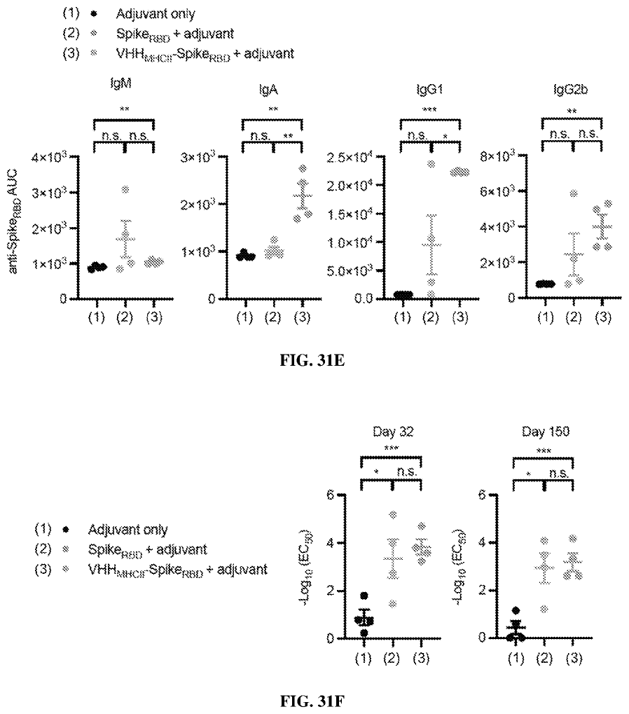

A single-domain antibody fragment (nanobody or VHH) that binds MHC class II antigens (VHH_{MHCII}) was isolated and characterized with nanomolar affinity. To adapt this vaccine platform for SARS-CoV-2, a recombinant protein consisting of a fusion between VHH_{MHCII} and the SARS-CoV-2 receptor-binding domain was generated (VHH_{MHCII}-Spike_{RBD}) (FIGS. 31A-31B).

To confirm the immunogenicity of VHH_{MHCII}-Spike_{RBD}, C57BL/6J mice were intraperitoneally primed with 20 ug of adjuvanted (poly dIdC and anti-CD40 monoclonal antibody) Spike_{RBD}, adjuvanted VHH_{MHCII}-Spike_{RBD}, or adjuvant alone and were subsequently boosted with the homologous vaccine at post-prime as indicated (FIG. 31C). Serum was collected from all animals at days 32 and 150 and IgG titers were determined against recombinant SARS-CoV-2 Spike_{RBD} by ELISA (FIG. 31D). Immunization with the VHH_{MHCII}-Spike_{RBD} fusion consistently produces higher titers of antigen-specific IgG as compared to Spike_{RBD}, which displayed varied immune responses. Not unexpectedly, titers of circulating IgG drop after the day 32 sample, but even at day 150, readily detectable titers against the Spike_{RBD} persist for all mice that received VHH_{MHCII}-Spike_{RBD} Or Spike_{RBD}. Even at day 150, antibody titers in mice receiving the VHH_{MHCII}-Spike_{RBD} fusion still outperform those in the Spike_{R}BD-only cohort. Predictably, pre-immune sera or sera obtained from mice that received adjuvant only did not show significant anti-Spike_{RBD} antibody production. Immunoglobulin subclass analysis revealed evidence of class-switching, as high levels of IgA, IgG1, and IgG2b were detected 32 days post-initial dose (FIG. 31E). Particularly noteworthy is the stronger IgA response evoked by the VHH_{MHCII}-Spike_{RBD} fusion, which would provide mucosal protection important for a respiratory tract infection. A much stronger IgG1 response is also evident, relevant for complement-mediated lysis of opsonized cells.

A functional correlate of serological response was next evaluated by assaying the neutralization capacity of the resulting sera against vesicular stomatitis virus (VSV) pseudo-typed with the SARS-CoV-2 Spike glycoprotein. The sera obtained from mice immunized with the VHH_{MHCII}-Spike_{RBD} fusion outperformed those from mice immunized with Spike_{RBD} only (FIG. 31F). The latter show considerable mouse to mouse variation, whereas the response from mice that received VHH_{MHCII}-Spike_{RBD} is both stronger and more consistent between individual mice, highlighting the importance of direct targeting of antigen presenting cells (APCs). This is in accordance with the level of humoral immune response detected.

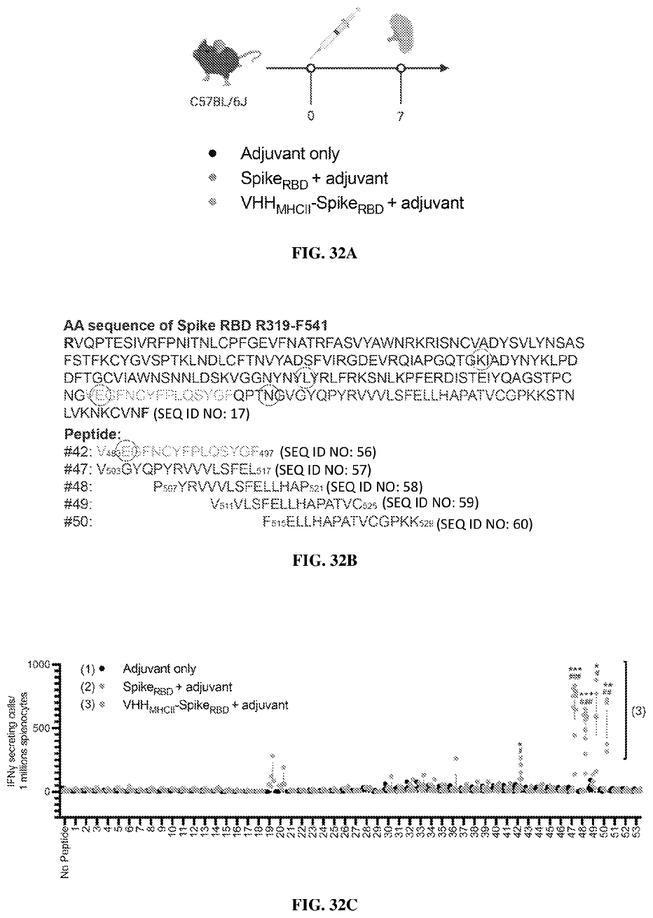

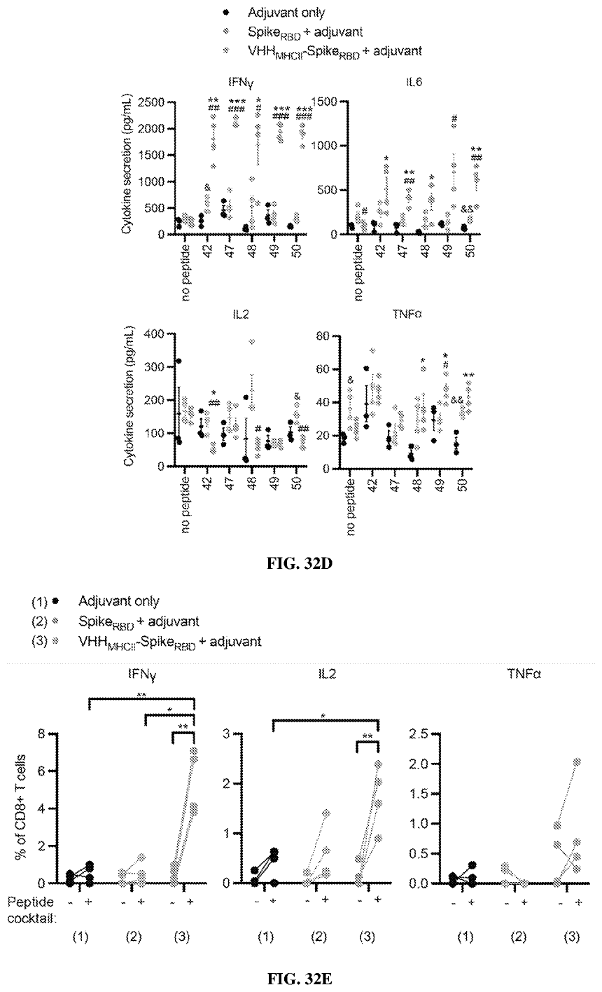

A robust CD8+ T cell response is important for the clearance of virus infected cells. Therefore, mice were immunized with a single dose of either VHH_{MHCII}-Spike_{RBD} or Spike_{RBD}, each in the presence of adjuvant (FIG. 32A). One week later splenocytes were harvested and an ELISpot assay was conducted to identify peptides capable of eliciting IFNγ production in vitro as a surrogate measurement of specific T cell response. Overlapping 15-mer peptides were used and 5 peptides (42, 47, 48, 49, and 50) were identified that elicited a strong response (FIGS. 32B-32D). Mice that were immunized with Spike_{RBD} only recognize some of the peptides and with a much weaker signal than those immunized with VHH_{MHCII}-Spike_{RBD}, indicating a fewer number of cells secreting IFNγ. These results also indicate at least 2 stimulatory regions. Interestingly, peptides 47-50 fall within a Spike_{R}BD region outside of known mutations of the circulating SARS-CoV-2 variants. Cytokine secretion assays against IFNγ, IL6, IL2, and TNFα upon co-culturing splenocytes with selected Spike_{RBD} peptides (42, 47, 48, 49, and 50) further corroborates a superior T cell response elicited by VHH_{MHCII}-Spike_{RBD} (FIG. 32E).

To distinguish between CD4+ and CD8+ T cells as the source of IFNγ, a flow cytometry assay was conducted, followed by intracellular cytokine staining. Most of the inflammatory cytokines were observed to arise from a CD8+ T cell response, based on the incubation of splenocytes with a mixture of peptides 42, 47, 48, and 49 (FIG. 32E). Therefore, VHH_{MHCII}-antigen adducts can enhance cross-presentation and induce an efficacious CD8+ T cell response against Spike_{RBD}.

Moreover, a strong CD8+ T cell response is observed with merely a single immunization and arises within 7 days post-immunization. This strongly suggests that VHH_{MHCII}-Spike_{RBD} is capable of providing protective immunity against the SARS-CoV-2 infection, as the immunized cohort demonstrates a T cell response relatively early while waiting for the slower humoral response to emerge. Together, these data indicate the superiority of directly targeting APCs via Class II MHC.

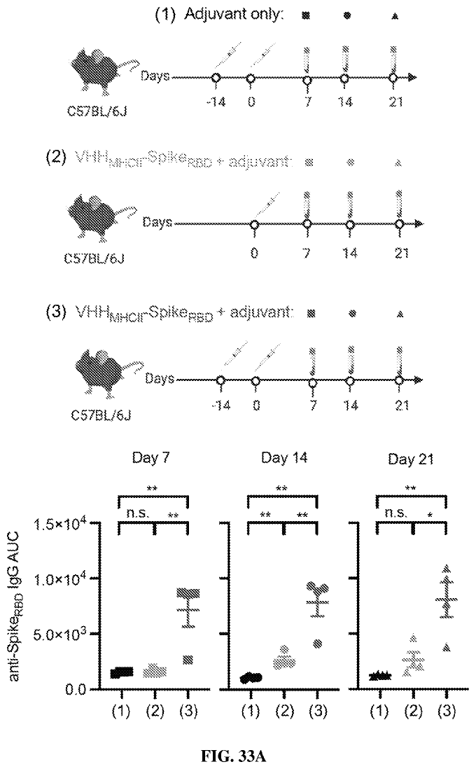

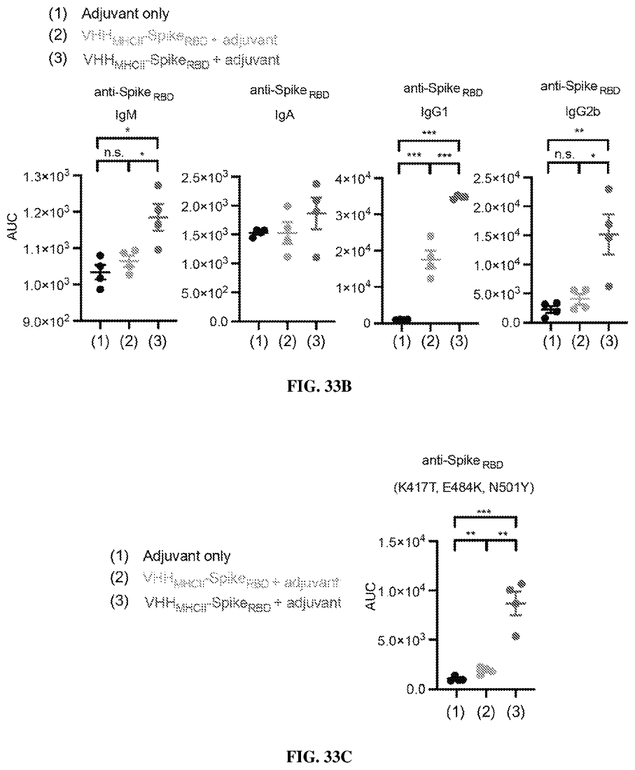

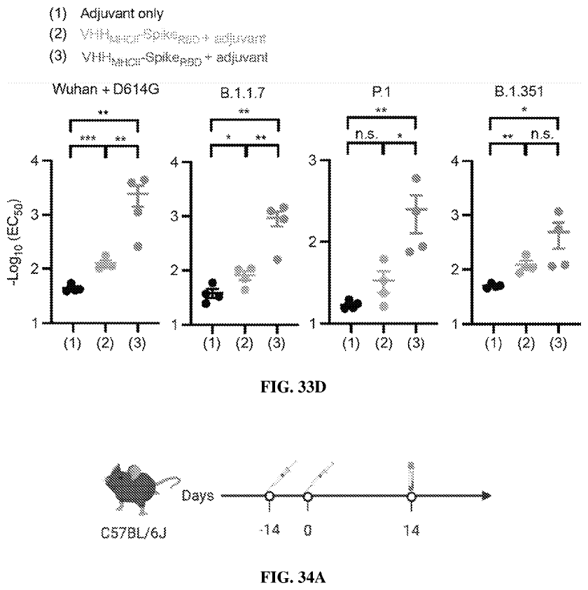

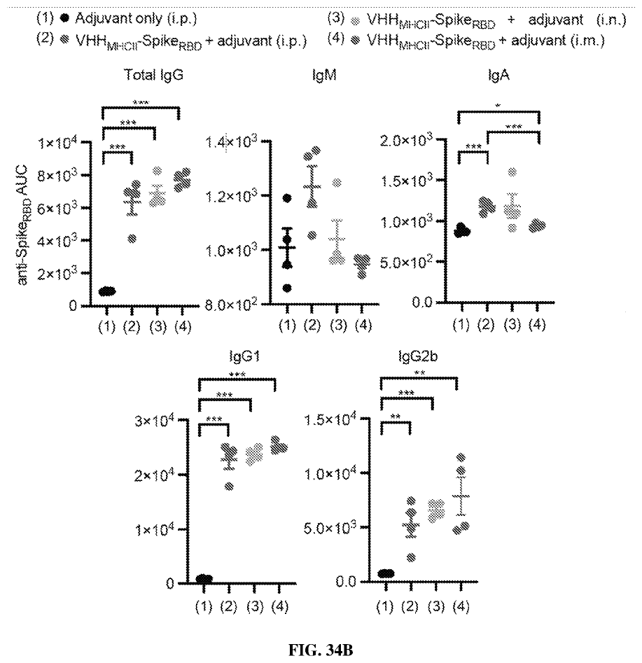

With the scale of the SARS-CoV-2 (COVID-19) pandemic, immunization with three or more doses is likely to be impractical, yet a strong CD8+ T cell response may be possible by immunization with a single dose. Therefore, an experiment was conducted in which animals received two successive doses of VHH_{MHCII}-Spike_{RBD} (FIG. 33A). Serum immunoglobulins were tracked post-immunization at days 7, 14 and 21 (FIG. 33A). Total IgG in the VHH_{MHCII}-Spike_{R}BD cohort reached peak levels on day 7 after the second dose and these levels persisted through day 21. Animals which received a single dose of the VHH_{MHCII}-Spike_{RBD} preparation and adjuvant demonstrated lower efficacy than the double dose of VHH_{MHCII}-Spike_{RBD} at all timepoints (FIG. 33A). Isotype switching was verified as well (FIG. 33B). Sera from immunized animals was then tested for recognition of Spike_{RBD} that carry the K417T, E484K, N501Y mutations. Sera obtained at day 14 efficiently recognized this variant Spike_{RBD} (FIG. 33C). Therefore, sera from these animals was tested for neutralization of pseudo-typed VSV carrying a diverse set of Spike variants. Sera from mice immunized with 2 doses of VHH_{MHCII}-Spike_{RBD} effectively neutralized all variants tested (FIG. 33D).

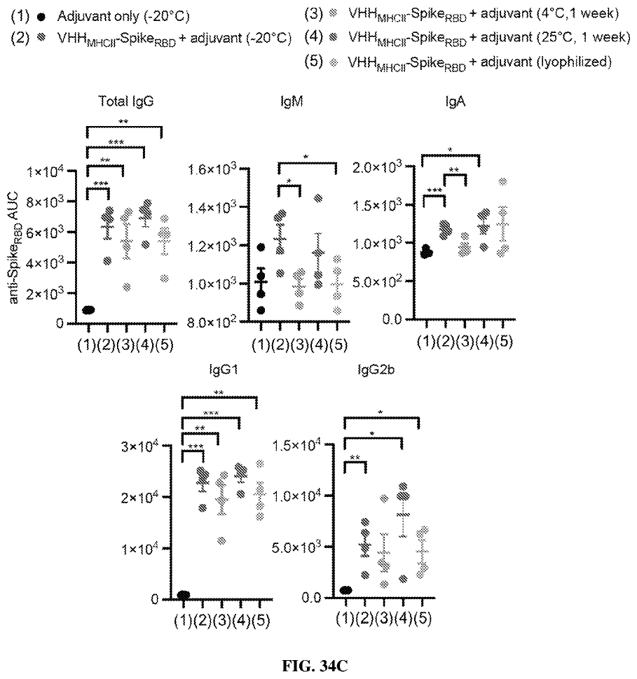

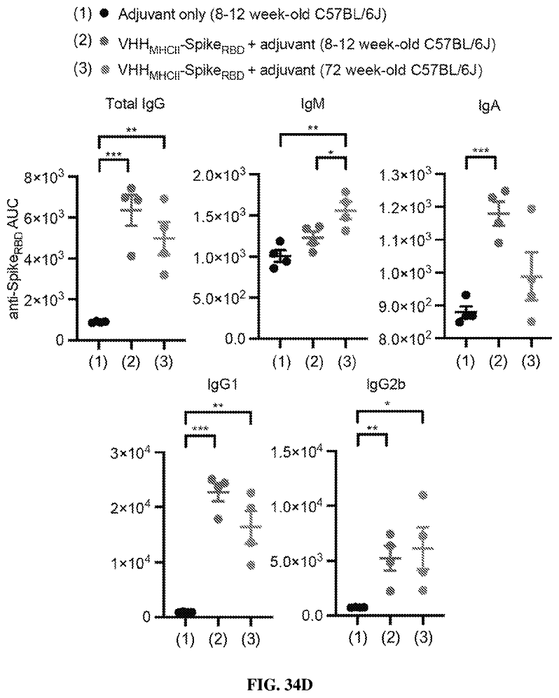

The experiments described in FIGS. 31-33 all rely on intraperitoneal delivery of the vaccine preparation. For use in humans, intramuscular delivery is preferred. A needle-free approach, e.g., intranasal delivery, would be a highly desirable alternative to injection. It was therefore investigated whether different delivery routes would lead to different levels of antibody production when administered in 2 doses with a 2-week interval (FIG. 34A). The VHH_{MHCII}-Spike_{RBD} preparation was delivered either intraperitoneally (i.p), intramuscularly (i.m), or intranasally (i.n). Whereas i.p. and i.m delivery elicited an IgA response, intranasal delivery failed to do so (FIG. 34B). However, serum IgG production appeared to be independent of the route of vaccine delivery. All three routes of delivery yielded similar levels of total anti-Spike_{RBD} IgG (FIG. 34B).

It was then explored whether the VHH_{MHCII}-Spike_{RBD} vaccine preparation could survive room temperature storage and lyophilization, yielding a final ‘dry’ product at room temperature, without loss of potency. All methods of storage that were tested produced equivalent levels of total IgG, in addition to other Ig isotypes previously observed (FIG. 34C).

Another key consideration is whether a VHH_{MHCII}-Spike_{RBD} vaccine would perform well for all age classes, in particular for aged individuals. The VHH_{MHCII}-Spike_{RBD} vaccine was therefore tested in aged mice (72 weeks old, equivalent to human age 56-69 years old), wherein it demonstrated a robust total antibody response against the Spike_{RBD} (FIG. 34D).

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A composition comprising:
(i) a conjugate comprising a single domain antibody (VHH) conjugated to an antigen and an anti-inflammatory agent, wherein the VHH binds to a surface protein on an antigen presenting cell (APC); or
(ii) a first conjugate comprising a VHH conjugated to an antigen and a second conjugate comprising a second VHH conjugated to an anti-inflammatory agent, wherein the first VHH and the second VHH bind to one or more surface proteins on an antigen presenting cell (APC).

2. The composition of claim 1, wherein the surface protein on the APC is selected from the group consisting of MHCII, CD11c, DEC205, DC-SIGN, CLEC9a, CD103, CX3CR1, CD1a, and F4/80.

3. The composition of claim 2, wherein the composition comprises a conjugate comprising a VHH to conjugated to an antigen and an anti-inflammatory agent, wherein the VHH binds to MHCII.

4. The composition of claim 2, wherein the composition comprises a first conjugate comprising a first VHH conjugated to an antigen and a second conjugate comprising a second VHH conjugated to an anti-inflammatory agent, wherein the first VHH and the second VHH both bind to MHCII.

5. The composition of claim 3 or claim 4, wherein the VHH comprises the amino acid sequences of SEQ ID NO: 1.

6. The composition of any one of claims 1-5, wherein the VHH further comprises a sortase recognition sequence at the N-terminus or C-terminus.

7. The composition of claim 6, wherein the sortase recognition sequence comprises LPETG (SEQ ID NO: 29), optionally wherein the sortase recognition sequence comprises LPETGG (SEQ ID NO: 43).

8. The composition of claim 6 or claim 7, wherein an anti-inflammatory agent or an antigen is conjugated to the VHH via the sortase recognition sequence.

9. The composition of any one of claims 1-8, wherein the anti-inflammatory agent further comprises a hydrolysable or non-hydrolysable linker.

10. The composition of claim 2, wherein the composition comprises a conjugate comprising a single domain antibody (VHH) conjugated to an antigen and an anti-inflammatory agent, wherein the VHH binds to CD11c.

11. The composition of claim 2, wherein the composition comprises a first conjugate comprising a first VHH conjugated to an antigen and a second conjugate comprising a second VHH conjugated to an anti-inflammatory agent, wherein the first VHH and the second VHH both bind to CD11c.

12. The composition of claim 10 or claim 11, wherein the VHH comprises the amino acid sequences of SEQ ID NO: 2.

13. The composition of any one of claims 10-12, wherein the VHH further comprises a sortase recognition sequence at the N-terminus or C-terminus.

14. The composition of claim 13, wherein the sortase recognition sequence comprises LPETG (SEQ ID NO: 29), optionally wherein the sortase recognition sequence comprises LPETGG (SEQ ID NO: 43).

15. The composition of claim 13 or claim 14, wherein an anti-inflammatory agent or an antigen is conjugated to the VHH via the sortase recognition sequence.

16. The composition of any one of claims 10-15 wherein the anti-inflammatory agent further comprises a hydrolysable or non-hydrolysable linker.

17. The composition of claim 2, wherein the composition comprises a first conjugate comprising a first VHH conjugated to an antigen and a second conjugate comprising a second VHH conjugated to an anti-inflammatory agent, wherein the first VHH and the second VHH bind to different surface proteins on the APC.

18. The composition of claim 17, wherein the first VHH binds to MHCII and the second VHH binds to CD11c.

19. The composition of claim 17, wherein the first VHH binds to DEC205 and the second VHH binds to MHCII.

20. The composition of any one of claims 1-19, wherein the anti-inflammatory agent is a steroidal anti-inflammatory agent selected from the group consisting of: dexamethasone, prednisone, prednisolone, triamcinolone, methylprednisolone, and bethamethasone.

21. The composition of any one of claims 1-19, wherein the anti-inflammatory agent is a nonsteroidal anti-inflammatory agent selected from the group consisting of: aspirin, celecoxib, diclofenac, ibuprofen, ketoprofen, naproxen, oxaprozin, piroxicam, cyclosporin A, and calcitriol.

22. The composition of any one of claims 1-19, wherein the anti-inflammatory agent is an anti-inflammatory cytokine selected from the group consisting of IL-10, IL-35, IL-4, IL-11, IL-13, and TGFβ.

23. The composition of any one of claims 1-22, wherein the antigen comprises a polypeptide, a polysaccharide, a carbohydrate, a lipid, a nucleic acid, or combination thereof.

24. The composition of any one of claims 1-23, wherein the antigen is a self-antigen.

25. The composition of claim 24, wherein the self-antigen is selected from myelin oligodendrocyte glycoprotein, myelin proteolipid protein, citrullinated fibrinogen, insulin, chromogranin A, glutamic acid decarboxylase 65-kilodalton isoform (GAD65), desmoglein 1 (DSG1), desmoglein 3 (DSG3), acetylcholine receptor (AChR), muscle-specific tyrosine kinase (MuSK), ribonucleoproteins.

26. The composition of claim 23, wherein the antigen comprises a protein used in a protein replacement therapy or a gene therapy.

27. The composition of claim 26, wherein the antigen is selected from Factor IX, Factor VIII, insulin, and AAV-derived proteins.

28. A method comprising administering to a subject in need thereof the composition of any one of claims 1-27.

29. A method of inducing immune tolerance to an antigen, the method comprising administering to a subject in need thereof the composition of any one of claims 1-27.

30. A method of treating an autoimmune disease, the method comprising administering to a subject in need thereof the composition of any one of claims 1-25.

31. The method of claim 30, wherein the autoimmune disease is selected from the group consisting of autoimmune encephalomyelitis, multiple sclerosis, type I diabetes, Pemphigus vulgaris, myasthenia gravis, lupus, celiac diseases, and inflammatory bowel disease (IBD).

32. The method of any one of claims 28-31, wherein the administration is intravenous.

33. The method of any one of claims 28-32, wherein the subject is human.

34. A composition comprising:
(i) a conjugate comprising a single domain antibody (VHH) conjugated to an antigen and a pro-inflammatory agent, wherein the VHH binds to a surface protein on an antigen presenting cell (APC); or
(ii) a first conjugate comprising a VHH conjugated to an antigen and a second conjugate comprising a second VHH conjugated to a pro-inflammatory agent, wherein the first VHH and the second VHH bind to one or more surface proteins on an antigen presenting cell (APC).

35. The composition of claim 34, wherein the surface protein on the APC is selected from the group consisting of MHCII, CD11c, DEC205, DC-SIGN, CLEC9a, CD103, CX3CR1, CD1a, and F4/80.

36. The composition of claim 35, wherein the composition comprises a conjugate comprising a single domain antibody (VHH) conjugated to an antigen and a pro-inflammatory agent, wherein the VHH binds to MHCII.

37. The composition of claim 35, wherein the composition comprises a first conjugate comprising a first VHH conjugated to an antigen and a second conjugate comprising a second VHH conjugated to a pro-inflammatory agent, wherein the first VHH and the second VHH both bind to MHCII.

38. The composition of claim 36 or claim 37, wherein the VHH comprises the amino acid sequences of SEQ ID NO: 1.

39. The composition of any one of claims 34-38, wherein the VHH further comprises a sortase recognition sequence at the N-terminus or C-terminus.

40. The composition of claim 39, wherein the sortase recognition sequence comprises LPETG (SEQ ID NO: 29), optionally wherein the sortase recognition sequence comprises LPETGG (SEQ ID NO: 43).

41. The composition of claim 39 or claim 40, wherein a pro-inflammatory agent or an antigen is conjugated to the VHH via the sortase recognition sequence.

42. The composition of any one of claims 34-41, wherein the pro-inflammatory agent further comprises a hydrolysable or non-hydrolysable linker.

43. The composition of claim 35, wherein the composition comprises a conjugate comprising a single domain antibody (VHH) conjugated to an antigen and a pro-inflammatory agent, wherein the VHH binds to CD11c.

44. The composition of claim 35, wherein the composition comprises a first conjugate comprising a first VHH conjugated to an antigen and a second conjugate comprising a second VHH conjugated to a pro-inflammatory agent, wherein the first VHH and the second VHH both bind to CD11c.

45. The composition of claim 43 or claim 44, wherein the VHH comprises the amino acid sequences of SEQ ID NO: 2.

46. The composition of any one of claims 43-45, wherein the VHH further comprises a sortase recognition sequence at the N-terminus or C-terminus.

47. The composition of claim 46, wherein the sortase recognition sequence comprises LPETG (SEQ ID NO: 29), optionally wherein the sortase recognition sequence comprises LPETGG (SEQ ID NO: 43).

48. The composition of claim 46 or claim 47, wherein a pro-inflammatory agent or an antigen is conjugated to the VHH via the sortase recognition sequence.

49. The composition of any one of claims 43-48, wherein the pro-inflammatory agent further comprises a hydrolysable or non-hydrolysable linker.

50. The composition of claim 35, wherein the composition comprises a first conjugate comprising a first VHH conjugated to an antigen and a second conjugate comprising a second VHH conjugated to a pro-inflammatory agent, wherein the first VHH and the second VHH bind to different surface proteins on the APC.

51. The composition of claim 50, wherein the first VHH binds to MHCII and the second VHH binds to CD11c.

52. The composition of claim 50, wherein the first VHH binds to DEC205 and the second VHH binds to MHCII.

53. The composition of any one of claims 34-52, wherein the pro-inflammatory agent is selected from the group consisting of: TLR9 agonist, LPS, HMGB1 proteins, IL2, IL12, and CD40L.

54. The composition of any one of claims 34-53, wherein the antigen comprises a polypeptide, a polysaccharide, a carbohydrate, a lipid, a nucleic acid, or combination thereof.

55. The composition of any one of claims 34-54, wherein the antigen is from a microbial pathogen.

56. The composition of claim 55, wherein the microbial pathogen is a mycobacterium, bacterium, fungus, virus, parasite, or prion.

57. The composition of any one of claims 34-56, wherein the antigen comprises a SARS-CoV-2 spike protein.

58. The composition of any one of claims 34-54, wherein the antigen is a tumor antigen.

59. The composition of any one of claims 34-58, wherein the composition is a vaccine composition.

60. A method comprising administering to a subject in need thereof the composition of any one of claims 34-59.

61. A method of inducing immune response to an antigen, the method comprising administering to a subject in need thereof the composition of any one of claims 34-59.

62. A method of treating infection caused by a pathogen, the method comprising administering to a subject in need thereof the composition of any one of claims 34-59, wherein the antigen is from the microbial pathogen.

63. The method of claim 62, wherein the method is therapeutic or prophylactic.

64. A method of treating cancer, the method comprising administering to a subject in need thereof the composition of any one of claims 34-59, wherein the antigen is a tumor antigen.

65. The method of any one of claims 60-64, wherein the administration is intravenous.

66. The method of any one of claims 60-65, wherein the subject is human.


SEQUENCE LISTING (61 sequences)

SEQ ID NO: 1 - protein, 129 aa, Artificial Sequence
  Synthetic Polypeptide
QVQLQESGGGLVQAGDSLRLSCAASGRTFSRGVMGWFRRAPGKEREFVAIFSGSSWSGRSTYYSDSVKGRFTISRDNAKNTVYLQMNGLKPEDTAVYYCAAGYPEAYSAYGRESTYDYWGQGTQVTVSS

SEQ ID NO: 2 - protein, 121 aa, Artificial Sequence
  Synthetic Polypeptide
EVQLVESGGGLVQAGGSLRLSCRVSGLPFSNLILGWLRQAPGKEREFVARISRSDSTYYEQFAEGRFTISRDNAKNTAYLHLNDLKPEDTAVYYCAAANPIFRSYDDYDYWGQGTQVTVSS

SEQ ID NO: 3 - protein, 206 aa, Artificial Sequence
  Synthetic Polypeptide
MKKWTNRLMTIAGVVLILVAAYLFAKPHIDNYLHDKDKDEKIEQYDKNVKEQASKDNKQQAKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVEVLDEQKGKDKQLTLITCDDYNEKTGVWEKRKIFVATEVK

SEQ ID NO: 4 - protein, 29 aa, Artificial Sequence
  Synthetic Polypeptide
GGGCKKGSMEVGWYRSPFSRVVHLYRNGK

SEQ ID NO: 5 - protein, 16 aa, Artificial Sequence
  Synthetic Polypeptide
GGGCKKFRSIINFEKL

SEQ ID NO: 6 - protein, 23 aa, Artificial Sequence
  Synthetic Polypeptide
GGGCKKISQAVHAAHAEINEAGR

SEQ ID NO: 7 - protein, 23 aa, Artificial Sequence
  Synthetic Polypeptide
GGGCKKFDKLPGFGDSIEAQGGK

SEQ ID NO: 8 - protein, 16 aa, Artificial Sequence
  Synthetic Polypeptide
GGGCKKYVRPLWVRME

SEQ ID NO: 9 - protein, 17 aa, Artificial Sequence
  Synthetic Polypeptide
GGGCKKFRKNYIFEEKL

SEQ ID NO: 10 - protein, 1255 aa, Artificial Sequence
  Synthetic Polypeptide
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - protein, 222 aa, Artificial Sequence
  Synthetic Polypeptide
RVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNF

SEQ ID NO: 12 - protein, 76 aa, Artificial Sequence
  Synthetic Polypeptide
MYSFVSEETGTLIVNSVLLFLAFVVFLLVTLAILTALRLCAYCCNIVNVSLVKPTVYVYSRVKNLNSSEGVPDLLV

SEQ ID NO: 13 - protein, 82 aa, Artificial Sequence
  Synthetic Polypeptide
MLPFVQERIGLFIVNFFIFTVVCAITLLVCMAFLTATRLCVQCMTGFNTLLVQPALYLYNTGRSVYVKFQDSKPPLPPDEWV

SEQ ID NO: 14 - protein, 221 aa, Artificial Sequence
  Synthetic Polypeptide
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 15 - protein, 422 aa, Artificial Sequence
  Synthetic Polypeptide
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 16 - protein, 1273 aa, Artificial Sequence
  Synthetic Polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 223 aa, Artificial Sequence
  Synthetic Polypeptide
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 18 - protein, 75 aa, Artificial Sequence
  Synthetic Polypeptide
MYSFVSEETGTLIVNSVLLFLAFVVFLLVTLAILTALRLCAYCCNIVNVSLVKPSFYVYSRVKNLNSSRVPDLLV

SEQ ID NO: 19 - protein, 222 aa, Artificial Sequence
  Synthetic Polypeptide
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 20 - protein, 419 aa, Artificial Sequence
  Synthetic Polypeptide
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 21 - protein, 1353 aa, Artificial Sequence
  Synthetic Polypeptide
MIHSVFLLMFLLTPTESYVDVGPDSVKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSTVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFRKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 22 - protein, 122 aa, Artificial Sequence
  Synthetic Polypeptide
ECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKC

SEQ ID NO: 23 - protein, 219 aa, Artificial Sequence
  Synthetic Polypeptide
MSNMTQLTEAQIIAIIKDWNFAWSLIFLLITIVLQYGYPSRSMTVYVFKMFVLWLLWPSSMALSIFSAVYPIDLASQIISGIVAAVSAMMWISYFVQSIRLFMRTGSWWSFNPETNCLLNVPFGGTTVVRPLVEDSTSVTAVVTNGHLKMAGMHFGACDYDRLPNEVTVAKPNVLIALKMVKRQSYGTNSGVAIYHRYKAGNYRSPPITADIELALLRA

SEQ ID NO: 24 - protein, 413 aa, Artificial Sequence
  Synthetic Polypeptide
MASPAAPRAVSFADNNDITNTNLSRGRGRNPKPRAAPNNTVSWYTGLTQHGKVPLTFPPGQGVPLNANSTPAQNAGYWRRQDRKINTGNGIKQLAPRWYFYYTGTGPEAALPFRAVKDGIVWVHEDGATDAPSTFGTRNPNNDSAIVTQFAPGTKLPKNFHIEGTGGNSQSSSRASSLSRNSSRSSSQGSRSGNSTRGTSPGPSGIGAVGGDLLYLDLLNRLQALESGKVKQSQPKVITKKDAAAAKNKMRHKRTSTKSFNMVQAFGLRGPGDLQGNFGDLQLNKLGTEDPRWPQIAELAPTASAFMGMSQFKLTHQNNDDHGNPVYFLRYSGAIKLDPKNPNYNKWLELLEQNIDAYKTFPKKEKKQKAPKEESTDQMSEPPKEQRVQGSITQRTRTRPSVQPGPMIDVNTD

SEQ ID NO: 25 - protein, 5 aa, Artificial Sequence
  Synthetic Polypeptide
  misc_feature (3)..(3): Xaa can be any naturally occurring amino acid
LPXTG

SEQ ID NO: 26 - protein, 5 aa, Artificial Sequence
  Synthetic Polypeptide
LPKTG

SEQ ID NO: 27 - protein, 5 aa, Artificial Sequence
  Synthetic Polypeptide
LPATG

SEQ ID NO: 28 - protein, 5 aa, Artificial Sequence
  Synthetic Polypeptide
LPNTG

SEQ ID NO: 29 - protein, 5 aa, Artificial Sequence
  Synthetic Polypeptide
LPETG

SEQ ID NO: 30 - protein, 5 aa, Artificial Sequence
  Synthetic Polypeptide
  misc_feature (3)..(3): Xaa can be any naturally occurring amino acid
LPXAG

SEQ ID NO: 31 - protein, 5 aa, Artificial Sequence
  Synthetic Polypeptide
LPNAG

SEQ ID NO: 32 - protein, 5 aa, Artificial Sequence
  Synthetic Polypeptide
  misc_feature (3)..(3): Xaa can be any naturally occurring amino acid
LPXTA

SEQ ID NO: 33 - protein, 5 aa, Artificial Sequence
  Synthetic Polypeptide
LPNTA

SEQ ID NO: 34 - protein, 5 aa, Artificial Sequence
  Synthetic Polypeptide
  misc_feature (3)..(3): Xaa can be any naturally occurring amino acid
LGXTG

SEQ ID NO: 35 - protein, 5 aa, Artificial Sequence
  Synthetic Polypeptide
LGATG

SEQ ID NO: 36 - protein, 5 aa, Artificial Sequence
  Synthetic Polypeptide
  misc_feature (3)..(3): Xaa can be any naturally occurring amino acid
IPXTG

SEQ ID NO: 37 - protein, 5 aa, Artificial Sequence
  Synthetic Polypeptide
IPNTG

SEQ ID NO: 38 - protein, 5 aa, Artificial Sequence
  Synthetic Polypeptide
IPETG

SEQ ID NO: 39 - protein, 5 aa, Artificial Sequence
  Synthetic Polypeptide
  misc_feature (3)..(3): Xaa can be any naturally occurring amino acid
  misc_feature (5)..(5): Xaa can be any naturally occurring amino acid
NPXTX

SEQ ID NO: 40 - protein, 5 aa, Artificial Sequence
  Synthetic Polypeptide
NPQTN

SEQ ID NO: 41 - protein, 5 aa, Artificial Sequence
  Synthetic Polypeptide
NPKTG

SEQ ID NO: 42 - protein, 18 aa, Artificial Sequence
  Synthetic Polypeptide
YIYFNTWTTCQSIAFPSK

SEQ ID NO: 43 - protein, 6 aa, Artificial Sequence
  Synthetic Polypeptide
LPETGG

SEQ ID NO: 44 - protein, 5 aa, Artificial Sequence
  Synthetic Polypeptide
NSKTA

SEQ ID NO: 45 - protein, 5 aa, Artificial Sequence
  Synthetic Polypeptide
NPQTG

SEQ ID NO: 46 - protein, 5 aa, Artificial Sequence
  Synthetic Polypeptide
NAKTN

SEQ ID NO: 47 - protein, 5 aa, Artificial Sequence
  Synthetic Polypeptide
NPQSS

SEQ ID NO: 48 - protein, 5 aa, Artificial Sequence
  Synthetic Polypeptide
  misc_feature (3)..(3): Xaa can be any naturally occurring amino acid
LAXTG

SEQ ID NO: 49 - protein, 21 aa, Artificial Sequence
  Synthetic Polypeptide
MEVGWYRSPFSRVVHLYRNGK

SEQ ID NO: 50 - protein, 15 aa, Artificial Sequence
  Synthetic Polypeptide
QDFTNCITINKLKNS

SEQ ID NO: 51 - protein, 21 aa, Artificial Sequence
  Synthetic Polypeptide
GIVEQCCTSICSLYQLENYCN

SEQ ID NO: 52 - protein, 30 aa, Artificial Sequence
  Synthetic Polypeptide
FVNQHLCGSHLVEALYLVCGERGFFYTPKT

SEQ ID NO: 53 - protein, 12 aa, Homo sapiens
TCFFRDHSYQEE

SEQ ID NO: 54 - protein, 6 aa, Artificial Sequence
  Synthetic Polypeptide
  misc_feature (3)..(3): Xaa can be any naturally occurring amino acid
LPXTGG

SEQ ID NO: 55 - protein, 7 aa, Artificial Sequence
  Synthetic Polypeptide
  misc_feature (3)..(3): Xaa can be any naturally occurring amino acid
LPXTGGG

SEQ ID NO: 56 - protein, 15 aa, Artificial Sequence
  Synthetic Polypeptide
VEGFNCYFPLQSYGF

SEQ ID NO: 57 - protein, 15 aa, Artificial Sequence
  Synthetic Polypeptide
VGYQPYRVVVLSFEL

SEQ ID NO: 58 - protein, 15 aa, Artificial Sequence
  Synthetic Polypeptide
PYRVVVLSFELLHAP

SEQ ID NO: 59 - protein, 15 aa, Artificial Sequence
  Synthetic Polypeptide
VVLSFELLHAPATVC

SEQ ID NO: 60 - protein, 15 aa, Artificial Sequence
  Synthetic Polypeptide
FELLHAPATVCGPKK

SEQ ID NO: 61 - protein, 4 aa, Artificial Sequence
  Synthetic Polypeptide
GGGC